SEQUENCE LISTING (59 sequences)

SEQ ID NO: 1 - DNA, 2983 bp, Homo sapiens
gtggctcttctggcccgggctactatatagagacgtttccgcctcctgcttgaaactaacccctctttttctccaaaggagtgcttgtggagatcggatcttttctccagcaattgggggaaagaaggctttttctctgaattagcttagtgtaaccagcggcgtatattttttaggcgccttttcgaaaacctagtagttaatattcatttgtttaaatcttattttatttttaagctcaaactgcttaagaataccttaattccttaaagtgaaataattttttgcaaaggggtttcctcgatttggagctttttttttcttccaccgtcatttctaactcttaaaaccaactcagttccatcatggtgatgttcaagaagatcaagtcttttgaggtggtctttaacgaccctgaaaaggtgtacggcagtggcgagaaggtggctggccgggtgatagtggaggtgtgtgaagttactcgtgtcaaagccgttaggatcctggcttgcggagtggctaaagtgctttggatgcagggatcccagcagtgcaaacagacttcggagtacctgcgctatgaagacacgcttcttctggaagaccagccaacaggtgagaatgagatggtgatcatgagacctggaaacaaatatgagtacaagttcggctttgagcttcctcaggggcctctgggaacatccttcaaaggaaaatatgggtgtgtagactactgggtgaaggcttttcttgaccgcccgagccagccaactcaagagacaaagaaaaactttgaagtagtggatctggtggatgtcaatacccctgatttaatggcacctgtgtctgctaaaaaagaaaagaaagtttcctgcatgttcattcctgatgggcgggtgtctgtctctgctcgaattgacagaaaaggattctgtgaaggtgatgagatttccatccatgctgactttgagaatacatgttcccgaattgtggtccccaaagctgccattgtggcccgccacacttaccttgccaatggccagaccaaggtgctgactcagaagttgtcatcagtcagaggcaatcatattatctcagggacatgcgcatcatggcgtggcaagagccttcgggttcagaagatcaggccttctatcctgggctgcaacatccttcgagttgaatattccttactgatctatgttagcgttcctggatccaagaaggtcatccttgacctgcccctggtaattggcagcagatcaggtctaagcagcagaacatccagcatggccagccgaaccagctctgagatgagttgggtagatctgaacatccctgataccccagaagctcctccctgctatatggatgtcattcctgaagatcaccgattggagagcccaaccactcctctgctagatgacatggatggctctcaagacagccctatctttatgtatgcccctgagttcaagttcatgccaccaccgacttatactgaggtggatccctgcatcctcaacaacaatgtgcagtgagcatgtggaagaaaagaagcagctttacctacttgtttctttttgtctctcttcctggacactcactttttcagagactcaacagtctctgcaatggagtgtgggtccaccttagcctctgacttcctaatgtaggaggtggtcagcaggcaatctcctgggccttaaaggatgcggactcatcctcagccagcgcccatgttgtgatacaggggtgtttgttggatgggtttaaaaataactagaaaaactcaggcccatccattttctcagatctccttgaaaattgaggccttttcgatagtttcgggtcaggtaaaaatggcctcctggcgtaagcttttcaaggttttttggaggctttttgtaaattgtgataggaactttggaccttgaacttacgtatcatgtggagaagagccaatttaacaaactaggaagatgaaaagggaaattgtggccaaaactttgggaaaaggaggttcttaaaatcagtgtttcccctttgtgcacttgtagaaaaaaaagaaaaaccttctagagctgatttgatggacaatggagagagctttccctgtgattataaaaaaggaagctagctgctctacggtcatctttgcttagagtatactttaacctggcttttaaagcagtagtaactgccccaccaaaggtcttaaaagccatttttggagcctattgcactgtgttctcctactgcaaatattttcatatgggaggatggttttctcttcatgtaagtccttggaattgattctaaggtgatgttcttagcactttaattcctgtcaaattttttgttctccccttctgccatcttaaatgtaagctgaaactggtctactgtgtctctagggttaagccaaaagacaaaaaaaattttactacttttgagattgccccaatgtacagaattatataattctaacgcttaaatcatgtgaaagggttgctgctgtcagccttgcccactgtgacttcaaacccaaggaggaactcttgatcaagatgcccaaccctgtgatcagaacctccaaatactgccatgagaaactagagggcaggtcttcataaaagccctttgaacccccttcctgccctgtgttaggagatagggatattggcccctcactgcagctgccagcacttggtcagtcactctcagccatagcactttgttcactgtcctgtgtcagagcactgagctccacccttttctgagagttattacagccagaaagtgtgggctgaagatggttggtttcatgtttttgtattatgtatctttttgtatggtaaagactatattttgtacttaaccagatatatttttaccccagatggggatattctttgtaaaaaatgaaaataaagtttttttaatggaaaaaaaaatgtctgtgaaaaaaaaaaaaaaaaaa

SEQ ID NO: 2 - DNA, 2614 bp, Homo sapiens
gagatgttagtagagaactgtcctgggtgaatctttcagtattgcagggcttggcaacttgctgcccgacaaaatacatcagaatttctctttaagaacaatatgggatggattaaaaaatatatatatgggatgaaattgggggtacttcaataccttgcatgccacccaagcattccttatcacacagatgcattttaagtgtaacagcaagcctaatggctactcgattttctttcccttcaggtgagaatgagatggtgatcatgagacctggaaacaaatatgagtacaagttcggctttgagcttcctcaggggcctctgggaacatccttcaaaggaaaatatgggtgtgtagactactgggtgaaggcttttcttgaccgcccgagccagccaactcaagagacaaagaaaaactttgaagtagtggatctggtggatgtcaatacccctgatttaatggcacctgtgtctgctaaaaaagaaaagaaagtttcctgcatgttcattcctgatgggcgggtgtctgtctctgctcgaattgacagaaaaggattctgtgaaggtgatgagatttccatccatgctgactttgagaatacatgttcccgaattgtggtccccaaagctgccattgtggcccgccacacttaccttgccaatggccagaccaaggtgctgactcagaagttgtcatcagtcagaggcaatcatattatctcagggacatgcgcatcatggcgtggcaagagccttcgggttcagaagatcaggccttctatcctgggctgcaacatccttcgagttgaatattccttactgatctatgttagcgttcctggatccaagaaggtcatccttgacctgcccctggtaattggcagcagatcaggtctaagcagcagaacatccagcatggccagccgaaccagctctgagatgagttgggtagatctgaacatccctgataccccagaagctcctccctgctatatggatgtcattcctgaagatcaccgattggagagcccaaccactcctctgctagatgacatggatggctctcaagacagccctatctttatgtatgcccctgagttcaagttcatgccaccaccgacttatactgaggtggatccctgcatcctcaacaacaatgtgcagtgagcatgtggaagaaaagaagcagctttacctacttgtttctttttgtctctcttcctggacactcactttttcagagactcaacagtctctgcaatggagtgtgggtccaccttagcctctgacttcctaatgtaggaggtggtcagcaggcaatctcctgggccttaaaggatgcggactcatcctcagccagcgcccatgttgtgatacaggggtgtttgttggatgggtttaaaaataactagaaaaactcaggcccatccattttctcagatctccttgaaaattgaggccttttcgatagtttcgggtcaggtaaaaatggcctcctggcgtaagcttttcaaggttttttggaggctttttgtaaattgtgataggaactttggaccttgaacttacgtatcatgtggagaagagccaatttaacaaactaggaagatgaaaagggaaattgtggccaaaactttgggaaaaggaggttcttaaaatcagtgtttcccctttgtgcacttgtagaaaaaaaagaaaaaccttctagagctgatttgatggacaatggagagagctttccctgtgattataaaaaaggaagctagctgctctacggtcatctttgcttagagtatactttaacctggcttttaaagcagtagtaactgccccaccaaaggtcttaaaagccatttttggagcctattgcactgtgttctcctactgcaaatattttcatatgggaggatggttttctcttcatgtaagtccttggaattgattctaaggtgatgttcttagcactttaattcctgtcaaattttttgttctccccttctgccatcttaaatgtaagctgaaactggtctactgtgtctctagggttaagccaaaagacaaaaaaaattttactacttttgagattgccccaatgtacagaattatataattctaacgcttaaatcatgtgaaagggttgctgctgtcagccttgcccactgtgacttcaaacccaaggaggaactcttgatcaagatgcccaaccctgtgatcagaacctccaaatactgccatgagaaactagagggcaggtcttcataaaagccctttgaacccccttcctgccctgtgttaggagatagggatattggcccctcactgcagctgccagcacttggtcagtcactctcagccatagcactttgttcactgtcctgtgtcagagcactgagctccacccttttctgagagttattacagccagaaagtgtgggctgaagatggttggtttcatgtttttgtattatgtatctttttgtatggtaaagactatattttgtacttaaccagatatatttttaccccagatggggatattctttgtaaaaaatgaaaataaagtttttttaatggaaaaaaaaatgtctgtgaaaaaaaaaaaaaaaaaa

SEQ ID NO: 3 - DNA, 2801 bp, Mus musculus
gacactctcctcctctggtctcggggtttccagagtttctccagttgcggaagacagctgttatttttctcctgaaagcttttggcacagccggcaggctgaaacttccaggcaccttttggaaaagttgttagggtttgtttgaagctttctttacattttcgtttgggttttcaagccctgactttacggaggcgagctcttcgtttgctttgaagggttcttaaagatttttttcctctccggctttcgtttttcttgaacccactcggctcaatcatggtgatgttcaagaagatcaagtcttttgaggtggtcttcaacgaccccgagaaggtgtacggcagcggggagaaggtggccggacgggtaatagtggaagtgtgtgaagttacccgagtcaaagccgtcaggatcctggcttgcggcgtggccaaggtcctgtggatgcaagggtctcagcagtgcaaacagactttggactacttgcgctatgaagacacacttctcctagaagagcagcctacagcaggtgagaacgagatggtgatcatgaggcctggaaacaaatatgagtacaagttcggcttcgagcttcctcaagggcccctgggaacatcctttaaaggaaaatatggttgcgtagactactgggtgaaggcttttctcgatcgccccagccagccaactcaagaggcaaagaaaaacttcgaagtgatggatctagtggatgtcaatacccctgacctaatggcaccagtgtctgccaaaaaggagaagaaagtttcctgcatgttcattcctgatggacgtgtgtcagtctctgctcgaattgacagaaaaggattctgtgaaggtgatgacatctccatccatgctgactttgagaacacgtgttcccgaatcgtggtccccaaagcggctattgtggcccgacacacttaccttgccaatggccagaccaaagtgttcactcagaagctgtcctcagtcagaggcaatcacattatctcagggacttgcgcatcgtggcgtggcaagagcctcagagtgcagaagatcagaccatccatcctgggctgcaacatcctcaaagtcgaatactccttgctgatctacgtcagtgtccctggctccaagaaagtcatccttgatctgcccctagtgattggcagcaggtctggtctgagcagccggacatccagcatggccagccggacgagctctgagatgagctggatagacctaaacatcccagataccccagaagctcctccttgctatatggacatcattcctgaagatcacagactagagagccccaccacccctctgctggacgatgtggacgactctcaagacagccctatctttatgtacgcccctgagttccagttcatgcccccacccacttacactgaggtggatccgtgcgtccttaacaacaacaacaacaacaacaacgtgcagtgagcctgcaggaaatgaagcatctgtattagcgcatttctttctgcctctctgcttgaactccagtgtttcagagactcagtctctacagcggggaacgggtacaccccagccgctgactcctcaagatgggtggcaatcagtaggcgggtctccggcttcaagtggtgcagaccagtgcccgcactgtggcataggagtgtttgctgggtggatgtcagaacactcttagaaaaattgagacctgaccactttctcggatgttggaaatgaagaacttgtttgtgttgactgagtcagggcactgctgaccttctggcgttgtctttccaaggtttttgttttaaagggacttttaaattgtctaaaatatcagtagaccatcatctgtgccatgggggacagagccaatttcaagtcatggccaaaattttgtaagaggagtgtttttgtgtgttttttaaagtcagtgttccttttttatatctttacaaagaaaagaccttccacggctggtgagcacgcagcctgtgaaattcggggcagctgctccaagttgacttcaccctgggagcagtagtagctgtgcccactgacggccataaaagccattttacagccagttgcactgtgttctcttgtaagcataatcagatgggagaatctgttatttccctgtaaccccttggaattgattctaaggtgatgttcttagcactttagcttgtcaattttgttttagtctccgttatagatgtaagctccaccagtctcttaaggattaagcccagtgacttggagggtgggggttagggtctctatccctgaacattgtagacccaggctggcctgagagatccacctgcctctgcctcctgagtgctgcgatcaaaggcccagcttggttattgcttttgaggctttctcccaacgcacagacttgtgtaattctaacactaatcctgtgaagggttgtggttgacagctggagcctgggtgacattctacattgagatgccccagcactgatcggggcacagaagcccccagaccccatttcctgtccagtgttgggagaaagtgctgctttcactgtggcctcagccctggctcggaagctcactaagccttagcactttgtcctgtgtcagctccacctgagaactgtgcagccagaatgtctgcgagctgatggaggtttcggttttgttgtttttgtattttgtgtatctttttgtatgattaaaaactatattttctacttatccaaatatattttcaccccaaagtggggttatcctttgtaaaaaaaaataaagttttttaatgac

SEQ ID NO: 4 - DNA, 2798 bp, Mus musculus
gacactctcctcctctggtctcggggtttccagagtttctccagttgcggaagacagctgttatttttctcctgaaagcttttggcacagccggcaggctgaaacttccaggcaccttttggaaaagttgttagggtttgtttgaagctttctttacattttcgtttgggttttcaagccctgactttacggaggcgagctcttcgtttgctttgaagggttcttaaagatttttttcctctccggctttcgtttttcttgaacccactcggctcaatcatggtgatgttcaagaagatcaagtcttttgaggtggtcttcaacgaccccgagaaggtgtacggcagcggggagaaggtggccggacgggtaatagtggaagtgtgtgaagttacccgagtcaaagccgtcaggatcctggcttgcggcgtggccaaggtcctgtggatgcaagggtctcagcagtgcaaacagactttggactacttgcgctatgaagacacacttctcctagaagagcagcctacaggtgagaacgagatggtgatcatgaggcctggaaacaaatatgagtacaagttcggcttcgagcttcctcaagggcccctgggaacatcctttaaaggaaaatatggttgcgtagactactgggtgaaggcttttctcgatcgccccagccagccaactcaagaggcaaagaaaaacttcgaagtgatggatctagtggatgtcaatacccctgacctaatggcaccagtgtctgccaaaaaggagaagaaagtttcctgcatgttcattcctgatggacgtgtgtcagtctctgctcgaattgacagaaaaggattctgtgaaggtgatgacatctccatccatgctgactttgagaacacgtgttcccgaatcgtggtccccaaagcggctattgtggcccgacacacttaccttgccaatggccagaccaaagtgttcactcagaagctgtcctcagtcagaggcaatcacattatctcagggacttgcgcatcgtggcgtggcaagagcctcagagtgcagaagatcagaccatccatcctgggctgcaacatcctcaaagtcgaatactccttgctgatctacgtcagtgtccctggctccaagaaagtcatccttgatctgcccctagtgattggcagcaggtctggtctgagcagccggacatccagcatggccagccggacgagctctgagatgagctggatagacctaaacatcccagataccccagaagctcctccttgctatatggacatcattcctgaagatcacagactagagagccccaccacccctctgctggacgatgtggacgactctcaagacagccctatctttatgtacgcccctgagttccagttcatgcccccacccacttacactgaggtggatccgtgcgtccttaacaacaacaacaacaacaacaacgtgcagtgagcctgcaggaaatgaagcatctgtattagcgcatttctttctgcctctctgcttgaactccagtgtttcagagactcagtctctacagcggggaacgggtacaccccagccgctgactcctcaagatgggtggcaatcagtaggcgggtctccggcttcaagtggtgcagaccagtgcccgcactgtggcataggagtgtttgctgggtggatgtcagaacactcttagaaaaattgagacctgaccactttctcggatgttggaaatgaagaacttgtttgtgttgactgagtcagggcactgctgaccttctggcgttgtctttccaaggtttttgttttaaagggacttttaaattgtctaaaatatcagtagaccatcatctgtgccatgggggacagagccaatttcaagtcatggccaaaattttgtaagaggagtgtttttgtgtgttttttaaagtcagtgttccttttttatatctttacaaagaaaagaccttccacggctggtgagcacgcagcctgtgaaattcggggcagctgctccaagttgacttcaccctgggagcagtagtagctgtgcccactgacggccataaaagccattttacagccagttgcactgtgttctcttgtaagcataatcagatgggagaatctgttatttccctgtaaccccttggaattgattctaaggtgatgttcttagcactttagcttgtcaattttgttttagtctccgttatagatgtaagctccaccagtctcttaaggattaagcccagtgacttggagggtgggggttagggtctctatccctgaacattgtagacccaggctggcctgagagatccacctgcctctgcctcctgagtgctgcgatcaaaggcccagcttggttattgcttttgaggctttctcccaacgcacagacttgtgtaattctaacactaatcctgtgaagggttgtggttgacagctggagcctgggtgacattctacattgagatgccccagcactgatcggggcacagaagcccccagaccccatttcctgtccagtgttgggagaaagtgctgctttcactgtggcctcagccctggctcggaagctcactaagccttagcactttgtcctgtgtcagctccacctgagaactgtgcagccagaatgtctgcgagctgatggaggtttcggttttgttgtttttgtattttgtgtatctttttgtatgattaaaaactatattttctacttatccaaatatattttcaccccaaagtggggttatcctttgtaaaaaaaaataaagttttttaatgac

SEQ ID NO: 5 - DNA, 1336 bp, Homo sapiens
cccctcctccctccttgcagagccggcgccggaggagacgcacgcagctgactttgtcttctccgcacgactgttacagaggtctccagagccttctctctcctgtgcaaaatggcaactcttaaggaaaaactcattgcaccagttgcggaagaagaggcaacagttccaaacaataagatcactgtagtgggtgttggacaagttggtatggcgtgtgctatcagcattctgggaaagtctctggctgatgaacttgctcttgtggatgttttggaagataagcttaaaggagaaatgatggatctgcagcatgggagcttatttcttcagacacctaaaattgtggcagataaagattattctgtgaccgccaattctaagattgtagtggtaactgcaggagtccgtcagcaagaaggggagagtcggctcaatctggtgcagagaaatgttaatgtcttcaaattcattattcctcagatcgtcaagtacagtcctgattgcatcataattgtggtttccaacccagtggacattcttacgtatgttacctggaaactaagtggattacccaaacaccgcgtgattggaagtggatgtaatctggattctgctagatttcgctaccttatggctgaaaaacttggcattcatcccagcagctgccatggatggattttgggggaacatggcgactcaagtgtggctgtgtggagtggtgtgaatgtggcaggtgtttctctccaggaattgaatccagaaatgggaactgacaatgatagtgaaaattggaaggaagtgcataagatggtggttgaaagtgcctatgaagtcatcaagctaaaaggatataccaactgggctattggattaagtgtggctgatcttattgaatccatgttgaaaaatctatccaggattcatcccgtgtcaacaatggtaaaggggatgtatggcattgagaatgaagtcttcctgagccttccatgtatcctcaatgcccggggattaaccagcgttatcaaccagaagctaaaggatgatgaggttgctcagctcaagaaaagtgcagataccctgtgggacatccagaaggacctaaaagacctgtgactagtgagctctaggctgtagaaatttaaaaactacaatgtgattaactcgagcctttagttttcatccatgtacatggatcacagtttgctttgatcttcttcaatatgtgaatttgggctcacagaatcaaagcctatgcttggtttaatgcttgcaatctgagctcttgaacaaataaaattaactattgtagtgtgaaaaaaaaaaaaaaaaaaaa

SEQ ID NO: 6 - DNA, 1558 bp, Homo sapiens
tccgcacgactgttacagaggtctccagagccttctctctcctggtaggtttcggctcaggaccctgaatcctggcccacaggcaagcctgatgggacgtgcggggtagtacttgtatggggagacgcgggaaggaggaagaagcacatctgtcctccgcggcctttagctctgagcatccggccgcgggccctgactttgccgctcagtttggggcgcagggggggccgtgccctggcggaggagcagaaggcagagggtagcagctgcggctcagcggagagacttgttgcatttgcagctaaaaccaggccctacttgtccttgtgcaaaatggcaactcttaaggaaaaactcattgcaccagttgcggaagaagaggcaacagttccaaacaataagatcactgtagtgggtgttggacaagttggtatggcgtgtgctatcagcattctgggaaagtctctggctgatgaacttgctcttgtggatgttttggaagataagcttaaaggagaaatgatggatctgcagcatgggagcttatttcttcagacacctaaaattgtggcagataaagattattctgtgaccgccaattctaagattgtagtggtaactgcaggagtccgtcagcaagaaggggagagtcggctcaatctggtgcagagaaatgttaatgtcttcaaattcattattcctcagatcgtcaagtacagtcctgattgcatcataattgtggtttccaacccagtggacattcttacgtatgttacctggaaactaagtggattacccaaacaccgcgtgattggaagtggatgtaatctggattctgctagatttcgctaccttatggctgaaaaacttggcattcatcccagcagctgccatggatggattttgggggaacatggcgactcaagtgtggctgtgtggagtggtgtgaatgtggcaggtgtttctctccaggaattgaatccagaaatgggaactgacaatgatagtgaaaattggaaggaagtgcataagatggtggttgaaagtgcctatgaagtcatcaagctaaaaggatataccaactgggctattggattaagtgtggctgatcttattgaatccatgttgaaaaatctatccaggattcatcccgtgtcaacaatggtaaaggggatgtatggcattgagaatgaagtcttcctgagccttccatgtatcctcaatgcccggggattaaccagcgttatcaaccagaagctaaaggatgatgaggttgctcagctcaagaaaagtgcagataccctgtgggacatccagaaggacctaaaagacctgtgactagtgagctctaggctgtagaaatttaaaaactacaatgtgattaactcgagcctttagttttcatccatgtacatggatcacagtttgctttgatcttcttcaatatgtgaatttgggctcacagaatcaaagcctatgcttggtttaatgcttgcaatctgagctcttgaacaaataaaattaactattgtagtgtgaaaaaaaaaaaaaaaaaaaa

SEQ ID NO: 7 - DNA, 1338 bp, Mus musculus
ctcctccttcttgtagagccggagtctgcgatccgcagagcagcagcctgctgactttgcagtggctcccctgcctcagcgcgccgcagagcctcctcttttctggacaagatggcaacccttaaggagaagctcattgcgtccgttgcagatgatgaggctgccgtcccgaacaacaagatcactgtagtgggcgttggacaagtgggtatggcatgtgccatcagcattctgggaaagtctctggctgatgaacttgccctggtggatgtgttggaagacaagctcaaaggagagatgatggacctgcagcacgggagcttgttcctccagactccgaaaattgtggccgataaagattactctgtgacagccaactctaagattgtggtggtgacggcaggagtccgccagcaggagggggagagtcggctcaacctggtgcagagaaatgtcaacgtgttcaagttcatcattcctcagatcgtcaagtacagccctgactgcaccatcatcgtggtttccaacccagtggatattctgacttacgtcacctggaaactgagcgggctacctaagcaccgtgtgattggaagcggatgcaatctggattctgctcgattccgctacctcatggcagagaagcttggcattcatcccagcagctgccacggatggatcctgggcgagcatggagactccagtgtggctgtgtggagcggggtgaatgtggcaggagtctccctccaggaactgaatccagaaatggggacagacaatgacagtgagaactggaaggaggtgcataagatggtggtggacagtgcctatgaagtcatcaagctcaaaggctacaccaactgggccatcggcctgagcgtggctgacctcatcgagtccatgctgaaaaacctctcccggattcaccccgtgtctaccatggtgaagggaatgtacggcattgagaatgaagtcttcctcagtctcccgtgcatcctcaatgctcgggggctgaccagcgtcatcaatcagaagctgaaggacgatgaggtcgctcagctcaggaagagtgcggacaccctgtgggacatccagaaagacctcaaagacctgtgactgccagtctctaggctgtagaacacaaacctccaatgtgaccatgaacctttagtcttcagccatgtatgtaggtcacagtttgcttcttccctgacatgtgatatgagctcacagatcaaagcccaggcttgtttgatgtttgcactaggagctcctgatcaaataaagttagcaattgcagcataaaaaaaaaaaaaaaaaaaaaaaaaaaaaaaaaaa

SEQ ID NO: 8 - DNA, 21782 bp, Homo sapiens
ccctcgagtctttgctcacatgtccccttctcagaggggctcaccctggtgcccttcgaaagtgggcatccactcccttcccaccctggcacccgcaccctcctgtcttcctttttctgttctccatcctactcatctcccccaactagaaaggcagctgcagctgcggagcacgggatctccatctgcagctgcatcccgggacctagaacaggtacaaaagtacctaataagtacccactgaatgaataattggctgagttttctgtaccaaagactgagctaagactctttaaaaggatgatcttatttaagccttacagcaagtaaatgttatccccatcttcctgatgaggacacagtgaccaccacgctcaaggacacagagggtggacgtgccacattcacactctgtgacttagagcggctggacgggcagggacttaggaggcctacagcagccagggtgagattatgaggctgagctgagaatatcaagactgtaccgagtagggggccttggcaagtgtggagagcccggcagctggggcagagggcggagtacggtgtgcgtttacggacctcttcaaacgaggtaggaaggtcagaagtcaaaaagggaacaaatgatgtttaaccacacaaaaatgaaaatccaatggttggatatccattccaaatacacaaaggcaacggataagtgatccgggccaggcacagaaggccatgcacccgtaggattgcactcagagctcccaaatgcataggaatagaagggtgggtgcaggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatcctcggatcctctgactctggtggggacaggcagaccaagctctcttggacccgggaagagggacccttggaagtcacttgggattgagttctagagtcttgacactgtttcagcagatctatactttgaacccacctcaggcatctcatccacagaacagggacagtgaccattccatcttgccaaggagtgcggggcacacaccatgctgctggcagccagggtggaaagtcaaggggtcccagctgaagcactgccacagaggcaggatgcagtccgagaggggacttcagagcaggggcccaaggccaaggccatcagggaggtctttctgcaggagggacctctttaggagttaggccctaaaaacaaataagaggaagaaaggcacttggtggtttttagtctattcacggtgctgtgcagccatcaccaccatccagccgcagaacttgttcatcttcccaaactgaagctctggccccgttcaaccccaactccccaccccccagcccctggcacccacccttccactgtctgtctctatggatttgaccactctaggcccttcatataagtggaattttacagtatttaccctcctgtgactggctcatttcactgagcaatgtcctcaagggtcatccatgttggagcatgtgtcaaacttcatttctttttaagggtgaatcatattccatcgtctgtatagaccacaatttgtgtagctaatcatccgttgatgtttgttgatttgtttgtttgttttgagacagtcttgccatcagagctcactgcaggctcaacctcccaggctcaagaaatcctcccgcctctctacctcccaagtagctgtgactacaggcacctgcgactgtgccctgctaatttttgtattttttgtagagacagggtctcactatgttgcccaggctagtctcgaactcctgggctcaagtgatcctctcacctcggcctcccaaagaactgggattacaggcatgagctaccatgtccagcccaattgcccattgatgggcacgggttgcttccatgtttcagctgttgtgaatcacgctgctgtgaacatgcgtgtgcaaacagcccttccagaccctgccttccattcctctgggcctatacccagcagtgcggttgctgggtcctatgggaattctacgtttaacttttggaggagctgccaaactgttttccacagtggctgcgccatcacaatccaattttaggacatttttatcacccataaagcactccctgtacccattaagaagtcatcctccatttccctccctcccctgtcctggcacccattcctctgctttgtgtgtctctggattgccctatctaagcatttcacagagatggagccatgcgctccgtggtcttttgtgtctggcttcgctcactgagcatgctgttctccaggtccatccacgttgtagcgtaggtcagcccttcattccctgttatggccaaatgatactccattgtacagacaggccactttttacccactcatctgcttctggacttttgggttgcttctaccatgtggcctgttgggaacagtgctctgtttgtattcatgtacgggtttttgtgtggacacacattttcaagtctcttgggtacacaggtgtagaagtgccagagttggggaaaaagctcacctttctaggctgtgaatgggccctggcaagtctgtggccaggactcgtcttctcttccacatggggcccctagcttggcacctagcacgtggcaggcagcgacagatgttaaaagccattcttgctatgggtagccaggctggggctccatgcagccctggccttcagcttggcagccagggcccccttgtgcctgcagcagaagccatgctgccaggagtgtaagtgtgagccaggaatgctggagaatcgtggctctgagaacagggacaagaggccacaagctcacgccttggctttcctaagcttaaggaataaacccaaaaggaggtacctggaaggagctggatttggggactgaggagctgggagctgatggaagccgtgaaaggggatgtgctcctggggaggcgctggggcgggtgggccgtggaggggacagggcccgttggttggaaactgaggcgaggctacggagttgggcactaacaggtcatccgtgcccctgcgaagcgtggggacacagggacagcagagatggcctgtctggacactctgtcgacggggggcctgtggttggtgaagcccaaggcaaggctgtgaactcagggcaagggagacgtgagcaggcgctgccgtgggctgatgtgggcactgcatgtgcaccctggcggccaaaggacctacagctcatggggggcaagggggaggagggaagccaacagcaggatgtgcgcagtcagtctgccccccctacactggaggaggagccccccggcacaaatctcgcccgtttgggcccacggacatggctggcctcgcaaggaggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagccatggcccagcagtggagcctccaaaggctcgcaggccgccatccgcaggacagctatgaggacagcacccagtccagcatcttcacctacaccaacagcaactccaccagaggtgagccagcaggcccgtggaggctgggtggctgcactgggggccaccggccacccacctgccccgcccaagggaatctctcttctgcacgtccccaccagcagagaaggctttctcccatagcttttctgatgacatgaattggggggtcctctccaaatctagaaggacaccataatatcgaatatgcattctcaagccacacaggcttcccagcccctttgagaatccgaggccggggaagagtttatgtgctctttctttgtggcccgtagatgagtgtgttcactgctagcgaatgacctctcattccacggagtccctcagcttcctggggaagagctgggtctgtctttacatttgaagccgaaaggaggcaacatactgacacacccaagggaggcgggagggtggggaagacagcagcagagggcaagaaacttctagaacttcagggtcggcaaagcctgtagcagtcattttgtcaaactccatgatggggccacttggcttttggctgcacacctctgggggaagaggctgcattggcgcccagggccatctttccattcggagccgtcctgggagagagggctcaggcccaacagaaagctgaaagctctcatcagggcagcccgagtcctgccattgggagttgcccaatccgaaagttttgcacgcaggccctcaaagaagctgaggacaccagtgaccgccccactcctggccctctccccaggtccctcctccaaaccaaattcctttggtgccttcaagaacatcgtgcaggccgggcacagtggctcacgcctgtaatcccagcactttgggaggcagaggcgggcagatgacgaggtcaacagatagagatcatcctggccaacatactgaaacctcatgtctactaaaaatgcaaaaattagctgggcttggtggcgcatgcctgtagtcccagctactcaggaggctgaggcaggagaatcgcttgaacccgggaggtggaggttgcagtgagccgagatcacgccactatgctccagcctggccacagagtgagactcttgtctcaaaacaaaacaaaacaaaaaacaacaacatcgtgcaggctgtggtttccagaagccacgccagctccttgattgccaataaacatcccgctgtggggtggccaggaccgagtgccaattagtgacagagtgcccagaccaaaccggatgaggatcttgcagttgacctcaacatgactgtgcccagaatttccttggtggcaatgtcaacagtctcttcctagatgcccccagacttcatcaatgcatgatgcttcagtgcactcttttcaaatgtcggggtgggttttttttttttccacaaaacttcaagcatctactaaagtagagggaggagtgtaatgaactccggtacccatcactcagcttccacggtttcatctcatttcatctgtgacccctccactaccctttcttcctgattcttggaagcaaatccaagacatcacacccttccctctgtaaatctttactatgttcctctaggagaaaagggctcttctcaatacataaccacaagtcatcatcacaccgacaagtgtaacagtatttcctgaatagcttcaaatatcctagtagtgttcaaaaaatgtcatacgtattttcagtctgcttgaatcagggctcaaataaggtccacacattcagattgactgatatgccttttgactacctttgaatctagaggttccctttctatctccctgcaatttatttgtggaagcaagcaagtcgttcatgacgtagcctaacaggcccctctgacgttgttcattatgatttttctgtaaattggtagttgatctgaggatctggccagaggtaggttggatttgttggtgtgttttggcaaggagagtgtctcttttctggggtgttggcagctactgaaactcaatgcccagaccaattaaaccactggggatggaaaatgacggcattcggacaccttaccctgccttcacctattggtgaccaaaaccttaacatcttcacaggtcttcttaccctgagggtatatgccactaggttgtgtagtaaaccggtgtgtttccagtcccttagaatagtccctctctaagtgatatgccactcagtggatatgcatttagcttcatttcttttgttgctgattttcagagattgctctgtaaatttaaacttttattttactttattttattttttcgagacagtcttactctgtcgcccaggctggagtgcagtggcgcgatctcagctcactgcaacttccgcctcctcggttcgagcgattctcctgcctcagcctcccgaggagctgggactacaggtgcccgccaccacgcccagctaattttttttatttttagtagagacagggtttcaccatgttggccaggctggtctccaactgctgacctcaagtcgtccgcccacctcggcttcccaaagtgctgggattacaggtgtgagccaccgcccccggccacttaaattttgttttataattatgtaataaaacagttaaaagtctcaaattaaaatctagaaaagaaggtgtatttgaagaagtctggcttctctgcgccaccaccgaccgccccttccctacctgcctgtatttcctcgaatcactttgcctgggagttgactttgattctcttgctcattgcttcatgaaattcagttccagaactttcaggagggaggggtaggccatgacaccagctctagttacactggtggcagctcctgtcccctcccccactgctgctgggacctgttctctcctttgcccccttgtccctgcactgcccaatttggaccgcaagggttgccagggaagggcactggctgccttgttttcagaggtcgtagcacctagattgctccagccccttgcacttgcctgcaggccagagtgtcccaaaccctcccagtctcagctgctcttccccagttcacccaaggtacttcccagggaagagctgccgacagtttgggggttctctgttcttaggtccatcagcaaccccattgctcccctctgcttccttctgcacggagactgacgccatgcaggtcttcaattgtcaatggtctgtccctgctgctcatactgggggttcctggggagccagtgccaggtatcgggattgcagacattgtctgtgggtttccagaagctccttgtgttaggaacatatggggcccgtgcacagagggcagcagaggccttgtgggatccagctgtgctaggggtgagatttatctgtctctcctggccatagccaggaaatccccatttttcttaagctagcttgagttgggcttttctaacacacagctaaagaatctcttgataaaccttgggactctccatgaggccttatatggcagcaggtctgtggcttgcaatcccttcaagtaatctgccaaaaacaatgttatgacgaaggtccttccaacacaaaaggtgtagagccctagcaaactcctacagaagaaaaaggagaaataattcgtttgtagtcccagctacttgggaggccaaggtgggaggatcacttgaagtcaggagttcgagaccagcctaggcaacatagccagaccccatctctacagaaataaaaaaaattgccattgtggtaatgcacggcttgtagtcccagatactcgagaggctgaggcaggaggatcgcttgagcccaggaggatcgcttgagcccaggagttccacgttgcagtgagctatgattgtgccactgtactccagcctgggtgccagagccaggctctatctctatttggtgttgttgttgttgctgttgttgtttttgagacggagtcttgttctgtcaccccggctggagtgcagtggcgtgatctcagctcaccgcaacctctgcctcctgggttcaagtgattctcccgcctcagcctcctgagtagctgggactacaggcgcccaccaccatacctagctaatttttttttcttttgtatttttagtagagacggggtttcaccatggccaggctggttttgaactcctgacctcaagtgatccaccccctcggcctcccaaagtgctgggattacaggcgtgagccaccgctcccagccgactgtatctctaaatatataataatcataatcataatcaggacagccgtcatattggattagggcccaccctaatgacctcatttaaacttggtcatttctgtaaagaccctatctccgaaaacggtcacattctgaggtattggggttaggactccaacatatgaatttgggtggggacacaattcaactcataacacattcattgattaatttcctcattcatttattttctgagcatctattgtgtgctggacactctgtgaggatgaatgagtcaaagtctctggtgagaaagacaagacttgtacttgtgtctcagtactggctgctataagaaattaccaggctgtggccgggcgcagtggctcacgcctgtaatcccagcactttgggaggccgaggcaggcggatcacgaggtcaggagattgagatcatcctagctaacatggtgaagccccgtctctactaaaaatagaaaaaattagccaggcgtggtggcgggcgcctgtagtcccagctactcaggaggctgaagcgggagaatggcgtgaacctgggaggcggagcttgcagtgagccgagatcgtgccactgcactccagcctgggtgacagagcaagactccgtctcaaaaaaaaagaaaaaaaaaagaaaagaaaaataaattaccaggctgtgcatggtggctcatgcctgcaatcccagtactttgggaggacaaggcaggaggatctcttgaggtcaggagtttgagaccagcctggtcaacatggtgaaaccccatgtctactaaaagcacaaaaattagctgggtgtggtggtgggtgcctgtaaatcccagctactactccggaggctgaggcaggagaattgcttgaacccaggaggcggaggttgcagtgacccgagatcacatcactgcactccagcctgggtgacagagcaagactttgtcccaaaaaaaaaaaaaagaaaagaaagaaaggaaaagaataaaagagaaattaccatagattgggtggcttttaaatgataaatttatttctcacagctctggaggctggaagtcagggtgctagcgtggtgggctctggcgaggaccctcttcctgactgcagattgccaacaactcattgtatcctcacatggaagaaagagagctagagagcactctagggactctttttcttgtttgttttaattaaaaaaaaattttttttacatgggcatgccatgttgcccaggttggatttgaactcctgggctcaagcaaccctccagcctcagcctcccaaagtgctgggattacaggcatgagccaccattcccagctaatttgggctgttcccaaaggctcaagtgatcctcccacgttggcctcctgagtagctggggctacaggcgtgagccaccatgcccagcttctaggacctcttttataagggcactaatcccattcatgagggccccactcactctgcacacatgacctaaatgacctgccaaaggccccacctcctaataccatcaccttgggggttgggatttcaacacagaaatttatggggggcacgtacattcagatcatcatgaacagtaactcctatgtgtgacagaaggtgacagaggtgggtagtggtcttcccctcaagggggtgagttgccactagctggggaatcttctggaaggcaaatgcatatgagctgggctttacaggaggcaagcgtttctctatggaagggcagaggactgtgggaggtaggaggtggggctggggcaaagggaagaggggagcagggaagtggggtgactgcacactgggagtggggaatcagatggaggagacgatgaggagttctgttaagttcaagatgccagtgccagtgaccagcgggcgatggtctctggcttgagggacaggatggaggggagactgtctgaggatggacaaagctggagggaaacagccaattgcaaaggcaggagggcggaagggggaggggagaggtgggatcagcactggtatagacaggcggtgctgcagcccagctcctctctctcctctgcctcctgccctcaggccccttcgaaggcccgaattaccacatcgctcccagatgggtgtaccacctcaccagtgtctggatgatctttgtggtcactgcatccgtcttcacaaatgggcttgtgctggcggccaccatgaagttcaagaagctgcgccacccgctgaactggatcctggtgaacctggcggtcgctgacctagcagagaccgtcatcgccagcactatcagcattgtgaaccaggtctctggctacttcgtgctgggccaccctatgtgtgtcctggagggctacaccgtctccctgtgtggtaagccagtcggggcccaggctcggcggaaaccactcattcaccctgcaagctcctccagccacctcatgatgatcggggcccagctgctcctgtaggcctgtctccctccccatctgcgcctcacatccatatactgaagggttctggaggcttccatctgaacactcacattaaattcagctcccttgagtcaaacataccctgagttcctactcttgagtcaggctctgcccggggacagccagtttggagctgtggggctggtgtgggaggagacagatacagagctagacaaccccagaacagtaggggggcggggactctgggcaccctggacagaactcccctgcaattagggatgcctgctctttcagctcgccagcatctgcttttcccggaggagacacaattcccagatcctctccccatccccatcactaatatctctgtgggccactattccgctcaggtcaggagacagtggccgagaggtactagcgtgccaggctctgtgctaaggagggggccctatagccagacggcaaccacacagtaccatcatcagtcctctcagacaagaaggggcctggggcaggtggtggaggagcggctgggagcagtttgtggttcgagtggatagagtaccaccaagcagccgtggctgctggacacgaggtgggcaggcccaggtctcagaggcctcagacgtcatgcccaggagctgggactttctttcaggaggaggagaccccacatccagcagcagcagctcctgctcttgcctccccaccactcttagcagcctccccaaccccaccccgttaactgcctcaaattgtacccacgatggcccagaccagagagggtgcttgtccaagtcccggcactaccccgatagtgtagaaggggagccaagggaaggtcaggcagagaaggtccatccccaggtccgagtgctctctgcagcaggcatggcctcggtggtcacacgacccttcccgagtgcccccctgcatctccgcccacgtctgtctccgtttctgccatggtctcccgctcacccttgcctctgctcatggtctgttcttgggtcagtcaggtgccaagcagccagcacttccccaccacttttggtccacggatgcccttggccatctgggaagcctgtggaccccatctcaggagaatttttgcaaacgcataaaatgagacccataggattacaaaggcagcaaattatactgaaatacagttatcaaagtattaaacattcatcagtaacatagtctttagttaaaagcatttactggccaggctcatacctgtaatcccagcactttgggaggctgaggtgggaggactgcttgcctccaagagtttgagaccagcctgggcaacatagtgagacctcttctctacaacaaataaaaacagctgggcgtggtggcacaccagtagtcccagctactcaggaggctcaggcgggaggatcgcttgagctctggaggtcaaggctgcagtgagctatgatggcaccactgcactcagcctgggcaacagagtgagattctgtctcaaaaagtaaataaaaataaaagcatgtgttaaacgtattagtgacaccactcagtattaaggtattaaataacaggatcccgcctgacaaccactgttatttcagagtagtgatgaacataagtggtattcgaactctctgccacctctatgaattgacaggaaaacatctgtgacctctcttgctgaccgagtcacgggtactgctaatactgccacgttcataatggaaggaaatgcccagtgtctgttcgaggttggtggaaagaaagatgtcgttttttccacctcagtccgtggagccctgaattctgtgtgcagacgtttggggtctaagcaggacagtgggaagctttgcttcccacctttgctttggctcaaagccctcatctgtctgctctccccatagggatcacaggtctctggtctctggccatcatttcctgggagagatggatggtggtctgcaagccctttggcaatgtgagatttgatgccaagctggccatcgtgggcattgccttctcctggatctgggctgctgtgtggacagccccgcccatctttggttggagcaggtaagggtgcgaggacgcaagatggagtgggcagggtcagactctgtgaccttaaggcaaatcacttcctttctctgggcccctctgagcgtgcaatgtctatcaatgtatgaatgtggctgcaacataggaaaggctctgtggtccccgaacctctggaaacatatttatcccaagcacgatcaggtcacaggcgcacacggagctcaggccatcagcacagctgtcagtgaacgcatagcgtgtttgcattccaggtctctttcttgcacacgctgccgcaccacgccccccacctttcagaggctgcttgggtcatagatccacctgggcctacagagcacatgtcctggccaggccaagcaagtggctcaaatgtttgattggagtggactgggtgggacagcatttcactgttttatcgacaagctcgtgaataagttctcgtggtgtttggagagggaatgttctttcctcgagaacgttccacaattctaggaaacaaaccttgtggaagcctgtctctgtctcccgccctcctcatgccgccatgccccacacagctgcccgttatcaaacatgtgtggtgagctgaccctggtggaggctctcccgcgggttatctcatttaatcctccaggccactaagtgagcagggccctttatttcagtcatggcctagctgacctcagataaaagactcagctcttcatgggtgttctcagaaggtcagggcaagaaggaacctcacaatccctttgtaaagaaggggagtgattgggaagatgaaaatgtcctggaagcagatagtggagatggttgcacagcattgtgaatgtaccaaaggtcacaatggtacttttttctttttttgagacagggtctcactctgtcactcaggctggcacagtgcagtggtgtaattatggctcactgcagcctccacctcctgggctcaagtgatcctcctacctcagcctcctgaggagctgggcctacagatgcaccacttcacctagctaattttttttattttttgtagagacaagatctcactgtgttccaggctagtcttgaactcctgggctcgagcaatcctcctacctctgcctccaaatgtgctgggactataggcgtgagccattgtgcctggcctataatggtacattttatgttatgtgtattttaccacaattcaaaaagaagaaaggcatgacatctaaaaatggacaaggattaaccaaaatcctacccaacggttttgttttgggttgatgaaaatgttctggaagcagaggtggtgactgccacagaattgatcacttcaaattgggtaatctcatgcaacatgaatttcacctcaatttaaaaaaacaaaccccacccgagttagcaccgtgcctgggccgggggtcctgggtcaccccaccctgcatcaggactggctgccggcccttctctccaggtactggccccacggcctgaagacttcatgcggcccagacgtgttcagcggcagctcgtaccccggggtgcagtcttacatgattgtcctcatggtcacctgctgcatcatcccactcgctatcatcatgctctgctacctccaagtgtggctggccatccgagcggtaagccccccgattcctcctggcctcacccgcctcctgcccctaagctgctctgccctcaaatgagtccactgagactcctaaactatttttccaaaaatccttagagaagaggattttacccctataagaaaatattaagatccagcgatgagaatcaggtgattcctttgggactgtaccagtggctgcaggttcagccccagccccgttgtcctcagctctgtgagacgggaaagcactgccactccctccctggaggagtccactaagggaacagaggtgtgccttgccccgaccctggacagttctccccggggtggaaaggctgcctttcccacagagtagagtggagcagccacatcagcaaatgacacctgcaaatcaaggcgtgtttttatgaggctgccaccggagtacccttgtccttttcataggctgtggggccgaccaaggagtggacccgagagtgccatttgcccccctgacccactctccacctccatgtctggccctctgccctgggaagctgatcctgtccacagccgtcaccccccacccctagactaggctaccactgggagcccttcaggaagtcagagcaagggaggagagccaggctggttcttttctgttagcagtgggagccctttcagggtgctggctttcctatatgaagctgcctgtgcccacaattggatgggcatgcctgccaagctctctctagaggagtctgtgagcctgtgaaaggccccctcaccccgtcaccttggggtgaaggctcccacaggtacccaaccatggcttcggctgtattagtctgggatggtagagccccagctccacaatgtggccccagctctgctgtctcagccatccctgcattccagccctcacactccctctctcatccccactcatctgcctgccgccagtccctcatccctggcaggtggtggctggcctctggcctcccccacagtgcctctgcctggaggccattcgtctccttcctcccagcaggcatgaaggagccaccccaccaaagctgccctcagctgcctcaccgtgagtccagggcaggatttagtccacagagtggccaacctggcctaggaagcctgagggaagtgtatgcattgctctgacactcccatcgcgcaccccgccagccactgcttttgcctcccccgccatctccaccttgttaactccttcattctccacgcccagtcatcaatcaaatcaggcctccatgctcaggcctgagcgcaggacaggacagtctgttaagggatcaggtgaagcaaaggagcttgttagatccagctctggggtcatcttaggccacacctagctgcatgccacctccaattctagaactcccccagggccagcctgaggcagccatgtctgcctggggccggctgtgctccactcagggctggaagatggctgctgggctcctctcctcctccccacaactccctatgcctgggtcacctgcctcttgctgccctccaacccccgactcactatccctgtctcccttaggtggcaaagcagcagaaagagtctgaatccacccagaaggcagagaaggaagtgacgcgcatggtggtggtgatgatctttgcgtactgcgtctgctggggaccctacaccttcttcgcatgctttgctgctgccaaccctggttacgccttccaccctttgatggctgccctgccggcctactttgccaaaagtgccactatctacaaccccgttatctatgtctttatgaaccggcaggtaagcaacaccatcagcagatcccactcaaaataccgtgtgccctagaagggtgcagtgatggccccacctggaatcatgtctctgataagaagcccgcggagcatctgggggaccctccagggaaatgaccgggaaaggctcagcgtgtgacccagccccagccagagctccagctggcccttagcagaaggcttaggtgtgccctctggaatcctttatagtctcggcctgagggtggcatttcccaaagcgtctgtgtgccgtgcgctcttcccttccggtggccctagaactatggctgccgagcttcaggggctctcctggcgttcagacgctctaggagttggtgagccctaggtacatccaccctaggtgtgcccctcttctgttcagactcgacccttctcaaccttcatctctccattttcaaaccgtaacctctggaatttgtcttcctataagaacaaaagccggccctccttggctacactgaccaagagttcaagagctttcacgagttcgtgggttagttcaggggggacgtgctgtggtcctgcccagaggcagcctccttagctggcatattgggcctcagcagcaagctgctcacacacctaaatccccccacctcctgcaggttacaggcttcattaaagcgcagctgtgatgtgacttgatggtggccagaaaggtgtgcagaggcctcccatttcaccaggcccagtccatcccttccactgggctcttccttgcttctccatcttagagccactcaatggctccagcccctttggctcagctttgactcacacaagccaagtctgcagagttcattaagggttcattctctctggtaacttttaaacagtaagtaggaccaggcctgcagtggatttccgggaactcgctgtagcacactgatgcccagagtgtagttctatccctgacccctgtttcctgactttcatgaggatcttttttaggtttctggaatcctaaactatcttgccaagtactgtctttactggattatttccattctcctttccagaactccccctggacagggggagacagatgtctgcacttctggacctcaccaggcctcgaactttgcttttaccctttccacataattatcctgtcctgccacattctgagagaattttctggaacgcagttccatgaagacagcaaattttgctcaggacagagtctggcacacagtgggtgctcaagcagcagctgctgaatggattcctcagccctatctcccagctcttcagccgagctgattctgctgtttgtcccgtttcttatgttattaatttcaaccattatattttttatttttgagagttttgatgatagagggagttagagctagtcaagagtaggcctgaaatatttagaaaatgcctttggtctgggtcctcaaagcattgtggttacttcagggatgacacaggacatgatttgagacattcatatggcccagatctctttggggtgaagcagcaaagacagacccctcctggtaccggaagacgcttggctggagagatgaggtaggggctagattgtcattacctaggcctcaccttgccccagatccatggactggaaaaaacatgacaaccacatgccttttcattaatattcctccgagccgctcaccagacagtctggggacaggtcaccactgccccttagctgtcactgtggatgagtgtcatggggctgccgtcacaaactaccacaaactcagtggcttcaaaccacagaaatggattctctcagggttctggaaatcttgagtctgaaatcagggtgttggcaaatggaaaggttccctatggaggccgggagggagaagcagctgcagggctgccggcagtctttggcgttccttgactccaaggtgtgtcaccccagtctctgccttcatcttcacgtggccttcttccctctgtctgcgtgtccgtgtccaagcgttccttttcttatcaggacaccagtcattcgattagggcccaccctgctccagtgtgacctcatcttaacctgaacacatcttttgggggacccacttcaacccagtgtagtcaccatcaactgctaagtcagatgacatccccgcgtgtgagggagaaataatccaagccttcctccatcccccatgggattcggaatgggtgaagggaaggctcgggcacgtacattcagcacagtgctccacccttccctgctctgctcaataacgctttctgtccttccagtttcgaaactgcatcttgcagcttttcgggaagaaggttgacgatggctctgaactctccagcgcctccaaaacggaggtctcatctgtgtcctcggtatcgcctgcatgaggtctgcctcctacccatcccgcccaccggggctttggccacctctcctttccccctccttctccatccctgtaaaataaatgtaatttatctttgccaaaaccaacaaagtcacagaggctttcactgcagtgtgggaccacctgagcctctgcgtgtgcaggcactgggtctcgagagggtgcaagggggataaagaggagagagcgcttcatagactttaagttttcccgagcctcatgtctaccgatggcgtgaaaggatcctggcaaaacagaagtgtgaggcaggtgggcgtctatatccatttcaccaggctggtggttacataatcggcaagcaagagctgtggaggggcttgctggatgccctcagcacccaggaggagggagggagctagcaagctaaggcaggtggccctcctggccccttaaggtccatctgctggaggcccagagtccttggagtacagtctacacctggaggggacccattcctgccagtctgtggcagggatggcgcgccacctctgccaggccaggaccccaagcccgatcagcatcagcatggtgcaggtgcacaggcgtgagctgatcagtgacgaggggcaggcacacaaggtggagacaaagaccaagaggacggttgccagtgagaggcgcggactcaggaacttgaacaacatctgcgggggacggctttggaggtgctccgctgcctccagttgggtgacttgctgtagcatctccagcttggatattcggctcttgaaggtctccgtgatctcctgcaggagacgaaaatgcacgcaccagaagtcagcacagagttgtggtcgtttattgagttcttaggggtgagcagaaagcactgtggagtgggtattcgaggagggaagcagagagcctagagcacattcagggcagaggggagggcgcaggctctccagcaacagggaaagcttcatctgacccggctgcactcccccatccactgtctcccgaagctgaggacctggtcaagacacagctacccagggacgggggtgggcgctatgggaatggaaaagtgaggagagggaagccaggtctaaggaggggttctgagagggcgctccctacacctgcagccgcagcagaagcagctccaccccagatctcccgagtcagaggctcacgggtgagcactgcagcaccagagtggcaaaagcagctaagccagatggtgggaagcggagcgtgagtgtaaagatcagatgctgctagctctgaaacaaatgtgtgtggccatcgaaccctcaggagggggcagctcgaggacccgtgtcttgctttggtttgggggtatcagaatagattcgctcatccctccagtcttcttgcaaggctcccccaggaggttctcacccatatttccttggctctctcataggatagataggccattctctcttcgctgcaggccagattgtgtttgaggttgtaaatctcattcagctgtccctgcaggcgaccattcacctgttcttccatcaatgcttgccttgggagcaaaagagaaagtgagattccttcagtacctcatccagagctcatgccaacagcgagcggtcctgacctagactagattcgggttcagcttctgccttcctccccgctccccaggctctagggaaagcctgcctcccactccaggtctgcctgggaacaccccaaacacacaccagccacacgcacaccaatattcatatatattttatatgtagttactcttttgtaacagctttacaaaaatagccacagactagaagttgtagaaatgacagtgtgaactatctgtgtgtgctcttcagttccgcaaatcccccaggaccagtgttgagcacgaagtctagcctgcagtttagcctgcagttactctatagaaacatggtgactataaggattgaaaagccacaaaagaccacagattccagtacaattccattagttacgaaatgtccagaataggcaaatccatagagacagaaagcagattagtgggtgccaggggctgggggtgggggatgggcagtggctgctaatgagtacagggttgctttggggc

SEQ ID NO: 9 - DNA, 17024 bp, Homo sapiens
gtcggactcccgcggccggactgtgggggagtcacgcaacaggtgagaccccaccaaaatctcgccggctccgcgccaggctctgcggcccttggccccgcccccgcctggccccgcctccgcctgccgaccccgcctcgccatttttttctcttcctgggcgccacgtgggcagccaccacaccgcggtagggctggcctgagccgaccctgggtcttgggccagttccgtgcgtcagcttcctcatcagtaagatggggtaatgacagtacctcactcaaaggggttactgtgaagattaaacgtaaagtccctttacacagtgcctggcacgaagtgagggcccagtgaaaggtagctgttatcattgtcccaggattcattttaagcagaagtcctgcccctgcaatctcacttggtcgtctgttttcttccggccggcgtaggtaggaggctgtgccctgcaattctggctccccttcccacatttctgtatttggtggttaagagctttaactctagccagaatgtctgggttcaaatccagatttcacctacttcctgactgatactgggcaagttacctaacttctctgtgcttctatttcgtctgtaaaatgagtgtaagagtacctgctttatatggtggttaagataaatgagttaatataagttaactgcttagaactgtgcctggcctgtagttagtactcagtattcatgtattaccattgctttagccttggccttggttctctattgacactcacaggttgattcacttgcagccgttctggggtctatccaacaagcatccccactgccaaggggtagataatccgacctaaagggaacttgtggagggaagtccctaagacttcactgaatataattaaatgcattagcaggaattaatctcttccctattttccctcttgtgggaggcctttgccccccacacccagttgttctaatgctgtttgtctggctcctcccagttcttaccactcccacagcagagatccttcccaaactctgcctccctcttctctccccacgctggattctggaccctgaacccttttctccacctgcatctggtgccacttccattgatccctggctactgttccctgaacaaacttaaaaaaaacttggcctcttcacctcacacagaatccataaaactaaaaagagaacagagataaccaggtttacctaatcaaaatagcaaagactaaaaaacaaaaaaccaaaaaataaacgctaatgcaggttggcaagggtatagttagtgagatgggagacacacacacacgtacatgtgcacaaacacattactggtggccttgaacactgatataatctttctgaaagccatctggtaatatagaatcaaaattttcaaaaaggttcttattatttgacccagtaatcaactttctggaagttgtttttctaaaaagagctgccagaaaaatatgtatgcaaaaagttgttcagcaccacaatgtgtattgcatagaaaagttagatactttaaaggtcgaacaataggggaatgtttatgtaatattcataccatgaaatacagccattaaaaattgttcttggagaaatgtttaatgatgtggaaatgtttacaatataaagttaggtaaaagaatggatgcaaaacactatacacagtatggtcctacttatgtgatatacatgggtagaaaagaaacacaccaacaagtttaccaatgattatctgtagtgatggaattatgggttatatttattttctttatatttatttgcattttagaaattataatacatacccataacttttataatcaaggggatagaaatcaacaaaaatctgtaaaaatctgtaattgcctccacagtccccccaacccagccttcccggggtcttaaccccagacttaaactattctgtttacatttatggcccactgaattggtcttggggcgcttagggattttgaggctgatggactctcaagaatgaataaaatgaatctcaaaaatcaagcaccatgtaactacataaatgtcatgtatcttctgataagatgcaaatgttgacatagttttaaagatagtatcttgcattgggccaggtctcttgagcatcctgcttaatgcagacctacctacctcagcagccaataggtggcaatccttgccctccacccagcccaggcaaaccagttcattagtccctttatgctagtcccctagtcccatctgcaacacacccttcactctacaaccctcccctatttccaggtcatctggtgtattgttgctgctaggaagtcggccctgattatagttaggcatcaatccctccacccactccataccagcaagaatctcagtgaggtttcattgtctacgcagaacagagcttgggaaataaaggagtgagtcatcctatggcctgggcttttttcacactgaggccttaacattcattgcctttgagcctctgagatattccctagctccaaacacagcttcctcccctcctagcctgttccccacatggctccttaactggccctcccggaatcccagctccctaaagatggtgcactcctgtttctctgaagaagtgcttttctttgcttctggacttcagctcacttgtgatgtccgcctccctagctaggccagaacaatcttcacagatactaaacttgggagagaatatctgcagctccaaaaaggtccttggccaaggcaatttcccaaaccataccttatgcctaggcccttctagaaacaagctgttcagctctggtttctcagcttctcccagcagccagctccctctccccctcacctcagtcacccagcattgtgtttatagcaccatacagctgtttccatctatacttgtttataagaccccagtggattttagtcaaaccccttgccattgaaggcgcttcactggctggctccaatttccctttcttgtcttcatcttcacctcccaaccctccactccatgcacctttcactccagtccagtcacatcaaagtcccagtcttctctgagcacaccttgtactttgtgcctctacctcttttttcatgctctttcctttccccaaaatgcgtatctcccacttccactgttattctgcttctctgtggaaagtctattcatttcacaatcacccttttctttcttccgctgtagaactttgtcagtctgacagtcagatgtgtgtttgattgcctttcctaccaggggtgaatgctaggtcttaaacagctctgaatttcccagcactactgcagtatccctttaactcatttgtttttctgtgaatttcccccacctctgcctatggactggaaggtcctgaaggtagttcctcatccttcatgtcctcccactccatctgggacaagtctttacccacaattcacttaagacaaggcttaccacggatgtcagacagatcccttaactcaggaaacatgcccaaatcactaagaaacagtgatttacttttcattcaaaatgataatggttataaagggctgtgaatcaaaacacctgagctctggcttggcttctgataccaacttgcttattaatgtgaggatcacttttctgtacttcagttcactcacctccaaaatggaactagcactacataccttatttacttcatcaggaaactgttcgtaaatgtgatcataaagtgggacagccacaggaaaggcacaggaactgacgatagaggcagaggcaggaggtcacagacatcaggatgatgtatgcacagtaatggatctgtactctcaggccaggcgagggctagcagttccctctctcagcttagcagtgagaaaggggaacttccatccagtaagttagggaacaaagcagaagcagcacagaagtttgcagaggtagtagcagaaaaaggaacaagcccactttccctgtgtgtgaaacaacttagtggttcctttaacaagggctgctgttatagagttcatgcatttgtccatgctttctctctctggctacttatgtggaagtaatagaaagaaggatcaccggtttgtctgtttactaggccatataactttgtttttttgtttttgaaacagagtctcactctgtcacccaggttggagtgcagtggcgcaatctcggctcactgcaacctccacctcccgggttcaagcgattctcatgcctctatctcccaagtagctgggtgcatgccaccatgctgggctttttcttttcttttcttttcttttttttttttttttctgagacggaatcttgctctgttgcccaggctggagtgcagtgcctcccgggttcaagtgattctcctgcctcagcctcccaagtagctgggattacaggtgcctgccaccatgcctggctaatttttgtattttttttaagtagagacagggtttcaccatgttgaccaggctgtctcgaactcctgacctcagatgatctgcccattttggcctcccaaagtgttggaattacaggcgtgagccaccatgcccggcctaatttttgtatttttagtggatacgggatttcaccatgttggccaggaactcctgacctcaggtcatccacccacctcagcctcccaaagtgctgggattacaggcatgcgccactatgcccagctgactttaaatgagttacttaactctcaagtctcagttaatctagtctgtaaattggggacaatagcagttattgtgataattaactggtatgagccacttacattgttcagcagttattaattataataactataacataataattattaatagttccaggcagccccttgaacagtgtagccttcagcatggctctggcctcagtgtgagttaccagtattgtgagtacgctggagcagggctagcagtggggtataattactgaaaccttataaccaagattccaagatagtcacaatttcaaagagactctcctgtttttccttaagtcaataaggacttgccaatttgattgggaaaacatggctaccatggaaagcacctcagcagctgcatatttctccatcagcccctacagtcactgaaaggtggtgcgaaggaggaagataattagctatggctcagggtacctgataggcggggagacctagattctactcctgaccttcccagtcccaactggccactgctgcacaagtccagctctaaaatgaaagagcaaattacatccttcgacctcagtagttctccaccttggctgtacactagaatcagctggggacgtttttaaatccttgtgcctggactgcactccagaccaattaaatcagtattttttaaaaagctctccaggtaaatgccactgtacactaaaagttgagaactgtttcaggccagggtttttcaaacttcacagatgtgataactacctgtattagcttaaaatattttctctggctctggagtttgatgctaacataccatttaaaaacatacactattttaacatctcggaaagcagggtaatcttctagtcagtggcacctaagacttgaggaaacacagacaccagcaacccaggtgattcttaggattaggtaagtttagcggaaaacaggtccctagggcagtacttcccaatctttaatgtctttagagtcacctgagatcttgttaaactgcggattctgaatcagaagttctggattaaagcctgagattctgcatgtctaatgagcttccgggtgatctgatgttgctggctcttgatccacattttgggcaacaaaattctaaaacatctccacctgaggaggctccgccagcaacactcatttaagtaaactgaaataattggcatagaggagtacaacctgtggagtcctaaatgtctgtgaggcagttgctagactgcaccctcatttttcccccaggaaagggcagctgggatgagagccagaaggagagagagctgccccaacctttgagaagccagagtctggagtccaatttcccaaagaagcagagttttttgtgtgaggcagcacaaaccccacactgaataccagcaaagttcatttatgaagtgaagttgggactcagctggctttagtgggccaaagggaagcaaccccattctcttcaccatacacccttttcctgcatttattcattcaccaaaccttttgatccacaaataaactacaagttctgagaggttcaaagatgagtcagatttggtccgttgagctccagggggagaaatgcagtgagggaaaagatttgtaaaacgacgtacaatatgaagtgataagtgctaataacagaagtacaaagagaggggccaatgcaaaggaacaatgctagtagcttcctggagcagcagccactgcgggagctgaaacattcctaatcttcccaaggaagggcaccacccaaaacaaatttcctggccaggaccagcctatggtaaacgagtatgctttgataccctgaagcccttgagatcaagaccttataatctggaggctcaacataaggaatgctttctacatatgtgccagtaatctctagctctatgatgcaaataaatctaaggaagcaagagactttcaggggatgaaccccttaaaggatggaagtagtcgtgcatcctatccttccgtcagaacccagcagatcatttccctagttatagaaacatttgagtctttaccccttgccatattgacaaagctcttaattggcttgacctatcacattgctagatataaaggctacaatccctagactaagaagtaggtctccagttgaagtagggagtctcagtcaatgtaggcagagtacaagaccctacagcctgctctctcacctgccatcgtacagaccagcttttaggggagccaagttgggatactcaatcccaacttttttccttctcttccatctcacatacaggaaaccttacgagagaggattaggggcctgaaaaagctgacaagacggcaaatatgggaagtggagccagtgctgaggacaaagaactggccaagaggtccaaggagctagaaaagaagctgcaggaggatgctgataaggaagccaagactgtcaagctgctactgctgggtgagtgagatgggaagatgagccagagaaggcaggggtccttcctactttcctgaagggttggtgggttctacctcaccccatgggaaggaagggtggcaggtcatttttcctctcctctacacagtctggctaggggatcaggagatctagagctgagttaatatggggcctaaacagccaccccaaggggatccagaatgccaaggctattccagagtttttctactcttgagcgaggaatagtgcaagggccaccatgaacccacccatttccaaatctaaccaaacctaacacatccttttggcttcaaggaccctggacttgcagactgtacccaatctgcagagaactctaagccaagaaatcagaagagaacaggaccttccctcaccaacaggctcacaagtcccaccatacagtcagtgccaacagtaccagagatagtcccactggtttttgccaagtatagtggttccttcttgctttcagtaaaaactttggaagtagggggctctgaggaaggaggaatggtgtctttatgtacagcagtcccttcctggctctctattcaatagctgcctgcaaagctcctgccagatggaaggttcatcaacttgatgagctcctaagcagatcactggtctgtgctgagaaaataaaagcacctcaatttgtcagggaaattgatcacagctgtaaataaaaccaagacaagaacatttgagacacgtggcttaggaaaacaaaccactggtaccacagaagtagggtagctggagcaggtagggtctacgtagcagaagattagatgcctgagctgggtttccaagcccccataagggatctgggagctgacgcactaggctaaggcaccttcttttcccccagctgatctgtggcacagtcgtaaggacacactaaaggagcatatctttgtaagctggaccagactctaaggagcccaggaggttacgcagggggaaacagagatggtggggccactgagagatcttttaagcctaagcagatttcttctacattcaggataagctgcttagagggaacaagcacaagcgaaataggaggagttcgaggcattagggtagtataaactcagtactagaaggtatgagtttttgatggagagagcagagtgtgaatgaggacattaggacacattagtcaataaagggaacccacttagccccatccaagaccaggttgagcaacatggtgaaaacctgcctctacggaggggcggggggaaatggccggatgcagtggcgcacgcctgtaatcccagcgctttgggaggccaagatgggtggatctacctgaggtcaggagtttgagaccagcctggccaacatagtgaaaccccatttctactaaaaatacaaaaattagtcaggcatggtggcatgcgcctgtagagtcccagctactcaggaggctgaggcagaagaattgcttaaaacccaggaggcagaggttgcagtgagccaagatagcaccactgcactccagcctgggagacagagcaagactgtctcaaaaaaaaaaaaaaaaatgtagtcgggcatgatgtcgcacacctggaatcccaactactcaggagactgaggtgggaggatcacttgagcctgggaagtcaaggctgcagtaagccgagattgtgccattacactccagcctgggctacaaacctgagaccctgtctcaaaaaaaaaaaagggaaccagcaaggtgatgaaaatatagttattttggtgaaatgatccagctctctccaatcctaccccaagatcactctcctgagtcaaaaataggcagagaggagaaatgttaaaggactccccctgaatctgttagtggttttcaacaggaagattttgactgccagtggtcatttggaaatgtctggagacacttttggttgtcataactagggaagtgggatgctattggcctctagtgggtagaggccaggaatgctgctgaacatcctaccatgcatgagacagcccaaactaaatagtactgaagttaagaaactgctctaaatccaggttgaatggcctgagctcaagcctgccagaaattgagggcagcagtcatccctatgtattctcccctaacaagacccccaagcaagcagtggctctgacttctcccaggccatctcctggaaggctgaggagaactggtggaaatcgaaagcataagcatttttccttccaggtgctggggagtcaggaaagagcaccatcgtcaaacagatgaagtgagtagaaacaaagccccaaaagacaagatagggtgaagaagtcagtacagccagtggaggtattcaaagtgaaaggctctttagcctcaaggagcccaggtataaaggatctgattccaatgcccctcatctgtaccccttgtccctcaccctccactttgagaaagcagtagcaacagagagataggattcttatgatcctttaaatacccccaaattcctaatcccttaggtctggttactcaggtccagctaaagacagagtgtctgccccttgcaggatcattcaccaggatggctattcaccagaagaatgcctggagttcaaggctatcatctatggaaatgtgctgcagtccatcctggctatcatccgggccatgaccacactgggcatcgattatgctgaaccaagctgtgcggtatgtgattactattatgtggttaagggtggaagcagaaaggctagcaagaagaaacataccagaggccaacaaactatatggaaagatgggtaagaaaaatagtaactaaaatcccacctgctgggtgggatctcactgctaggtgtactgtgtataccatctcaaggccctttacccatctacgcagtaagaccttaaggtagacggtacctttctaattagcctcattttacacatggaaaaactgaagttcagagaagttaattaactgcccaaggtcatgtagataaatagcagagactgcatttgcattgggctgcttgactacaaagctgaatatttttcataatacaccacaattatgcagtcctggagaagtaagcaacatccacccttatttttactgggacagggtcttcctctcttacccaggctggaatgcagtggtgtgatcatggctcactgcaatctcaccctcccaggcttaagtgattctcccacctcagccccagtaggtgggaccacaggtgtgcaccaccacacccagctattttttaaaatttttttgtagagaaggggtcttgccatcttgtctaggctggtgtcaaactcctggccttaagcaatcctcctgcctcagactccgaaagtgttgagattacaggtgtgagccaccgtgcctgtcctcaagccctactttataacacatatttacaaaataaagctgctcacagcttcctctgcttttttactttaccatttaaattgctacccttgggttcctggccatggaacttttgtaagtgaaatccctacttctagtactggactttttctagctttcaatcgctgaagaacaaaggtgatgggtccctgtcttctatcttgtctatttcatggtcaatctggcctttacattttcagtctctctgcagcagaggacccacgttttgtggcggtaaactttcagactaaggatggaagccagaaactccagagaggaaatacctgggacccctgccccgctttagacacaccccagggctgggtatagtgccaccctctgcatctggggagcatactcaaaaattcaacagtatgttttcttacatagaatcttcactggatactgcttccatcttaggtcttcgtagtaatgtaaaatgatttccattcagcagtattcccagaagcctcctggaaagctattactcctgtgaagttcttaaccaggtttctgcattacaggatgacgggcgacagctcaacaacctggctgactccattgaggagggaaccatgcctcctgagctcgtggaggtcattaggaggttgtggaaggatggtggggtgcaagcctgcttcgagagagctgcagaataccagcttaatgactccgcatcttagtaagactgactggtgagagggtgggttgatgcttaagcaatcttctagccagtcttctctctggttgggagaaacctcacccaacccaaaatttcaggcattgaaagctggagacccagactgaattcagcctgtggatctgttttgttaggcttcagcaatgttttgaatttaatgctatggggagatctgccacagttgtcatgactttctattgctttacactggctcactacagcctacaaggctgaagatgcaaaattcaaaagacgtgttctgcctagtagccctcagccctgttctcttaattgaggtgtacactaagcctctccttccagaaccctaagatagccctgcagttctgtcatacactctcgcaggagtaatgtgattcatgatggctctttcagggcagaattttctatctggtaatctaggatctacttaggccactaagcagaaatgagtctcctacacctagagcatcttatgtcacgatgccaaggcaggggacacaaactacaagggcaaaccaagggaaggtggtcaggaattagtaccatcattgcaaagctgcttccaaaaagctaagggacataatttatcaatcctacctcagacagtatgggatcatagacaagtatgggcttcaaagtcaaacctgagtttatttaacctaaccatctgtctcctaatcttgtaataatgctatcacttactgttcccggagcactcaaagcccagtcactgctaggtgtactgtgtataccatctcaacgccctttacccatctacgcagtaagaccttaaggtagatggtacctttctaattagcctcattttacacatggaaaaactgaagttcagagaagttaattaactgcccaaggtcatgtagataaatagcagagactgcatttgcattgggctgcttgactacaaagctgaatatttttcataatacaccacaatttctcttgaccctagccaagtatttaactcttctgaatttctccatctgtaaaatggggatatgaaacttgatggcttaggagtttaaacgagaatctgaacattaaaatgcctggcatacaataggcacttgtgttacttgtacttccctcctcctctcagtcatctcagttcacatctgccttcctcttgccttttttttttttggagacggagtctcgctttgtcacccaggctggagtgcagtggcgcaatctcggctcactgccagctttgcctcccgggttcacgccattctcctgcctcagcctcccaagtagctggggatactggcgcccgccaccatgcccagctaattttttgtatttttagtagagatggggtttcaccgtgttagccaggatggtttcgatctcctgaccttgtgatccgcccgcctcggcctcccaaagtactgggattataggcgcgagccaccgtgcctggccaccttttttttttttaaaaaaaataactttttcttttttaaggtaactttcaaacatacaaaagtagaaataacataatgaattaccttcaacagttatcaactcatggccaatcttatttctttttacctccattccctactggattattttgaagcaaattccaggtatcatttcatcctaaaaatgtcattatggatctctaaaatattgaccctttttataaaaagaatataatcacacccaaatagttaacaattccttaatagcatcaaatatcaccaagtcggtgttcaaatttccccagttatctcaaatgtttcctccccagtttgattcaggatataaacaaagtccatgtgttgcattggttcatgtctttaaagtctctttcaatttactgcctcttgatggaacttttagcacccactaaatctcattcaaaaatctacaaaagcttgcagtcagggcaaacccaaggaaaacttatctaacttgatttaaatgcgctggaactccagcctcaaatgggcttgtccctcagtccatcccccatttcctctagttaatgacatatgaaggtttgaaggaaagaatcctaaatttaacaatatggcttctagtcctgatttctatcaccaaattagctgtgtgactttgggtaagataccttttacgtctcttagcctcgtctgtggaaaatttgtcattgatgctaatcacttttctctagctacctgaaccaattagaacgaattacagaccctgagtacctccctagtgagcaagatgtgctccgatccagagtcaaaaccacgggcatcattgaaaccaagttttccgtcaaagacttgaatttcaggtaagtgcatggttccctagggcatctcggatacacgtgtggaatcctgaaaaggggcacaggcaggcttcttggcctttggtgaagcctaatctaaattactgttctgcttctcccccttttcctctcccacttcacggggtgaccatcaaaggcatataggtcgtatgttgggcatacctatgaaaatagctgcttcttccctcaggatgtttgatgtgggagggcagagatccgagagaaagaagtggatccactgcttcgagggagtcacctgcatcattttctgtgcagccctcagtgcctatgatatggtgctggtggaagatgacgaagtggtgagtggcctttgcatcaagcagctttggtagaacaagttctccccatgaccctttctctaagccttgtgtcactctactgccccaacttaggtaatttcagtctagcagccctccagcagaccaatcaatgtctcatgcaaataattctaaaaaacaacttcttctgcaggttctagattagcattttagagctccaaatttactgacagtgagcttggtctcaaattagacatctaagtatcacttggacatcacaaagctcataagaggaattgagtgcaaagagataagggaccatcaactaggcaaagcaaaggagttacacttagtactctcccaaattgcctaaggaaggagatgaaaatgacagaacagagaaaataacatatgatatgaatcttcattgcaacataatagaagggttgagctagtaaccccacttaggaggctaaaaatgtactgtccgtaggagtttaaggagagactggcagaccagctttctctcatgccaattaaattggcagctggaagactacccaagagtggttctctttagcctgtagaattctgtaggacaggagttctataggacaagtgttagagcccagccagtttctgaatttgggaaaggttagaggtgagaaaaacgttaatttcacccaagcatctgctttctgaatttgggaaaagttagaggcgtgaaaaacgttaatttcacccaagcatctgctcacaaatggaggtccacaccttgctgatgacctctgaatttgggaaaggttagaggcgtgaaaaatgttaatttcacccaagcatctgcttacaaatggaggtccacagcttgctgatgaaagggatactcctatcccttgccacagcttgttctcttcccttccctttggtagttttaacttcacattagagcactctgaatatcgtctaatcaaaatgtcttacagagctattcacttcccatctttaagcctaaagattacagtctatgagactttccatctttaaccctaaagtctatgagtctatgaggtttattaaagtctatgagacattaataaaacaagtctatgagaccttaaagggttgtacaggagtattatggggaaaaagccacaaatgggattgttcttgctttattagataagtagactgaacgcaagtcagctgatagtatacttcaaaaccctaaagacctgctccctaaaagcaagctgggctggggcaatgggcagcctctgcagatatgcagcccgacttctcgctaagtagcaatcagagaaggaaatgagagagcagaaatgcttgtggtatggcactgggaaattctctaactctcaccatgtggcagcaggaccaaagtagcccaaactgagatctgggaccccatgaaagaagcctatcaaaatcatcctggagatgcatatgggcacatgctaacttgggcctgtttcaacccattatcagcactacttataaaatgtcaagttctcagttgcatcctggctgctaaagatctgcataacacattatagacctatatgccagccactatcatggacaatatacatacacaatctcatttagctttcattgtaaccctataagataggaaaacagactcagaaaaagctcaataattttccacaagtcacacagctattagaaagatagggaactagaatgatcccacatctctctggctttactctggtacattgagataagctgttctctgtcttgctttttacatttggagcactggtctctccaaggggaacaagagcaggaagtaggtagatattctataagccaaatctgatatttccaatggtgtttcctcttactagaatcgtatgcatgagtctttgcatctgttcaacagcatatgtaaccacaaattctttgcggctacttccattgtcctctttctcaacaagaaggacctctttgaggaaaaaatcaagaaagtccatctcagcatttgttttccagagtatgatggtaagtgtcaggggctggaaataataataatgccttttagtagagactggcaattgtctcattttttaggccaagatgacacaaaggaacttaagggagaaccttgggcacagttacagggtttaaattcagatactctggaatacagcaggcattagatgcaggagagccactgacttcatatgatacctactgaaaaccaaaggtggaaagacacctctcctcaatttcttttcaactaaagtgagaaacactggagtgcaatagagaatcttccctccaaaaataggcccccaactgctgttgtctaataacatttcaaggatcaagtcaatcacctaaagtgagtcagcaactaacaagggttcatttattctactttttcactatttttctggaaaaccaggtaacaactcctatgatgatgcggggaattacataaagagccagttccttgacctcaatatgcgaaaagatgtcaaagaaatctacagtcacatgacctgtgctacagatacacagaatgtcaaatttgtgtttgatgcagttacagatattatcatcaaagaaaacctcaaggactgcggcctcttctaatcctcaccattcctcaggtataagttctataaacaggcttggaatctgggtaattaaaaacagaaaattatagtcaatataccatgacatgaagaatgaatccattctttggagatggagtatacatgactgcaactgtatttcatacgttcttttcaaagtgggatagctattgcagcttaaagagcacaggccagtagttagaagaccccccaggttccagtactggttttccaacttaatacaaaactgtgaatactttatttctgagtcttgagtctttcaactataaaatgaagatgacttccctacctactttacagggttattctgaggagcacgaacataactgaagggaaggcacataaaaactgcttgtgcaggccaggcacagtggctcacgcttgtaatcccagcactttgggaggccaaggtgggcagatcacga

SEQ ID NO: 10 - DNA, 592 bp, Woodchuck hepatitis virus
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 11 - DNA, 542 bp, Woodchuck hepatitis virus
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcg

SEQ ID NO: 12 - DNA, 384 bp, Homo sapiens
ctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagctcctgggcaacgtgctggtctgtgtgctggcccatcactttggc

SEQ ID NO: 13 - DNA, 122 bp, Simian virus 40
aacttgtttattgcagcttataatggttacaaataaagcaatagcatcacaaatttcacaaataaagcatttttttcactgcattctagttgtggtttgtccaaactcatcaatgtatctta

SEQ ID NO: 14 - DNA, 141 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 15 - DNA, 141 bp, Artificial Sequence
  Description of Artificial Sequence Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 16 - DNA, 1727 bp, Homo sapiens
ggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagccatg

SEQ ID NO: 17 - DNA, 2000 bp, Homo sapiens
cccttgaacagtgtagccttcagcatggctctggcctcagtgtgagttaccagtattgtgagtacgctggagcagggctagcagtggggtataattactgaaaccttataaccaagattccaagatagtcacaatttcaaagagactctcctgtttttccttaagtcaataaggacttgccaatttgattgggaaaacatggctaccatggaaagcacctcagcagctgcatatttctccatcagcccctacagtcactgaaaggtggtgcgaaggaggaagataattagctatggctcagggtacctgataggcggggagacctagattctactcctgaccttcccagtcccaactggccactgctgcacaagtccagctctaaaatgaaagagcaaattacatccttcgacctcagtagttctccaccttggctgtacactagaatcagctggggacgtttttaaatccttgtgcctggactgcactccagaccaattaaatcagtattttttaaaaagctctccaggtaaatgccactgtacactaaaagttgagaactgtttcaggccagggtttttcaaacttcacagatgtgataactacctgtattagcttaaaatattttctctggctctggagtttgatgctaacataccatttaaaaacatacactattttaacatctcggaaagcagggtaatcttctagtcagtggcacctaagacttgaggaaacacagacaccagcaacccaggtgattcttaggattaggtaagtttagcggaaaacaggtccctagggcagtacttcccaatctttaatgtctttagagtcacctgagatcttgttaaactgcggattctgaatcagaagttctggattaaagcctgagattctgcatgtctaatgagcttccgggtgatctgatgttgctggctcttgatccacattttgggcaacaaaattctaaaacatctccacctgaggaggctccgccagcaacactcatttaagtaaactgaaataattggcatagaggagtacaacctgtggagtcctaaatgtctgtgaggcagttgctagactgcaccctcatttttcccccaggaaagggcagctgggatgagagccagaaggagagagagctgccccaacctttgagaagccagagtctggagtccaatttcccaaagaagcagagttttttgtgtgaggcagcacaaaccccacactgaataccagcaaagttcatttatgaagtgaagttgggactcagctggctttagtgggccaaagggaagcaaccccattctcttcaccatacacccttttcctgcatttattcattcaccaaaccttttgatccacaaataaactacaagttctgagaggttcaaagatgagtcagatttggtccgttgagctccagggggagaaatgcagtgagggaaaagatttgtaaaacgacgtacaatatgaagtgataagtgctaataacagaagtacaaagagaggggccaatgcaaaggaacaatgctagtagcttcctggagcagcagccactgcgggagctgaaacattcctaatcttcccaaggaagggcaccacccaaaacaaatttcctggccaggaccagcctatggtaaacgagtatgctttgataccctgaagcccttgagatcaagaccttataatctggaggctcaacataaggaatgctttctacatatgtgccagtaatctctagctctatgatgcaaataaatctaaggaagcaagagactttcaggggatgaaccccttaaaggatggaagtagtcgtgcatcctatccttccgtcagaacccagcagatcatttccctagttatagaaacatttgagtctttaccccttgccatattgacaaagctcttaattggcttgacctatcacattgctagatataaaggctacaatccctagactaagaagtaggtctccagttgaagtagggagt

SEQ ID NO: 18 - DNA, 500 bp, Artificial Sequence
  Description of Artificial Sequence Synthetic polynucleotide
catcctgagagatgagccaggacaaagaaccagtaatagctcctggagcagcacatctgttttgccaggattatcccttggatctcttaaaaccgagaccttgtaatctgaagactcaacttgggctgtacccttaaccttcagctctatgatgcaagtgagtccacaggaccggaggctttgagatgagcttttcagaagggaggagttggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagaga

SEQ ID NO: 19 - DNA, 2012 bp, Artificial Sequence
  Description of Artificial Sequence Synthetic polynucleotide
acgcgtagaggcaggccgagtttgaggccagcctggtctacacaggcgttctaggagaacctgtctcatatgcacatgggcctgggattacacatacaactgcaatggcagcacttggaaagcggaagcaggagaatcggaatttcacactcatccttcattatagaagtccaaacctgtgctaagctacttggacatcgctaaaaaacagcaaaaatctttctaggaattgccaatgtatacacaccaagttgtatttttgtagggcagacttttccaacttgccagatgataataatcatttatattagctacatttcaggctccagaattaacactgttactgaaatgtataggttctaaaacataccatttccaaatattttaaaggattaacatttttgaaaagcatgtgatcttccaaccatattttagggaacagacataagaagtaggcaagtttgggaagaaacagttcctgagggcatttcttcccaaccttgaatgccctgtgggttaactgaggtcttggtaaaatgtagattatttactggactttttgaactgagagtgcatttctaacaaaactccagggtatgtggtccttgcattacatatggggtagcaacattctaaagcagtgtttttcaacctgtggatcaagacccacagaagtcacatagcagatatcctgcctattagatatttacattaagattcagagcagtagcagaataggagtcatcccaacctgaggaactgcatcagagtcccagcatcaggaagggtgagagccactgagctaaagtcctctaggtgagggggctgccccggaaagactcatttaaatgaaaccactgacacagagagctgacagatgaggtgggttccgtgtctgtgaggctctgctggtcgtctcctcactcccatggaagaaccaccgagatgagggcgaggggcagagctaacccagcctctaagtaggcagagtctagtgtccagctgcccaaggaagaagtttgctgtgtgaggtggccctgatgtccgcacacacaaaatgccagtgaagtctacttgaccaagtgaagctggtgtggaatgggaagaagcacacacagtcagtctctctgcacacactctgtccttcacttcttcacttaccagagatttgatgagaacctactagcagatcagatttgatccctgagtggaaaaaacgtacagtgggagataaaagaggaaaacaacggatctgagtttgaggttagcctagtctgagcaagccggatacacagtaagaccctgtctcacatacatccactcgcacgcgcgcgcacacaaacacacacacacacacacacacacacacacacacacacacacatcgtgctaaggacaagataggcatcctgagagatgagccaggacaaagaaccagtaatagctcctggagcagcacatctgttttgccaggattatcccttggatctcttaaaaccgagaccttgtaatctgaagactcaacttgggctgtacccttaaccttcagctctatgatgcaagtgagtccacaggaccggaggctttgagatgagcttttcagaagggaggagttggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagaatgttactctatcctaacatatttttccttttcctctatctcacagataggaaaaatttaagagccagagggaacgtcccttctcagaggagacagcagaaggatcc

SEQ ID NO: 20 - DNA, 133 bp, Simian virus 40
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 21 - DNA, 66 bp, Artificial Sequence
  Description of Artificial Sequence Synthetic oligonucleotide
ggaagcggagctactaacttcagcctgctgaagcaggctggagacgtggaggagaaccctggacct

SEQ ID NO: 22 - DNA, 66 bp, Artificial Sequence
  Description of Artificial Sequence Synthetic oligonucleotide
ggcagcggcgccacaaacttctctctgctaaagcaagcaggtgatgttgaagaaaaccccgggcct

SEQ ID NO: 23 - DNA, 63 bp, Artificial Sequence
  Description of Artificial Sequence Synthetic oligonucleotide
ggctccggcgagggcaggggaagtcttctaacatgcggggacgtggaggaaaatcccggccca

SEQ ID NO: 24 - DNA, 69 bp, Artificial Sequence
  Description of Artificial Sequence Synthetic oligonucleotide
ggctcgggccagtgtactaattatgctctcttgaaattggctggagatgttgagagcaacccaggtccc

SEQ ID NO: 25 - DNA, 479 bp, Bos taurus
acgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtcctt

SEQ ID NO: 26 - DNA, 7265 bp, Artificial Sequence
  Description of Artificial Sequence Synthetic polynucleotide
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacagtttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcctacagcagccagggtgagattatgaggctgagctgagaatatcaagactgtaccgagtagggggccttggcaagtgtggagagcccggcagctggggcagagggcggagtacggtgtgcgtttacggacctcttcaaacgaggtaggaaggtcagaagtcaaaaagggaacaaatgatgtttaaccacacaaaaatgaaaatccaatggttggatatccattccaaatacacaaaggcaacggataagtgatccgggccaggcacagaaggccatgcacccgtaggattgcactcagagctcccaaatgcataggaatagaagggtgggtgcaggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagcggtaccatggtgatgttcaagaagatcaagtcttttgaggtggtcttcaacgaccccgagaaagtgtacggcagcggggagaaggtggccggacgggtaattgtggaagtgtgtgaagttacccgagtcaaagctgtcaggatcctggcttgcggcgtggccaaggtcctgtggatgcaagggtctcagcagtgcaaacagactttggactacttgcgctatgaagacacacttctcctagaagagcagcctacagcaggtgagaacgagatggtgatcatgaggcctggaaacaaatatgagtacaagttcggcttcgagcttcctcaagggcccctgggaacatcctttaaaggaaaatatggttgcgtagactactgggtgaaggcttttctcgatcgccccagccagccaactcaagaggcaaagaaaaacttcgaagtgatggatctagtggatgtcaatacccctgacttaatggcaccagtgtctgccaaaaaggagaagaaagtttcctgcatgttcattcctgatggacgtgtgtcagtctctgctcgaattgaccgaaaaggattctgtgaaggtgatgacatctccatccatgctgactttgagaacacgtgttcccgaatcgtggtccccaaagcggctattgtggcccgacacacttaccttgccaatggccagaccaaagtgttcactcagaagctgtcctcggtcagaggcaatcacattatctcagggacttgcgcatcgtggcgtggcaagagcctcagagtgcagaagatcagaccatccatcctgggctgcaacatcctcaaagtcgaatactccttgctgatctacgtcagtgtccctggctccaagaaagtcatccttgatctgcccctagtgattggcagcaggtctggtctgagcagccggacatccagcatggccagccggacgagctctgagatgagctggatagacctaaacatcccagataccccagaagctcctccttgctatatggacatcattcctgaagatcacagactagagagccccaccacccctctgctggacgatgtggacgactctcaagacagccctatctttatgtacgcccctgagttccagttcatgcccccacccacttacactgaggtggatccgtgcgtccttaacaacaacaacaacaacaacaacgtgcagtagtggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 27 - protein, 90 aa, Artificial Sequence [sequence omitted]

SEQ ID NO: 28 - protein, 99 aa, Artificial Sequence [sequence omitted]

SEQ ID NO: 29 - protein, 397 aa, Artificial Sequence
  Description of Artificial Sequence Synthetic polypeptide
MVMFKKIKSFEVVFNDPEKVYGSGEKVAGRVIVEVCEVTRVKAVRILACGVAKVLWMQGSQQCKQTLDYLRYEDTLLLEEQPTAGENEMVIMRPGNKYEYKFGFELPQGPLGTSFKGKYGCVDYWVKAFLDRPSQPTQEAKKNFEVMDLVDVNTPDLMAPVSAKKEKKVSCMFIPDGRVSVSARIDRKGFCEGDDISIHADFENTCSRIVVPKAAIVARHTYLANGQTKVFTQKLSSVRGNHIISGTCASWRGKSLRVQKIRPSILGCNILKVEYSLLIYVSVPGSKKVILDLPLVIGSRSGLSSRTSSMASRTSSEMSWIDLNIPDTPEAPPCYMDIIPEDHRLESPTTPLLDDVDDSQDSPIFMYAPEFQFMPPPTYTEVDPCVLNNNNNNNNVQ

SEQ ID NO: 30 - protein, 102 aa, Artificial Sequence
  Description of Artificial Sequence Synthetic polypeptide
MDVCQSLLELTEKDSVKVMTSPSMLTLRTRVPESWSPKRLLWPDTLTLPMARPKCSLRSCPRSEAITLSQGLAHRGVARASECRRSDHPSWAATSSKSNTPC

SEQ ID NO: 31 - protein, 249 aa, Artificial Sequence
  Description of Artificial Sequence Synthetic polypeptide
MWTTLKTALSLCTPLSSSSCPHPLTLRWIRASLTTTTTTTTCSSGSNQPLDYKICERLTGILNYVAPFTLCGYAALMPLYHAIASRMAFIFSSLYKSWLLSLYEELWPVVRQRGVVCTVFADATPTGWGIATTCQLLSGTFAFPLPIATAELIAACLARCWTGARLLGTDNSVVLSGKLTSFPWLLACVATWILRGTSFCYVPSALNPADLPSRGLLPALRPLPRLRDLPRLCLLVASHLLFAPPPCLP

SEQ ID NO: 32 - protein, 98 aa, Artificial Sequence
  Description of Artificial Sequence Synthetic polypeptide
MLLLPVWLSFSPPCINPGCCLFMRSCGPLSGNVAWCALCLLTQPPLVGALPPPVSSFPGLSLSPSLLPRRNSSPPALPAAGQGLGCWALTIPWCCRGS

SEQ ID NO: 33 - protein, 78 aa, Artificial Sequence
  Description of Artificial Sequence Synthetic polypeptide
MAARLCCHLDSARDVLLLRPFGPQSSGPSFPRPAAGSAASSASSRSASTVPSSCQPSVVCPSPVPSLTLEGATPTVLS

SEQ ID NO: 34 - protein, 97 aa, Artificial Sequence
  Description of Artificial Sequence Synthetic polypeptide
MPAIVFPILPLAVLPHPTPQNRMTPTQTMRCNFLILLGKDSGSGTFQGQGRHGGGANNRWLATRRHSRGRSRRRGRGRRAGSRPREGRSAGLRAEGT

SEQ ID NO: 35 - protein, 286 aa, Artificial Sequence
  Description of Artificial Sequence Synthetic polypeptide
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVVMVTTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 36 - protein, 88 aa, Artificial Sequence
  Description of Artificial Sequence Synthetic polypeptide
MIPRDPRSPAPDLSAINQPAGRAERRSGPATLSASIQSINCCREARVSSSPVNSLRNVVTITTGIVVSRSSFGMASFSSGSQRSRRVT

SEQ ID NO: 37 - DNA, 43 bp, Artificial Sequence
  Description of Artificial Sequence Synthetic primer
acagggctttccatagcggtaccatggtgatgttcaagaagat

SEQ ID NO: 38 - DNA, 40 bp, Artificial Sequence
  Description of Artificial Sequence Synthetic primer
ccagaggttgattggatccactactgcacgttgttgttgt

SEQ ID NO: 39 - DNA, 5038 bp, Artificial Sequence
  Description of Artificial Sequence Synthetic polynucleotide
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgcacgcgtcatcctgagagatgagccaggacaaagaaccagtaatagctcctggagcagcacatctgttttgccaggattatcccttggatctcttaaaaccgagaccttgtaatctgaagactcaacttgggctgtacccttaaccttcagctctatgatgcaagtgagtccacaggaccggaggctttgagatgagcttttcagaagggaggagttggccgcttgctcccagagctccagcacctgcattcttctggctatgtcagaagccagatcatttccctcgttaaaaacaaaaacaaaaaaacaaacaaacaaaatgttagtctttgccctttatctgcctggcaaagcttttaattggcttgatctgtcattccgctagacataaaggggacaatccccggattaggaaggagctctccagctcgggtaaggagtctcaaggcaaggtaggcaagcaccaccggtccgcactctcgcccagcttttacgggaagaagagagggtaccatggtgatgttcaagaagatcaagtcttttgaggtggtcttcaacgaccccgagaaagtgtacggcagcggggagaaggtggccggacgggtaattgtggaagtgtgtgaagttacccgagtcaaagctgtcaggatcctggcttgcggcgtggccaaggtcctgtggatgcaagggtctcagcagtgcaaacagactttggactacttgcgctatgaagacacacttctcctagaagagcagcctacagcaggtgagaacgagatggtgatcatgaggcctggaaacaaatatgagtacaagttcggcttcgagcttcctcaagggcccctgggaacatcctttaaaggaaaatatggttgcgtagactactgggtgaaggcttttctcgatcgccccagccagccaactcaagaggcaaagaaaaacttcgaagtgatggatctagtggatgtcaatacccctgacttaatggcaccagtgtctgccaaaaaggagaagaaagtttcctgcatgttcattcctgatggacgtgtgtcagtctctgctcgaattgaccgaaaaggattctgtgaaggtgatgacatctccatccatgctgactttgagaacacgtgttcccgaatcgtggtccccaaagcggctattgtggcccgacacacttaccttgccaatggccagaccaaagtgttcactcagaagctgtcctcggtcagaggcaatcacattatctcagggacttgcgcatcgtggcgtggcaagagcctcagagtgcagaagatcagaccatccatcctgggctgcaacatcctcaaagtcgaatactccttgctgatctacgtcagtgtccctggctccaagaaagtcatccttgatctgcccctagtgattggcagcaggtctggtctgagcagccggacatccagcatggccagccggacgagctctgagatgagctggatagacctaaacatcccagataccccagaagctcctccttgctatatggacatcattcctgaagatcacagactagagagccccaccacccctctgctggacgatgtggacgactctcaagacagccctatctttatgtacgcccctgagttccagttcatgcccccacccacttacactgaggtggatccgtgcgtccttaacaacaacaacaacaacaacaacgtgcagtagccgctcgagataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttagttcttgccacggcggaactcatcgccgcctgccttgcccgctgctggacaggggctcggctgttgggcactgacaattccgtggtgtttatttgtgaaatttgtgatgctattgctttatttgtaaccatctagctttatttgtgaaatttgtgatgctattgctttatttgtaaccattataagctgcaataaacaagttaacaacaacaattgcattcattttatgtttcaggttcagggggagatgtgggaggttttttaaagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 40 - protein, 104 aa, Artificial Sequence
  Description of Artificial Sequence Synthetic polypeptide
MQVLELWEQAANSSLLKSSSQSLRSCGLTCIIELKVKGTAQVESSDYKVSVLRDPRDNPGKTDVLLQELLLVLCPGSSLRMTRAAAGTPSDGVGHSLSARSLAH

SEQ ID NO: 41 - protein, 81 aa, Artificial Sequence
  Description of Artificial Sequence Synthetic polypeptide
MRCEIPHRCVRRKYRIRRPCRQLRARSLTEAARAKPGRRATFGRPASVSERARREGVANSITRGSCGRFKKPPTSPPEPET

SEQ ID NO: 42 - protein, 74 aa, Artificial Sequence
  Description of Artificial Sequence Synthetic polypeptide
MELATPSLRARSLTEAGRPKVARRPGFARAASVSERARSCLQGRLMRYFLLTHLCGISHRIRQSNHSTRPVAAH

SEQ ID NO: 43 - protein, 286 aa, Artificial Sequence
  Description of Artificial Sequence Synthetic polypeptide
MSIQHFRVALIPFFAAFCLPVFAHPETLVKVKDAEDQLGARVGYIELDLNSGKILESFRPEERFPMMSTFKVLLCGAVLSRIDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPVAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 44 - protein, 88 aa, Artificial Sequence
  Description of Artificial Sequence Synthetic polypeptide
MIPRDPRSPAPDLSAINQPAGRAERRSGPATLSASIQSINCCREARVSSSPVNSLRNVVAIATGIVVSRSSFGMASFSSGSQRSRRVT

SEQ ID NO: 45 - DNA, 20 bp, Artificial Sequence
  Description of Artificial Sequence Synthetic primer
ctcgggtaaggagtctcaag

SEQ ID NO: 46 - DNA, 40 bp, Artificial Sequence
  Description of Artificial Sequence Synthetic primer
cttttacgggaagaagagagggtaccatggtgatgttcaa

SEQ ID NO: 47 - DNA, 40 bp, Artificial Sequence
  Description of Artificial Sequence Synthetic primer
aggttgattatctcgagcggctactgcacgttgttgttgt

SEQ ID NO: 48 - DNA, 20 bp, Artificial Sequence
  Description of Artificial Sequence Synthetic primer
aagcagcgtatccacatagc

SEQ ID NO: 49 - DNA, 7072 bp, Artificial Sequence
  Description of Artificial Sequence Synthetic polynucleotide
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagcctacagcagccagggtgagattatgaggctgagctgagaatatcaagactgtaccgagtagggggccttggcaagtgtggagagcccggcagctggggcagagggcggagtacggtgtgcgtttacggacctcttcaaacgaggtaggaaggtcagaagtcaaaaagggaacaaatgatgtttaaccacacaaaaatgaaaatccaatggttggatatccattccaaatacacaaaggcaacggataagtgatccgggccaggcacagaaggccatgcacccgtaggattgcactcagagctcccaaatgcataggaatagaagggtgggtgcaggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagcatatggcaacccttaaggagaagctcattgcgtccgttgcagatgatgaggctgccgtcccgaacaacaagatcactgtagtgggcgttggacaagtgggtatggcatgtgccatcagcattctgggaaagtctctggctgatgaacttgccctggtggatgtgttggaagacaagctcaaaggagagatgatggacctgcagcacgggagcttgttcctccagactccgaaaattgtggccgataaagattactctgtgacagccaactctaagattgtggtggtgacggcaggagtccgccagcaggagggggagagtcggctcaacctggtgcagagaaatgtcaacgtgttcaagttcatcattcctcagatcgtcaagtacagccctgactgcaccatcatcgtggtttccaacccagtggatattctgacttacgtcacctggaaactgagcgggctacctaagcaccgtgtgattggaagcggatgcaatctggattctgctcgattccgctacctcatggcagagaagcttggcattcatcccagcagctgccacggatggatcctgggcgagcatggagactccagtgtggctgtgtggagcggggtgaatgtggcaggagtctccctccaggaactgaatccagaaatggggacagacaatgacagtgagaactggaaggaggtgcataagatggtggtggacagtgcctatgaagtcatcaagctcaaaggctacaccaactgggccatcggcctgagcgtggctgacctcatcgagtccatgctgaaaaacctctcccggattcaccccgtgtctaccatggtgaagggaatgtacggcattgagaatgaagtcttcctcagtctcccgtgcatcctcaatgctcgggggctgaccagcgtcatcaatcagaagctgaaggacgatgaggtcgctcagctcaggaagagtgcggacaccctgtgggacatccagaaagacctcaaagacctgtgatggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 50 - protein, 86 aa, Artificial Sequence
  Description of Artificial Sequence Synthetic polypeptide
MLMAHAIPTCPTPTTVILLFGTAASSSATDAMSFSLRVAICYGKPCPRRQPALAHHLRVTALYTGLWDQPGPLHLKSSQTLHYLIS

SEQ ID NO: 51 - protein, 334 aa, Artificial Sequence
  Description of Artificial Sequence Synthetic polypeptide
MATLKEKLIASVADDEAAVPNNKITVVGVGQVGMACAISILGKSLADELALVDVLEDKLKGEMMDLQHGSLFLQTPKIVADKDYSVTANSKIVVVTAGVRQQEGESRLNLVQRNVNVFKFIIPQIVKYSPDCTIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVDSAYEVIKLKGYTNWAIGLSVADLIESMLKNLSRIHPVSTMVKGMYGIENEVFLSLPCILNARGLTSVINQKLKDDEVAQLRKSADTLWDIQKDLKDL

SEQ ID NO: 52 - protein, 78 aa, Artificial Sequence
  Description of Artificial Sequence Synthetic polypeptide
MHLLPVLTVIVCPHFWIQFLEGDSCHIHPAPHSHTGVSMLAQDPSVAAAGMNAKLLCHEVAESSRIQIASASNHTVLR

SEQ ID NO: 53 - protein, 229 aa, Artificial Sequence
  Description of Artificial Sequence Synthetic polypeptide
MRSLSSGRVRTPCGTSRKTSKTCDGSNQPLDYKICERLTGILNYVAPFTLCGYAALMPLYHAIASRMAFIFSSLYKSWLLSLYEELWPVVRQRGVVCTVFADATPTGWGIATTCQLLSGTFAFPLPIATAELIAACLARCWTGARLLGTDNSVVLSGKLTSFPWLLACVATWILRGTSFCYVPSALNPADLPSRGLLPALRPLPRLRDLPRLCLLVASHLLFAPPPCLP

SEQ ID NO: 54 - DNA, 19 bp, Artificial Sequence
  Description of Artificial Sequence Synthetic primer
agtataaagcgccgtgacc

SEQ ID NO: 55 - DNA, 42 bp, Artificial Sequence
  Description of Artificial Sequence Synthetic primer
gggacagggctttccatagcatatggcaacccttaaggagaa

SEQ ID NO: 56 - DNA, 20 bp, Artificial Sequence
  Description of Artificial Sequence Synthetic primer
acaagtgggtatggcatgtg

SEQ ID NO: 57 - DNA, 21 bp, Artificial Sequence
  Description of Artificial Sequence Synthetic primer
acaattttcggagtctggagg

SEQ ID NO: 58 - DNA, 19 bp, Artificial Sequence
  Description of Artificial Sequence Synthetic primer
agcgacctcatcgtccttc

SEQ ID NO: 59 - DNA, 40 bp, Artificial Sequence
  Description of Artificial Sequence Synthetic primer
ccagaggttgattggatccatcacaggtctttgaggtctt

(19) United States
(12) Patent Application Publication
(10) Pub. No.: US 2022/0118107 A1
(43) Pub. Date: Apr. 21, 2022

(54) TXNIP AND LDHB COMPOSITIONS AND METHODS FOR THE TREATMENT OF DEGENERATIVE OCULAR DISEASES

(71) Applicant: President and Fellows of Harvard College, Cambridge, MA (US)

(72) Inventors: Yunlu Xue, Boston, MA (US); Constance L. Cepko, Newton, MA (US)

(21) Appl. No.: 17/428,670

(22) PCT Filed: Feb. 11, 2020
(86) PCT No.: PCT/US2020/017669

§ 371 Date: Aug. 5, 2021

Related U.S. Application Data
(60) Provisional application No. 62/803,680, filed on Feb. 11, 2019.

(51) Int. Cl. A61K 48/00; A61P 27/02; A61P 39/06; A61K 38/17; C12N 15/86

(52) U.S. Cl. CPC A61K 48/005; A61P 27/02; A61P 39/06; C07K 14/47; A61K 48/0075; C12N 15/86; A61K 38/17

(57) ABSTRACT

The present invention provides compositions, e.g., pharmaceutical compositions, which include a recombinant adeno-associated viral (AAV) expression construct, AAV vectors, AAV particles, and methods of treating a subject having a degenerative ocular disorder, e.g., retinitis pigmentosa.

### RELATED APPLICATIONS

The present application claims the benefit of priority to U.S. Provisional Application No. 62/803,680, filed on Feb. 11, 2019, the entire contents of which are incorporated herein by reference.

### GOVERNMENT FUNDING

This invention was made with Government support under contract numbers U01 EY025497 and EY023291-03 awarded by the National Eye Institute (NEI). The government has certain rights in the invention.

### SEQUENCE LISTING

The instant application contains a Sequence Listing which has been submitted electronically in ASCII format and is hereby incorporated by reference in its entirety. Said ASCII copy, created on Feb. 11, 2020, is named 117823_19320_SL.txt and is 142,548 bytes in size.

### BACKGROUND OF THE INVENTION

Retinitis pigmentosa (RP) is a disease of the eye that presents with progressive degeneration of rod and cone photoreceptors, the light-sensing cells of the retina (Hartong D T, et al. (2006) Lancet 368(9549):1795-1809). The disease can result from mutations in any of over 60 different genes and is the most common inherited form of blindness in the world, affecting an estimated 1 in 4000 individuals (Daiger S P, et al. (2013) Clin Genet 84(2):132-141; Berson E L (1996) Proc Natl Acad Sci USA 93(10):4526-8; Haim M (2002) Acta Ophthalmol Scand Suppl (233):1-34). One approach to treat this disease is gene therapy, e.g. using adeno-associated vectors (AAVs) to deliver a wild-type allele to complement a mutated gene (Ali R R, et al. (1996) Hum Mol Genet 5(5):591-4; Murata T, et al. (1997) Ophthalmic Res 29(5):242-251). While this approach has proven successful in other conditions, even leading to the approval of a gene therapy for RPE65-associated Leber's congenital amaurosis (Maguire A M, et al. (2008) N Engl J Med 358(21):2240-2248), it is difficult to implement for the majority of RP patients, given the extensive heterogeneity of genetic lesions (Daiger S P, et al. (2013) Clin Genet 84(2):132-141). A broadly applicable gene therapy that is agnostic to the genetic lesion would provide a treatment option for a greater number of RP patients. Presently, there is no effective therapy of any kind for RP, and despite more than a dozen randomized clinical trials to date, none have been able to demonstrate an improvement in visual function (Sacchetti M, et al. 2015) J Ophthalmol 2015:737053).

In patients with RP, there is an initial loss of rods, the photoreceptors that mediate vision in dim light. Clinically, this results in the first manifestation of RP, poor or no night vision, which usually occurs between birth and adolescence (Hartong D T, et al. (2006) Lancet 368(9549):1795-1809). Daylight vision in RP is largely normal for decades, but eventually deteriorates beginning when most of the rods have died. This is due to dysfunction, and then death, of the cone photoreceptors, which are essential for high acuity and color vision. Loss of cone function is the major source of morbidity in the disease (Hartong D T, et al. (2006) Lancet 368(9549):1795-1809). Importantly, while the vast majority of genes implicated in RP are expressed in rods, few actually exhibit expression in cones, suggesting the existence of one or more common mechanisms by which diverse mutations in rods trigger non-autonomous cone degeneration (Narayan D S, et al. (2016) Acta Ophthalmol 94(8):748-754; Wang W, et al. (2016) Cell Rep 15(2):372-85; Komeima K, et al. (2006) Proc Natl Acad Sci USA 103(30):11300-5). Attempts to elucidate these mechanisms have been made with the goal of developing therapies for RP that preserve cone vision regardless of the underlying mutation (Punzo C, et al. (2009) Nat Neurosci 12(1):44-52; Xiong W, et al. (2015) J Clin Invest 125(4):1433-1445; Venkatesh A, et al. (2015) J Clin Invest 125(4):1446-58; Aït-Ali N, et al. (2015) Cell 161(4):817-832; Murakami Y, et al. (2012) Proc Natl Acad Sci 109(36):14598-14603).

Accordingly, there is a need in the art for therapies to treat and prevent vision loss that results from degenerative retinal diseases, such as RP.

### SUMMARY OF THE INVENTION

The present invention is based, at least in part on the discovery of mutation-independent compositions and methods of treatment for subjects having RP.

More specifically, it has surprisingly been discovered that intraocular delivery of AAV comprising thioredoxin interacting protein (TXNIP) prolongs survival of cones in RP-mutant mice. Even more surprising, this TXNIP-mediated effect was only observed when TXNIP was specifically expressed in cones. It has also surprisingly been discovered that overexpression of TXNIP causes up-regulation of lactate dehydrogenase B (LDHB) in RP cones and, further, that LDHB expression is necessary for the TXNIP-mediated rescue of cones.

Accordingly, the present invention provides compositions, e.g., pharmaceutical compositions, which include a recombinant adeno-associated virus (AAV) vector, and methods of treating a subject having a degenerative ocular disorder, e.g., retinitis pigmentosa.

In one aspect, the present invention provides a composition, comprising an adeno-associated virus (AAV) expression cassette, comprising a photoreceptor-specific (PR-specific) promoter and a nucleic acid molecule encoding thioredoxin-interacting protein (TXNIP).

In another aspect, the present invention provides a composition, comprising an adeno-associated virus (AAV) expression cassette, the expression cassette comprising a photoreceptor-specific (PR-specific) promoter and a nucleic acid molecule encoding lactate dehydrogenase B (LDHB).

In one aspect, the present invention provides a composition, comprising an adeno-associated virus (AAV) expression cassette, the expression cassette comprising a photoreceptor-specific (PR-specific) promoter and a nucleic acid molecule encoding thioredoxin-interacting protein (TXNIP) and a nucleic acid molecule encoding lactate dehydrogenase B (LDHB).

In another aspect, the present invention provides a composition, comprising a first adeno-associated virus (AAV) expression cassette, the expression cassette comprising a photoreceptor-specific (PR-specific) promoter and a nucleic acid molecule encoding thioredoxin-interacting protein (TXNIP), and a second adeno-associated virus (AAV) expression cassette, the expression cassette comprising a photoreceptor-specific (PR-specific) promoter and a nucleic acid molecule encoding lactate dehydrogenase B (LDHB).

In one embodiment, the PR-specific promoter is a human red opsin (hRedO) promoter.

In one embodiment, the hRedO promoter comprises nucleotides 452-2017 of SEQ ID NO:8 directly linked, i.e., containing no intervening sequences, to nucleotides 4541-5032 of SEQ ID NO:12; or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 452-2017 of SEQ ID NO:8 directly linked to nucleotides 4541-5032 of SEQ ID NO:8.

In another embodiment, the hRedO promoter comprises the nucleotide sequence of SEQ ID NO:16, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of SEQ ID NO:16.

In one embodiment, the hRedO promoter comprises nucleotides 457-2514 of the nucleotide sequence depicted in FIG. 11 (SEQ ID NO:26), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 457-2514 of the nucleotide sequence depicted in FIG. 11 (SEQ ID NO:26).

In another embodiment, the hRedO promoter comprises nucleotides 457-2514 of the nucleotide sequence depicted in FIG. 15 (SEQ ID NO: 49), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 457-2514 of the nucleotide sequence depicted in FIG. 15 (SEQ ID NO: 49).

In one embodiment, the PR-specific promoter is a human guanine nucleotide-binding protein G subunit alpha-2 (GNAT2) promoter.

In one embodiment, the GNAT 2 promoter comprises nucleotides 4873-6872 of SEQ ID NO:9; or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 4873-6872 of SEQ ID NO:9.

In another embodiment, the GNAT 2 promoter comprises the nucleotide sequence of SEQ ID NO:17; or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of the nucleotide sequence of SEQ ID NO:17.

In one embodiment, the GNAT 2 promoter comprises the nucleotide sequence of SEQ ID NO:18; or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of the nucleotide sequence of SEQ ID NO:18.

In another embodiment, the GNAT 2 promoter comprises the nucleotide sequence of SEQ ID NO:19; or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of the nucleotide sequence of SEQ ID NO:19.

In yet another embodiment, the GNAT 2 promoter comprises nucleotides 156-655 of the nucleotide sequence depicted in FIG. 13 (SEQ ID NO: 39), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 156-655 of the nucleotide sequence depicted in FIG. 13 (SEQ ID NO: 39).

In one embodiment, the nucleic acid molecule encoding TXNIP comprises nucleotides 366-1541 of SEQ ID NO:1; or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 366-1541 of SEQ ID NO:1.

In another embodiment, the nucleic acid molecule encoding TXNIP comprises nucleotides 162-1172 of SEQ ID NO:2, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 162-1172 of SEQ ID NO:2.

In yet another embodiment, the nucleic acid molecule encoding TXNIP comprises nucleotides 280-1473 of SEQ ID NO:3; or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 280-1473 of SEQ ID NO:3.

In one embodiment, the nucleic acid molecule encoding TXNIP comprises nucleotides 280-1470 of SEQ ID NO:4, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 280-1470 of SEQ ID NO:4.

In another embodiment, the nucleic acid molecule encoding TXNIP comprises nucleotides 2521-3714 of the nucleotide sequence depicted in FIG. 11 (SEQ ID NO:26), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 2521-3714 of the nucleotide sequence depicted in FIG. 11 (SEQ ID NO:26).

In another embodiment, the nucleic acid molecule encoding TXNIP comprises nucleotides 663-1856 of the nucleotide sequence depicted in FIG. 13 (SEQ ID NO: 39), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 663-1856 of the nucleotide sequence depicted in FIG. 13 (SEQ ID NO: 39).

In one embodiment, the nucleic acid molecule encoding LDHB comprises nucleotides 112-1116 of SEQ ID NO:5; or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 112-1116 of SEQ ID NO:5.

In another embodiment, the nucleic acid molecule encoding LDHB comprises nucleotides 334-1338 of SEQ ID NO:6, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 334-1338 of SEQ ID NO:6.

In another embodiment, the nucleic acid molecule encoding LDHB comprises nucleotides 112-1116 of SEQ ID NO:7, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 112-1116 of SEQ ID NO:7.

In yet another embodiment, the nucleic acid molecule encoding LDHB comprises nucleotides 2517-3521 of the nucleotide sequence depicted in FIG. 15 (SEQ ID NO: 49), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 2517-3521 of the nucleotide sequence depicted in FIG. 15 (SEQ ID NO: 49).

In one embodiment, the expression cassette further comprises a linker, such a a 2A linker.

In one embodiment, the expression cassette further comprises an intron.

In one embodiment, the expression cassette further comprises a post-transcriptional regulatory region.

In another embodiment, the expression cassette further comprises a Woodchuck hepatitis virus posttranscriptional regulatory element (WPRE).

In one embodiment, the expression cassette further comprises a polyadenylation signal.

In one embodiment, the polyadenylation signal is a bovine growth hormone polyadenylation signal or an SV40 polyadenylation signal.

In one embodiment, the expression cassette is present in a vector.

In one embodiment, the vector is an AAV vector selected from the group consisting of AAV2, AAV 8, AAV2/5, and AAV 2/8.

The present invention also provides AAV vector particles and pharmaceutical compositions comprising the AAV compositions of the invention and isolated cells comprising the AAV particles of the invention.

In one embodiment, the pharmaceutical compositions of the invention further comprise a viscosity inducing agent.

In one embodiment, the pharmaceutical compositions of the invention are for intraocular administration.

In one embodiment, the intraocular administration is selected from the group consisting of intravitreal or subretinal, subvitreal, subconjuctival, sub-tenon, periocular, retrobulbar, suprachoroidal, and/or intrascleral administration.

In one aspect, the present invention provides a method for prolonging the viability of a photoreceptor cell compromised by a degenerative ocular disorder. The method includes contacting the cell with any one or more of the AAV composition or the pharmaceutical composition of the invention, or the AAV viral particle of the invention, thereby prolonging the viability of the photoreceptor cell compromised by the degenerative ocular disorder.

In another aspect, the present invention provides a method for treating or preventing a degenerative ocular disorder in a subject. The methods includes administering to the subject a therapeutically effective amount of any one or more of the AAV composition or the pharmaceutical composition of the invention, or the AAV viral particle of the invention, thereby treating or preventing said degenerative ocular disorder.

In another aspect, the present invention provides a method for delaying loss of functional vision in a subject having a degenerative ocular disorder. The methods includes administering to the subject a therapeutically effective amount of any one or more of the AAV composition or the pharmaceutical composition of the invention, or the AAV viral particle of the invention, thereby treating or preventing said degenerative ocular disorder.

In one embodiment, the degenerative ocular disorder is associated with decreased viability of cone cells and/or decreased viability of rod cells.

In one embodiment, the degenerative ocular disorder is selected from the group consisting of retinitis pigmentosa, age related macular degeneration, cone rod dystrophy, and rod cone dystrophy.

In one embodiment, the degenerative ocular disorder is a genetic disorder.

In one embodiment, the degenerative ocular disorder is not associated with blood vessel leakage and/or growth.

In one embodiment, the degenerative ocular disorder is retinitis pigmentosa.

In one aspect, the present invention provides a method for treating or preventing retinitis pigmentosa in a subject. The methods includes administering to the subject a therapeutically effective amount of the composition of any one or more of the AAV composition or the pharmaceutical composition of the invention, or the AAV viral particle of the invention, thereby treating or preventing retinitis pigmentosa in said subject.

Other features and advantages of the invention will be apparent from the following detailed description and claims.

### BRIEF DESCRIPTION OF THE DRAWINGS

FIGS. 1A-1B are fluorescent microscopic images of contralateral retinas from an rd1 homozygous mouse showing an increase of cone cells resulting from AAV-mediated expression of TXNIP driven by a human RedO promoter (FIG. 1B), compared to AAV-mediated expression of H2BGFP driven by a human RedO promoter as a control (FIG. 1A).

FIG. 2A is a scatter-plot distribution of ½ radius cone cell counts of retinas from rd1 homozygous mice without and with AAV-mediated expression of TXNIP driven by a human RedO promoter.

FIG. 2B are schematics exemplary expression constructs used in FIG. 2A. The top exemplary expression construct comprises a photoreceptor-specific promoter, human red opsin (RedO) operably linked to a nucleotic acid molecule encoding thioredoxin-interacting protein (TXNIP), and further comprising a Woodchuck hepatitis virus post-transcriptional regulatory element (WPRE) and a bovine growth hormone polyadenylation signal (BGH pA). The bottom exemplary construct comprises a RedO promoter operably linked to a nucleic acid molecule encoding green fluorescent protein for use as a control (bottom). These constructs were used in Figu

FIG. 3A is a scatter-plot distribution of ½ radius cone cell counts of retinas from rd1 homozygous mice with and without AAV-mediated expression of TXNIP driven by a human RedO promoter or by a guanine nucleotide-binding protein G subunit alpha-2 (GNAT2) promoter (SynPV1).

FIG. 3B is a schematic of an exemplary expression construct comprising a photoreceptor-specific promoter guanine nucleotide-binding protein G subunit alpha-2 (GNAT2) promoter (SynPV1) operably linked to a nucleotic acid molecule encoding thioredoxin-interacting protein (TXNIP), and further comprising a Woodchuck hepatitis virus post-transcriptional regulatory element (WPRE) and an SV40 polyadenylation signal, used in FIG. 3A.

FIG. 4A is a scatter-plot distribution of ½ radius cone cell counts of retinas from rd1 homozygous mice with and without AAV-mediated expression of TXNIP driven by a guanine nucleotide-binding protein G subunit alpha-2 (GNAT2) promoter (SynP136).

FIG. 4B is a schematic of an exemplary expression construct comprising a photoreceptor-specific promoter guanine nucleotide-binding protein G subunit alpha-2 (GNAT2) promoter (SynP136) operably linked to a nucleic acid molecule encoding green fluorescent protein for use as a control in FIG. 4A.

FIGS. 5A-5B are fluorescent microscopic images of contralateral retinas from an rd10 homozygous mouse showing an increase of cone cells resulting from AAV-mediated expression of TXNIP using a RedO promoter (FIG. 5b), compared to an H2BGFP-only control (FIG. 5a).

FIG. 6 is a graph comparing the optomotor test results from rd10 homozygous mice with AAV-mediated expression of TXNIP driven by a human RedO promoter as compared to control rd10 mice.

FIGS. 7A-7B are immunohistochemical photomicrograph images of contralateral retinas from a wild-type mouse showing the up-regulation of LDHB expression (light grey) after subretinal administration of a composition comprising a RedO promoter operably linked to a nucleic acid molecule enclosing TXNIP (7B) as compared to a control (7A).

FIG. 8A is a scatter-plot distribution graph of ½ radius cone cell counts from retinas of rd1 homozygous mice following subretinal administration of an AAV expression construct comprising an siRNA molecule targeting lactate dehydrogenase B (LDHB) as compared to a control (NC).

FIG. 8B are schematics of an exemplary AAV expression construct comprising an siRNA molecule targeting lactate dehydrogenase (LDHB) (bottom) and control AAV constructs used in FIG. 8A.

FIG. 9A is a scatter-plot distribution graph of ½ radius cone cell counts from retinas of rd1 homozygous mice following subretinal administration of an AAV expression construct comprising a photoreceptor-specific promoter, human red opsin (RedO) operably linked to a nucleotic acid molecule encoding thioredoxin-interacting protein (TXNIP); or an AAV expression construct comprising a photoreceptor-specific promoter, human red opsin (RedO) operably linked to a nucleotic acid molecule encoding thioredoxin-interacting protein (TXNIP) and an AAV expression construct comprising an siRNA molecule targeting lactate dehydrogenase B (LDHB); or a control AAV construct (RO-NC).

FIG. 9B are schematic illustrations of exemplary expression cassettes encoding used in FIG. 9A.

FIG. 10 depicts an exemplary vector map of an exemplary AAV vector of the invention comprising a RedO promoter and a nucleic acid molecule encoding thioredoxin-interacting protein (TXNIP).

FIGS. 11A-11H depict the nucleotide sequence of the exemplary vector map of an exemplary AAV vector of the invention depicted in FIG. 10. FIGS. 11A-11H disclose the full-length nucleotide sequence as SEQ ID NO: 26, the protein sequences as SEQ ID NOS 27-29 and 29-36 and the primer sequences as SEQ ID NOS 37-38, all respectively, in order of appearance.

FIG. 12 depicts an exemplary vector map of an exemplary AAV vector of the invention comprising a SynPV1 promoter and a nucleic acid molecule encoding thioredoxin-interacting protein (TXNIP).

FIGS. 13A-13F depict the nucleotide sequence of the exemplary vector map of an exemplary AAV vector of the invention depicted in FIG. 12. FIGS. 13A-13F disclose the full-length nucleotide sequence as SEQ ID NO: 39, the protein sequences as SEQ ID NOS 40, 29, 29-30, 41-43 and 43-44 and the primer sequences as SEQ ID NOS 45-48, all respectively, in order of appearance.

FIG. 14 depicts an exemplary vector map of an exemplary AAV vector of the invention comprising a RedO promoter and a nucleic acid molecule encoding lactate dehydrogenase B (LDHB).

FIGS. 15A-15H depict the nucleotide sequence of the exemplary vector map of an exemplary AAV vector of the invention depicted in FIG. 14. FIGS. 15A-15H disclose the full-length nucleotide sequence as SEQ ID NO: 49, the protein sequences as SEQ ID NOS 27-28, 50-51, 51-53 and 32-36 and the primer sequences as SEQ ID NOS 54-59 and 48, all respectively, in order of appearance.

### DETAILED DESCRIPTION OF THE INVENTION

The present invention is based, at least in part on the discovery of mutation-independent compositions and methods of treatment for subjects having RP.

More specifically, it has surprisingly been discovered that intraocular delivery of AAV comprising thioredoxin interacting protein (TXNIP) prolongs survival of cones in RP-mutant mice. Even more surprising, this TXNIP-mediated effect was only observed when TXNIP was specifically expressed in cones. It has also surprisingly been discovered that overexpression of TXNIP causes up-regulation of lactate dehydrogenase B (LDHB) in RP cones and, further, that LDHB expression is necessary for the TXNIP-mediated rescue of cones.

Accordingly, the present invention provides compositions, e.g., pharmaceutical compositions, which include a recombinant adeno-associated virus (AAV) vector, and methods of treating a subject having a degenerative ocular disorder, e.g., retinitis pigmentosa.

Various aspects of the invention are described in further detail in the following subsections:

### I. Definitions

As used herein, each of the following terms has the meaning associated with it in this section.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to”.

The term “or” is used herein to mean, and is used interchangeably with, the term “and/or,” unless context clearly indicates otherwise.

As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA. A nucleic acid molecule used in the methods of the present invention can be isolated using standard molecular biology techniques. Using all or portion of a nucleic acid sequence of interest as a hybridization probe, nucleic acid molecules can be isolated using standard hybridization and cloning techniques (e.g., as described in Sambrook, J., Fritsh, E. F., and Maniatis, T. Molecular Cloning. A Laboratory Manual. 2nd, ed., Cold Spring Harbor Laboratory, Cold Spring Harbor Laboratory Press, Cold Spring Harbor, N.Y., 1989).

An “isolated” nucleic acid molecule is one which is separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid molecule is free of sequences which naturally flank the nucleic acid molecule (i.e., sequences located at the 5′ and 3′ ends of the nucleic acid molecule) in the genomic DNA of the organism from which the nucleic acid molecule is derived.

A nucleic acid molecule for use in the methods of the invention can also be isolated by the polymerase chain reaction (PCR) using synthetic oligonucleotide primers designed based upon the sequence of a nucleic acid molecule of interest. A nucleic acid molecule used in the methods of the invention can be amplified using cDNA, mRNA or, alternatively, genomic DNA as a template and appropriate oligonucleotide primers according to standard PCR amplification techniques. Furthermore, oligonucleotides corresponding to nucleotide sequences of interest can be prepared by standard synthetic techniques, e.g., using an automated DNA synthesizer.

The nucleic acids for use in the methods of the invention can also be prepared, e.g., by standard recombinant DNA techniques. A nucleic acid of the invention can also be chemically synthesized using standard techniques. Various methods of chemically synthesizing polydeoxynucleotides are known, including solid-phase synthesis which has been automated in commercially available DNA synthesizers (See e.g., Itakura et al. U.S. Pat. No. 4,598,049; Caruthers et al. U.S. Pat. No. 4,458,066; and Itakura U.S. Pat. Nos. 4,401,796 and 4,373,071, incorporated by reference herein).

As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes or nucleic acid molecules to which they are operatively linked and are referred to as “expression vectors” or “recombinant expression vectors.”. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals. In some embodiments, “expression vectors” are used in order to permit pseudotyping of the viral envelope proteins.

Expression vectors are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses, adeno-associated viruses, lentiviruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells, those which are constitutively active, those which are inducible, and those which direct expression of the nucleotide sequence only in certain host cells (e.g., tissue-specific regulatory sequences). The expression vectors of the invention can be introduced into host cells to thereby produce proteins or portions thereof, including fusion proteins or portions thereof, encoded by nucleic acids as described herein.

The terms “transformation,” “transfection,” and “transduction” refer to introduction of a nucleic acid, e.g., a viral vector, into a recipient cell.

As used herein, the term “subject” includes warm-blooded animals, preferably mammals, including humans. In a preferred embodiment, the subject is a primate. In an even more preferred embodiment, the primate is a human.

As used herein, the various forms of the term “modulate” are intended to include stimulation (e.g., increasing or upregulating a particular response or activity) and inhibition (e.g., decreasing or downregulating a particular response or activity).

As used herein, the term “contacting” (i.e., contacting a cell with an agent) is intended to include incubating the agent and the cell together in vitro (e.g., adding the agent to cells in culture) or administering the agent to a subject such that the agent and cells of the subject are contacted in vivo. The term “contacting” is not intended to include exposure of cells to an agent that may occur naturally in a subject (i.e., exposure that may occur as a result of a natural physiological process).

As used herein, the term “administering” to a subject includes dispensing, delivering or applying a composition of the invention to a subject by any suitable route for delivery of the composition to the desired location in the subject, including delivery by intraocular administration or intravenous administration. Alternatively or in combination, delivery is by the topical, parenteral or oral route, intracerebral injection, intramuscular injection, subcutaneous/intradermal injection, intravenous injection, buccal administration, transdermal delivery and administration by the rectal, colonic, vaginal, intranasal or respiratory tract route.

As used herein, the term “degenerative ocular disorder” refers generally to a disorder of the retina. In one embodiment, the degenerative ocular disorder is associated with death, of cone cells, and/or rod cells. Moreover, in a particular embodiment, a degenerative ocular disorder is not associated with blood vessel leakage and/or growth, for example, as is the case with diabetic retinopathy, but, instead is characterized primarily by reduced viability of cone cells and/or rod cells. In certain embodiments, the degenerative ocular disorder is a genetic or inherited disorder. In a particular embodiment, the degenerative ocular disorder is retinitis pigmentosa. In another embodiment, the degenerative ocular disorder is age-related macular degeneration. In another embodiment, the degenerative ocular disorder is cone-rod dystrophy. In another embodiment, the degenerative ocular disorder is rod-cone dystrophy. In other embodiments, the degenerative ocular disorder is not associated with blood vessel leakage and/or growth. In certain embodiments, the degenerative ocular disorder is not associated with diabetes and/or diabetic retinopathy. In further embodiments, the degenerative ocular disorder is not NARP (neuropathy, ataxia, and retinitis pigmentosa). In yet further embodiments, the degenerative ocular disorder is not a neurological disorder. In certain embodiments, the degenerative ocular disorder is not a disorder that is associated with a compromised optic nerve and/or disorders of the brain. In the foregoing embodiments, the degenerative ocular disorder is associated with a compromised photoreceptor cell, and is not a neurological disorder.

As used herein, the term “retinitis pigmentosa” or “RP” is known in the art and encompasses a disparate group of genetic disorders of rods and cones. Retinitis pigmentosa generally refers to retinal degeneration often characterized by the following manifestations: night blindness, progressive loss of peripheral vision, eventually leading to total blindness; ophthalmoscopic changes consist in dark mosaic-like retinal pigmentation, attenuation of the retinal vessels, waxy pallor of the optic disc, and in the advanced forms, macular degeneration. In some cases there can be a lack of pigmentation. Retinitis pigmentosa can be associated to degenerative opacity of the vitreous body, and cataract. Family history is prominent in retinitis pigmentosa; the pattern of inheritance may be autosomal recessive, autosomal dominant, or X-linked; the autosomal recessive form is the most common and can occur sporadically.

As used herein, the terms “Cone-Rod Dystrophy” or “CRD” and “Rod-Cone Dystrophy” or “RCD” refer to art recognized inherited progressive diseases that cause deterioration of the cone and rod photoreceptor cells and often result in blindness. CRD is characterized by reduced viability or death of cone cells followed by reduced viability or death of rod cells. By contrast, RCD is characterized by reduced viability or death of rod cells followed by reduced viability or death of cone cells.

As used herein, the term “age-related macular degeneration” also referred to as “macular degeneration” or “AMD”, refers to the art recognized pathological condition which causes blindness amongst elderly individuals. Age related macular degeneration includes both wet and dry forms of AMD. The dry form of AMD, which accounts for about 90 percent of all cases, is also known as atrophic, nonexudative, or drusenoid (age-related) macular degeneration. With the dry form of AMD, drusen typically accumulate in the retinal pigment epithelium (RPE) tissue beneath/within the Bruch's membrane. Vision loss can then occur when drusen interfere with the function of photoreceptors in the macula. The dry form of AMD results in the gradual loss of vision over many years. The dry form of AMD can lead to the wet form of AMD. The wet form of AMD, also known as exudative or neovascular (age-related) macular degeneration, can progress rapidly and cause severe damage to central vision. The macular dystrophies include Stargardt Disease, also known as Stargardt Macular Dystrophy or Fundus Flavimaculatus, which is the most frequently encountered juvenile onset form of macular dystrophy.

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease).

As used herein, the terms “treating” or “treatment” refer to a beneficial or desired result including, but not limited to, alleviation or amelioration of one or more symptoms, diminishing the extent of infection, stabilized (i.e., not worsening) state of infection, amelioration or palliation of the infectious state, whether detectable or undetectable. “Treatment” can also mean prolonging survival as compared to expected survival in the absence of treatment.

Various additional aspects of the methods of the invention are described in further detail in the following subsections.

### II. Compositions of the Invention

The present invention provides adeno-associated viral (AAV) expression cassettes, AAV expression cassettes present in AAV vectors, and AAV vectors comprising a recombinant viral genome which include an expression cassette.

Accordingly, in one aspect the present invention provides compositions comprising an adeno-associated virus (AAV) expression cassette, the expression cassette comprising a promoter and a nucleic acid molecule encoding thioredoxin-interacting protein (TXNIP).

In another aspect, the present invention provides compositions comprising an adeno-associated virus (AAV) expression cassette, the expression cassette comprising a photoreceptor-specific (PR-specific) promoter and a nucleic acid molecule encoding lactate dehydrogenase B (LDHB).

In a further aspect, the present invention provides compositions comprising an adeno-associated virus (AAV) expression cassette, the expression cassette comprising a photoreceptor-specific (PR-specific) promoter and a nucleic acid molecule encoding thioredoxin-interacting protein (TXNIP) and a nucleic acid molecule encoding lactate dehydrogenase B (LDHB). In some embodiments, the expression cassette comprises a linker nucleic acid molecule between the nucleic acid molecule encoding TXNIP and the nucleic acid molecule encoding LDHB.

In another aspect, the present invention provides compositions comprising a first adeno-associated virus (AAV) expression cassette, the expression cassette comprising a first photoreceptor-specific (PR-specific) promoter and a nucleic acid molecule encoding thioredoxin-interacting protein (TXNIP), and a second adeno-associated virus (AAV) expression cassette, the expression cassette comprising a second photoreceptor-specific (PR-specific) promoter and a nucleic acid molecule encoding lactate dehydrogenase B (LDHB).

In some embodiments, the promoter is a cone-specific promoter. In some embodiments, the cone-specific promoter is a human red opsin (RedO) promoter. In other embodiments, the promoter is a guanine nucleotide-binding protein G subunit alpha-2 (GNAT2) promoter.

In some embodiments, the expression cassettes of the invention further comprise an intron, such as an intron between the promoter and the nucleic acid molecule encoding TXNIP.

In some embodiments of the invention, the expression cassettes of the invention further comprise expression control sequences including, but not limited to, appropriate transcription sequences (i.e. initiation, termination, and enhancer), efficient RNA processing signals (e.g. splicing and polyadenylation (polyA) signals), sequences that stabilize cytoplasmic mRNA, sequences that code for a transcriptional enhancer, sequences that code for a posttranscriptional enhancer, sequences that enhance translation efficiency (i.e. Kozak consensus sequence), sequences that enhance protein stability, and when desired, sequences that enhance secretion of the encoded product.

The terms “adeno-associated virus”, “AAV virus”, “AAV virion”, “AAV viral particle”, and “AAV particle”, as used interchangeably herein, refer to a viral particle composed of at least one AAV capsid protein (preferably by all of the capsid proteins of a particular AAV serotype) and an encapsidated polynucleotide AAV genome. If the particle comprises a heterologous polynucleotide (i.e. a polynucleotide other than a wild-type AAV genome such as a transgene to be delivered to a mammalian cell) flanked by the AAV inverted terminal repeats (ITRs), it is typically referred to as an “AAV vector particle.”

AAV viruses belonging to the genus Dependovirus of the Parvoviridae family and, as used herein, include any serotype of the over 100 serotypes of AAV viruses known. In general, serotypes of AAV viruses have genomic sequences with a significant homology at the level of amino acids and nucleic acids, provide an identical series of genetic functions, produce virions that are essentially equivalent in physical and functional terms, and replicate and assemble through practically identical mechanisms.

The AAV genome is approximately 4.7 kilobases long and is composed of single-stranded deoxyribonucleic acid (ssDNA) which may be either positive- or negative-sensed. The genome comprises inverted terminal repeats (ITRs) at both ends of the DNA strand, and two open reading frames (ORFs): rep and cap. The rep frame is made of four overlapping genes encoding Rep proteins required for the AAV life cycle. The cap frame contains overlapping nucleotide sequences of capsid proteins: VP1, VP2 and VP3, which interact together to form a capsid of an icosahedral symmetry. See Carter B, Adeno-associated virus and adeno-associated virus vectors for gene delivery, Lassie D, et ah, Eds., “Gene Therapy: Therapeutic Mechanisms and Strategies” (Marcel Dekker, Inc., New York, N.Y., US, 2000) and Gao G, et al, J. Virol. 2004; 78(12):6381-6388.

The term “AAV vector” or “AAV construct” refers to a vector derived from an adeno-associated virus serotype, including without limitation, AAV-1, AAV-2, AAV-3, AAV-4, AAV-5, AAV6, AAV7, AAV8, and AAV9. “AAV vector” refers to a vector that includes AAV nucleotide sequences as well as heterologous nucleotide sequences. AAV vectors require only the 145 base terminal repeats in cis to generate virus. All other viral sequences are dispensable and may be supplied in trans (Muzyczka (1992) Curr. Topics Microbiol. Immunol. 158:97-129). Typically, the rAAV vector genome will only retain the inverted terminal repeat (ITR) sequences so as to maximize the size of the transgene that can be efficiently packaged by the vector. The ITRs need not be the wild-type nucleotide sequences, and may be altered, e.g., by the insertion, deletion or substitution of nucleotides, as long as the sequences provide for functional rescue, replication and packaging.

In particular embodiments, the AAV vector is an AAV2, AAV2.7m8, AAV2/5 or AAV2/8 vector. Suitable AAV vectors are described in, for example, U.S. Pat. No. 7,056,502 and Yan et al. (2002) J. Virology 76(5):2043-2053, the entire contents of which are incorporated herein by reference.

Such AAV vectors can be replicated and packaged into infectious viral particles when present in a host cell that has been transfected with a vector encoding and expressing rep and cap gene products (i.e. AAV Rep and Cap proteins), and wherein the host cell has been transfected with a vector which encodes and expresses a protein from the adenovirus open reading frame E4orf6.

The term “cap gene” or “AAV cap gene”, as used herein, refers to a gene that encodes a Cap protein. The term “Cap protein”, as used herein, refers to a polypeptide having at least one functional activity of a native AAV Cap protein (e.g. VP1, VP2, VP3). Examples of functional activities of Cap proteins (e.g. VP1, VP2, VP3) include the ability to induce formation of a capsid, facilitate accumulation of single-stranded DNA, facilitate AAV DNA packaging into capsids (i.e. encapsidation), bind to cellular receptors, and facilitate entry of the virion into host.

The term “capsid”, as used herein, refers to the structure in which the viral genome is packaged. A capsid consists of several oligomeric structural subunits made of proteins. For instance, AAV have an icosahedral capsid formed by the interaction of three capsid proteins: VP1, VP2 and VP3.

The term “genes providing helper functions”, as used herein, refers to genes encoding polypeptides which perform functions upon which AAV is dependent for replication (i.e. “helper functions”). The helper functions include those functions required for AAV replication including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus. Helper functions include, without limitation, adenovirus E1, E2a, VA, and E4 or herpesvirus UL5, UL8, UL52, and UL29, and herpesvirus polymerase. In one embodiment, a helper function does not include adenovirus E1.

The term “rep gene” or “AAV rep gene”, as used herein, refers to a gene that encodes a Rep protein. The term “Rep protein”, as used herein, refers to a polypeptide having at least one functional activity of a native AAV Rep protein (e.g. Rep 40, 52, 68, 78). A “functional activity” of a Rep protein (e.g. Rep 40, 52, 68, 78) is any activity associated with the physiological function of the protein, including facilitating replication of DNA through recognition, binding and nicking of the AAV origin of DNA replication as well as DNA helicase activity. Additional functions include modulation of transcription from AAV (or other heterologous) promoters and site-specific integration of AAV DNA into a host chromosome.

The term “adeno-associated virus ITRs” or “AAV ITRs”, as used herein, refers to the inverted terminal repeats present at both ends of the DNA strand of the genome of an adeno-associated virus. The ITR sequences are required for efficient multiplication of the AAV genome. Another property of these sequences is their ability to form a hairpin. This characteristic contributes to its self-priming which allows the primase-independent synthesis of the second DNA strand. The ITRs have also shown to be required for efficient encapsidation of the AAV DNA combined with generation of fully assembled, deoxyribonuclease-resistant AAV particles.

The term “expression cassette”, as used herein, refers to a nucleic acid construct, generated recombinantly or synthetically, with a series of specified nucleic acid elements, which permit transcription of a particular nucleic acid in a target cell.

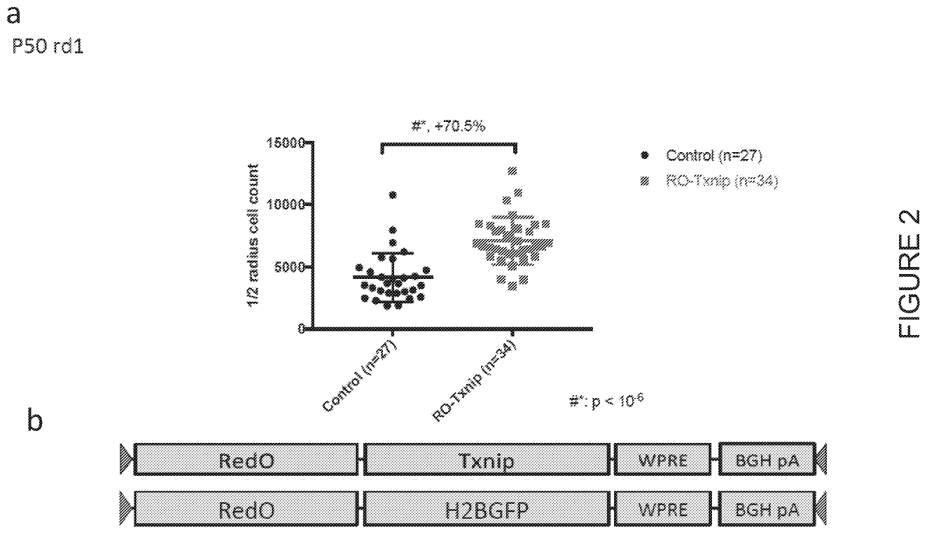

The expression cassettes of the invention include a promoter operably linked to a nucleic acid molecule encoding thioredoxin-interacting protein (TXNIP) and/or lactate dehydrogenase B (LDHB). Exemplary expression cassettes of the invention are depicted in FIGS. 2B, 3B, and 8B.

The term “promoter” as used herein refers to a recognition site of a DNA strand to which the RNA polymerase binds. The promoter forms an initiation complex with RNA polymerase to initiate and drive transcriptional activity. The complex can be modified by activating sequences termed “enhancers” or inhibitory sequences termed “silencers”.

Suitable promoters for use in the expression cassettes of the invention may be ubiquitous promoters, such as a CMV promoter or an SV40 promoter, but are preferably tissue-specific promoters, i.e., promoters that direct expression of a nucleic acid molecule preferentially in a particular cell type.

In one embodiment, a tissue-specific promoter for use in the present invention is a photoreceptor-specific (PR-specific) promoter. The PR-specific promoter may be a rod-specific promoter; a cone-specific promoter; or a rod- and cone-specific promoter. In one embodiment, a tissue-specific promoter for use in the present invention is a cone-specific promoter.

Suitable PR-specific promoters are known in the art and include, for example, a human red opsin, a guanine nucleotide-binding protein G subunit alpha-2 (GNAT2) promoter, a human rhodopsin promoter, a human rhodopsin kinase (RK) promoter, a G protein-coupled receptor kinase 1 (GRK1) promoter.

In certain embodiments, a suitable PR-specific promoter is a human red opsin (RedO) promoter.

As used interchangeably herein, the terms “human RO,” “red opsin,” “RedO,” “RO,” and “hRO” refer to Opsin 1, Long Wave Sensitive, also known as Red Cone Photoreceptor Pigment, Opsin 1 (Cone Pigments), Long-Wave-Sensitive, Cone Dystrophy 5 (X-Linked), Red-Sensitive Opsin, RCP, ROP, Long-Wave-Sensitive Opsin, Color Blindness, Protan, Red Cone Opsin, COD5, CBBm, and CBP. The nucleotide sequence of the genomic region containing the hRO gene (including the region upstream of the coding region of hRO which includes the hRO promoter region) is also known and may be found in, for example, GenBank Reference Sequence NG_009105.2 (SEQ ID NO: 8, the entire contents of which is incorporated herein by reference).

Suitable RedO promoters for use in the present invention include nucleic acid molecules which include nucleotides 452-2017 of SEQ ID NO:8 directly linked, i.e., containing no intervening sequences, to nucleotides 4541-5032 of SEQ ID NO:12; or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 452-2017 of SEQ ID NO:8 directly linked to nucleotides 4541-5032 of SEQ ID NO:8.

In one embodiment, the RedO promoter comprises the nucleotide sequence of SEQ ID NO:16, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of SEQ ID NO:16.

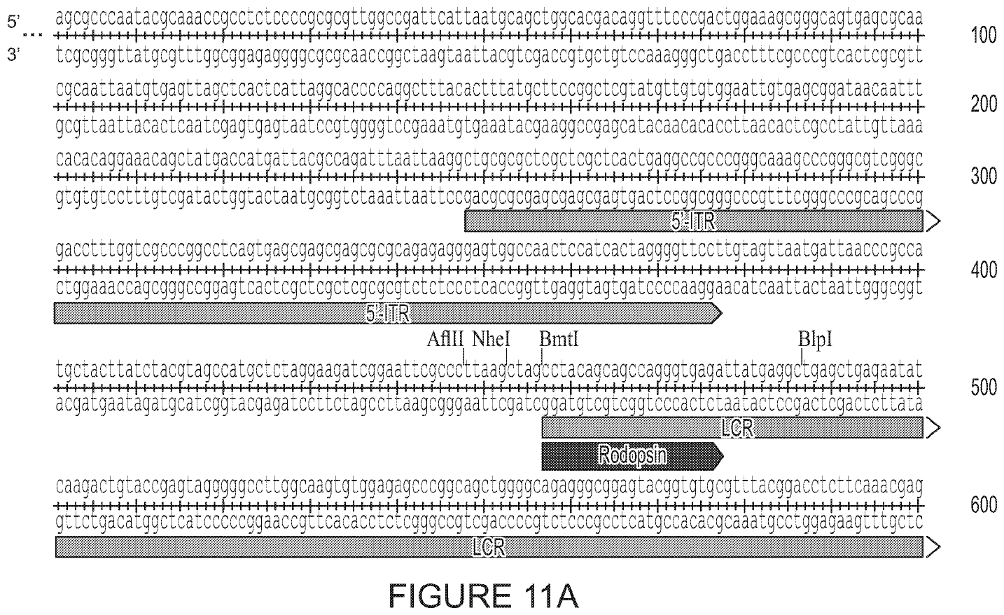

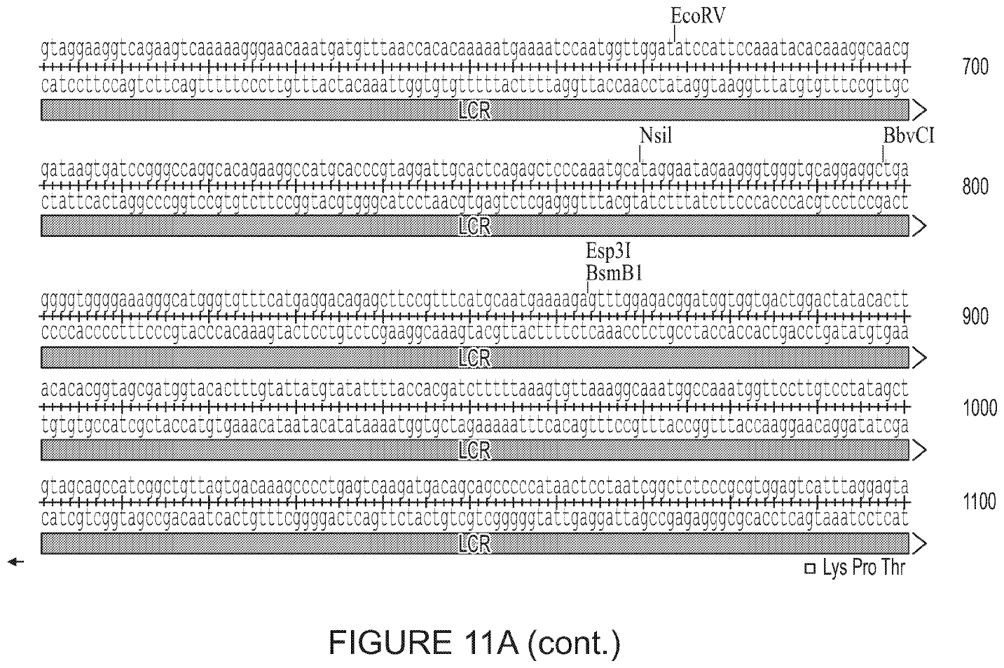

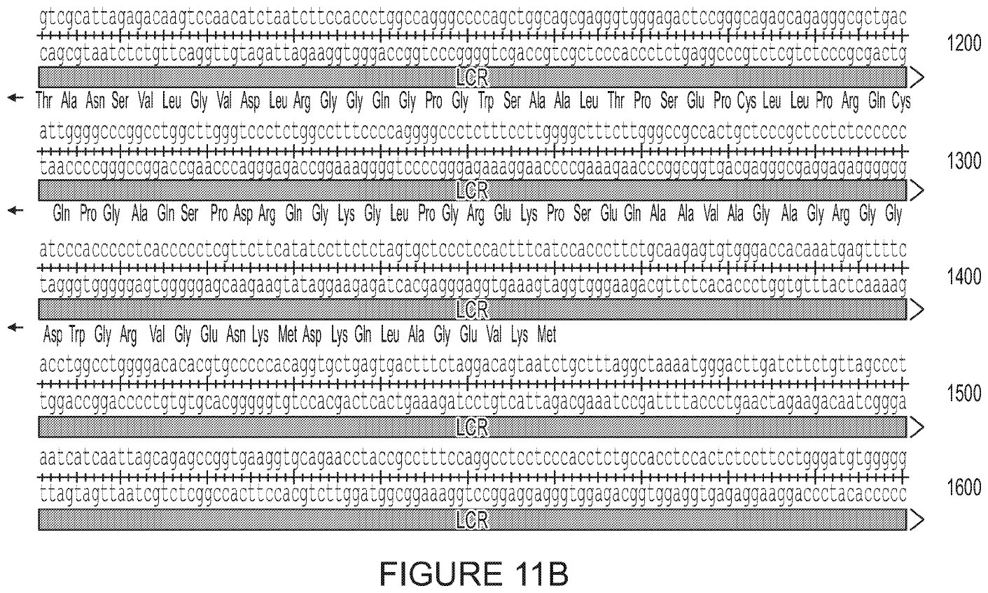

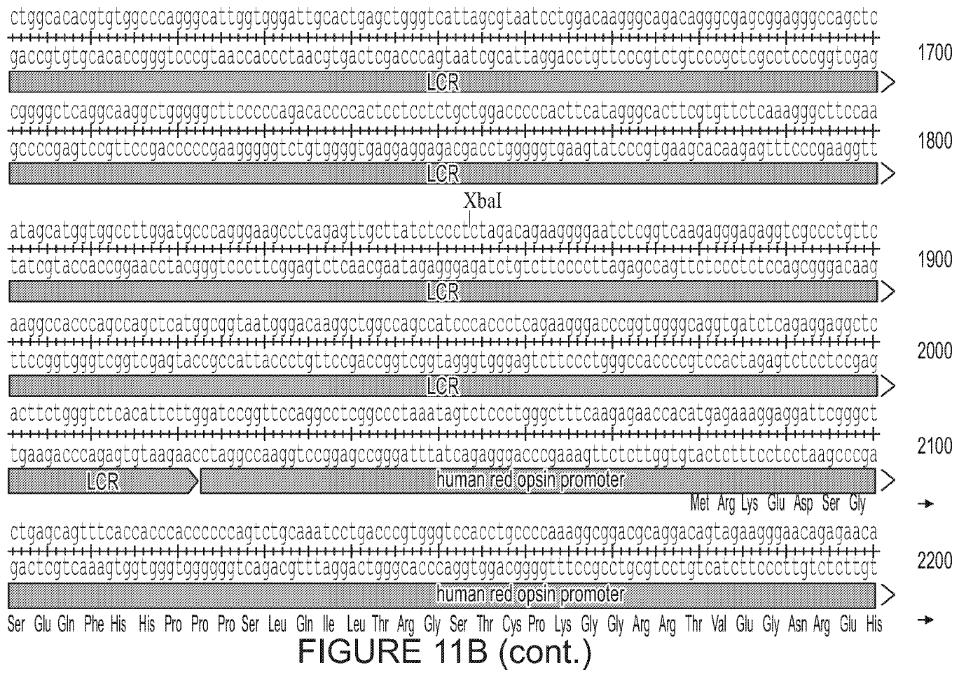

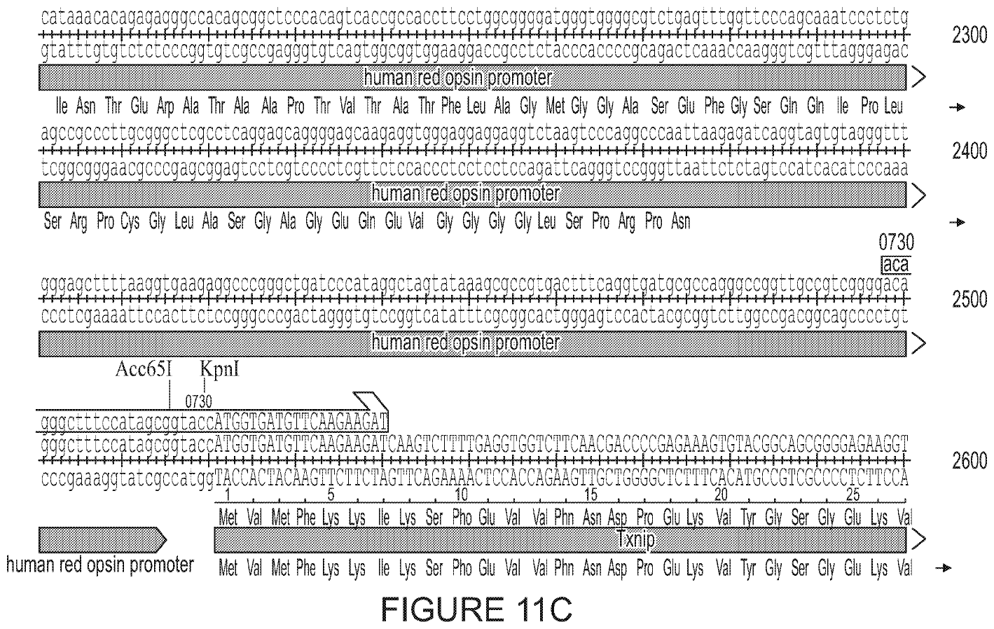

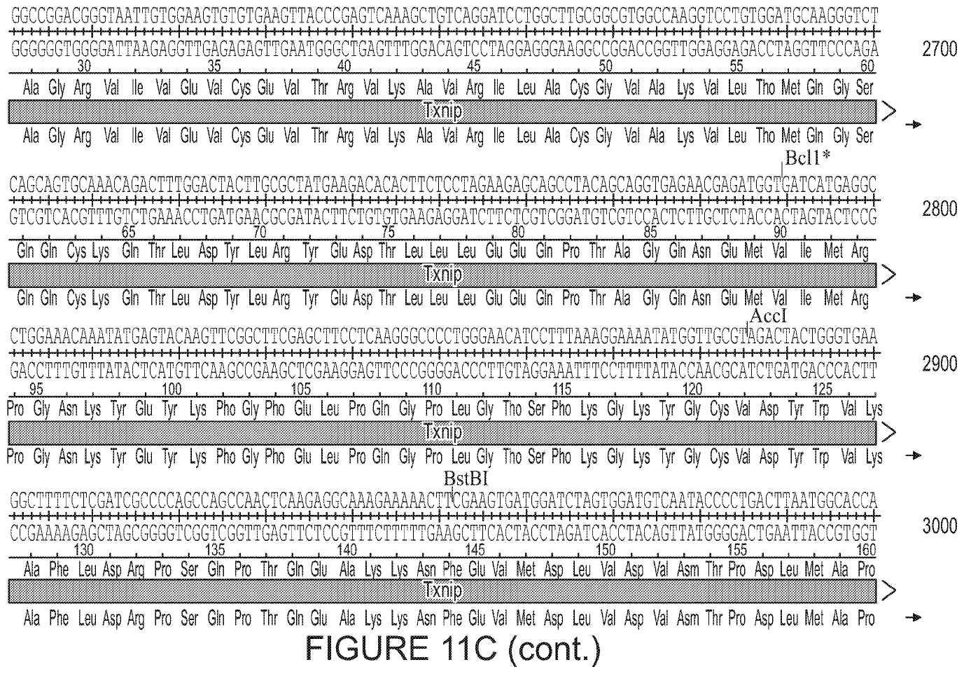

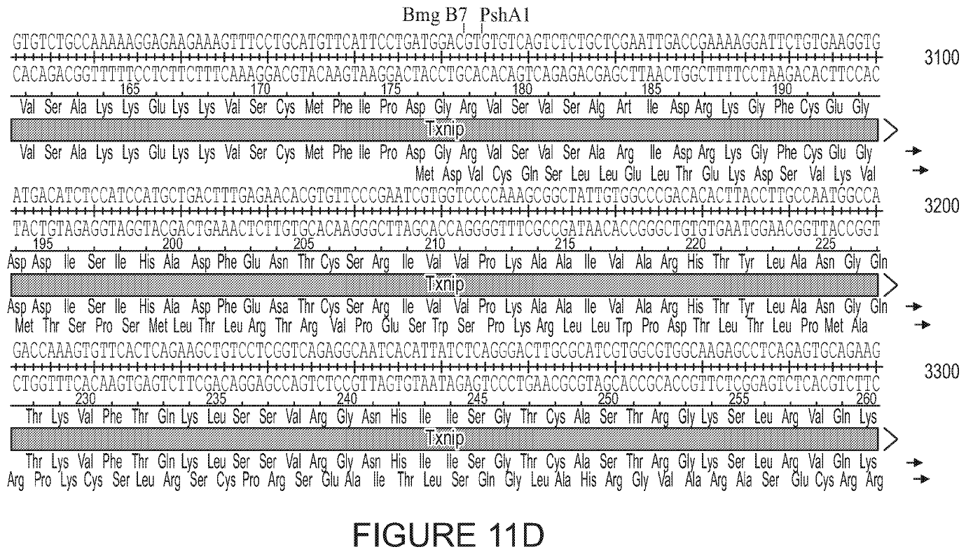

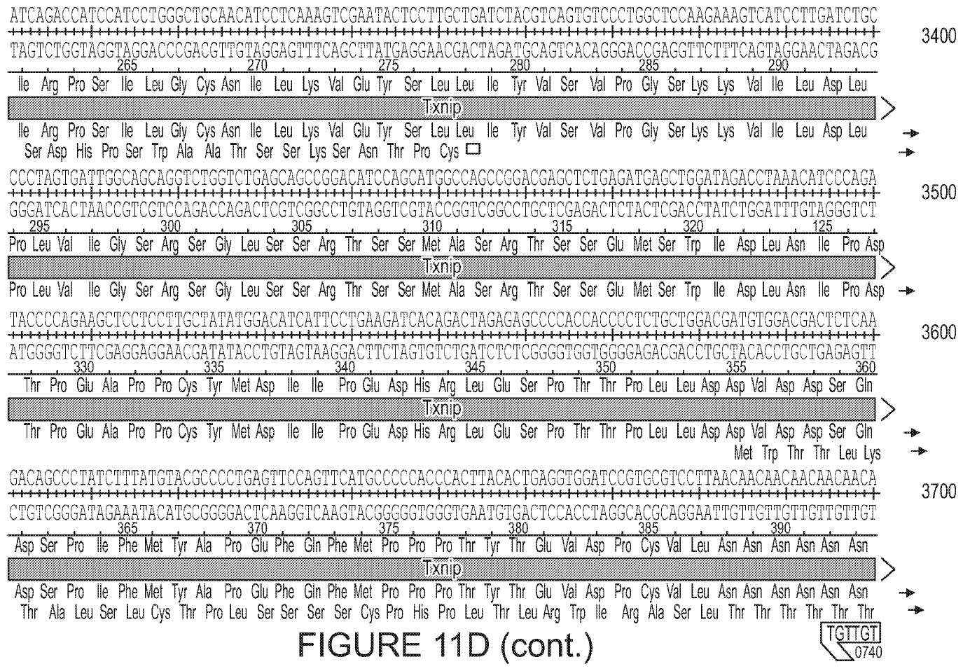

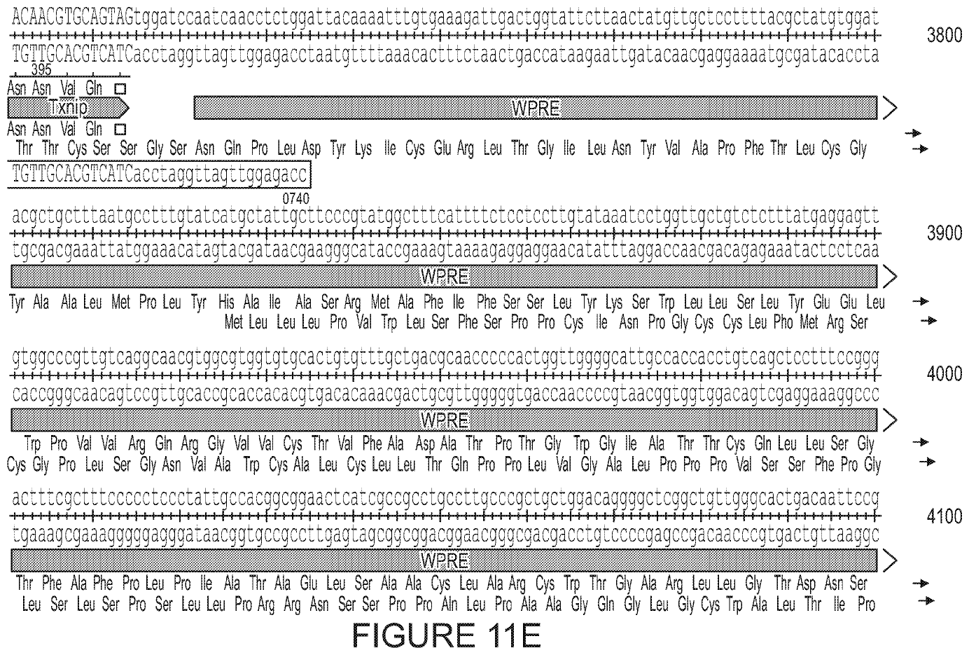

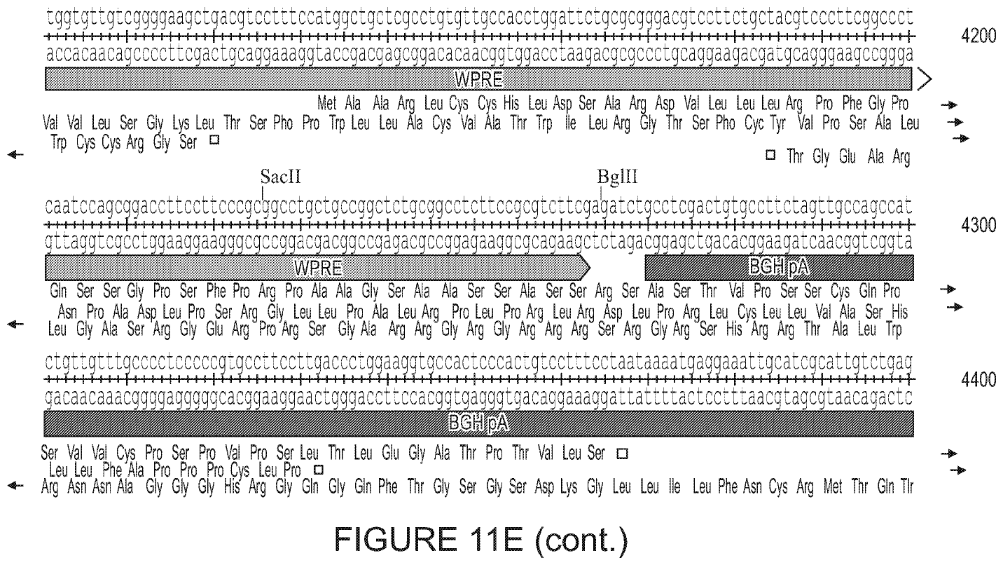

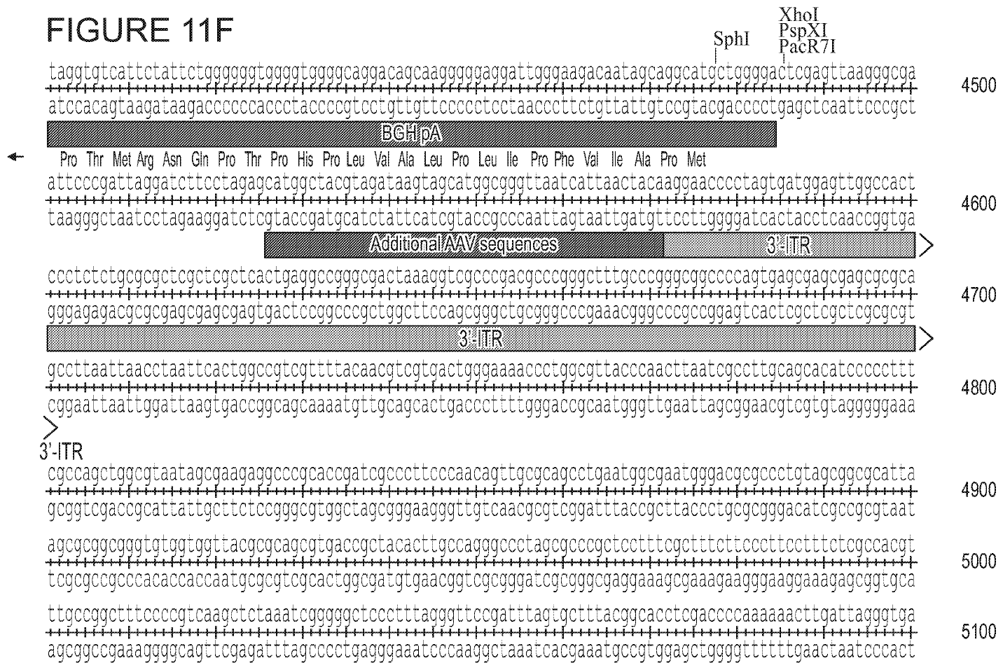

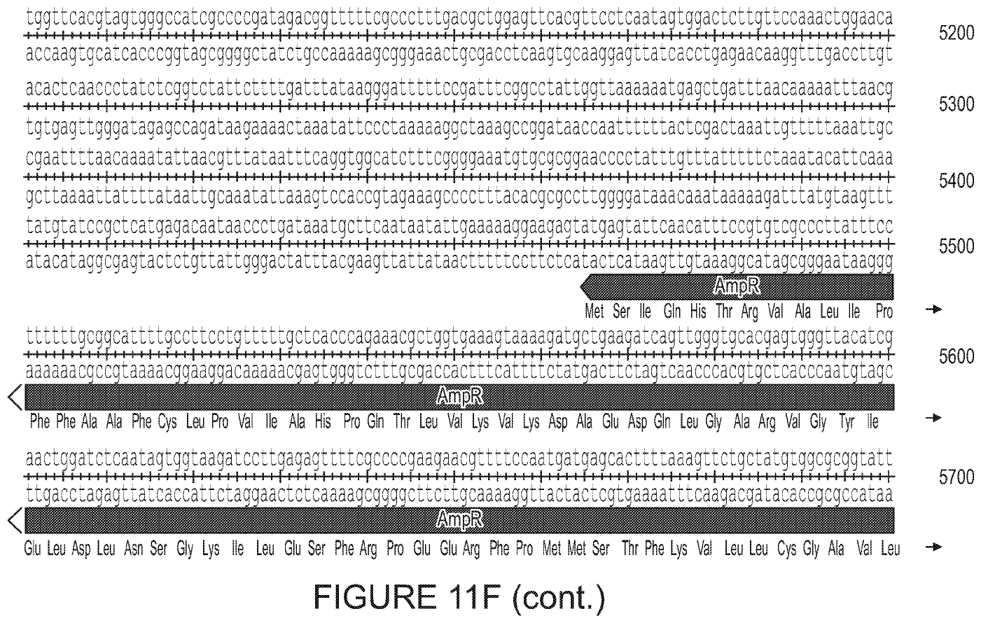

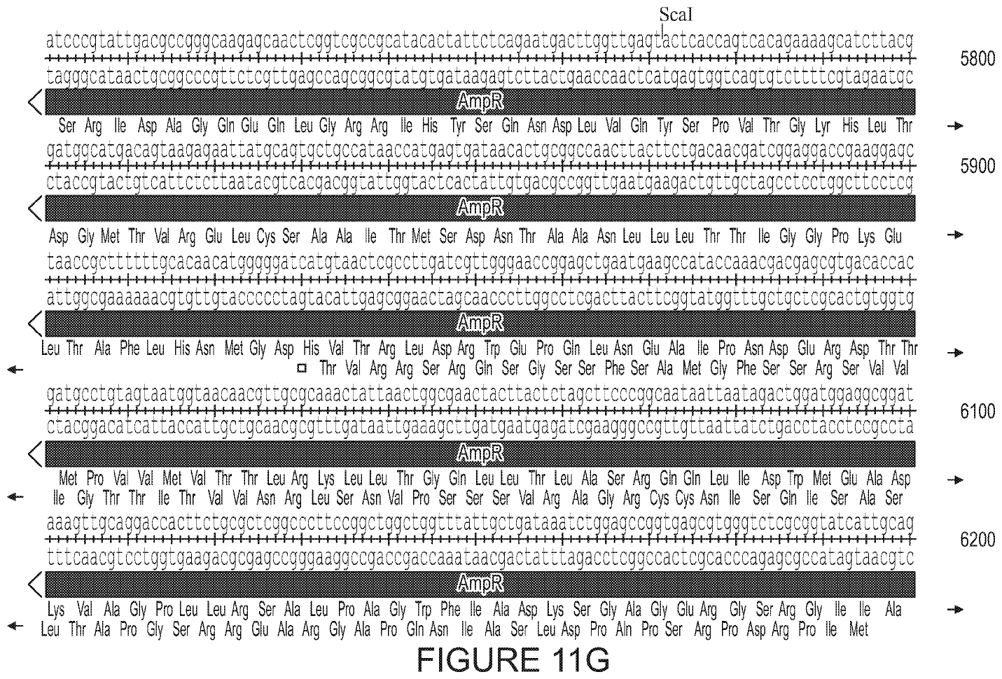

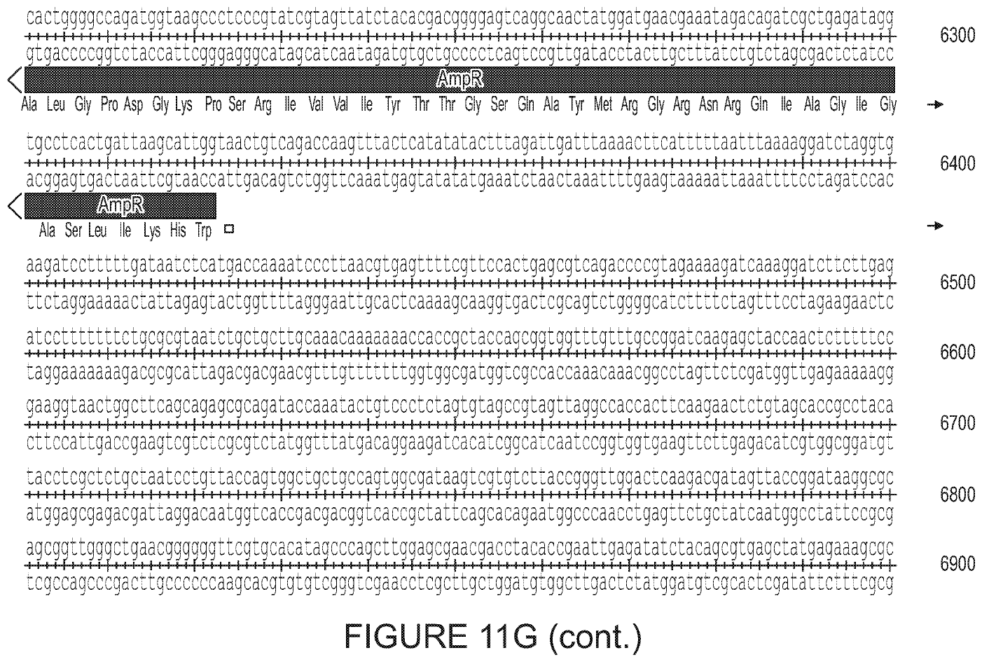

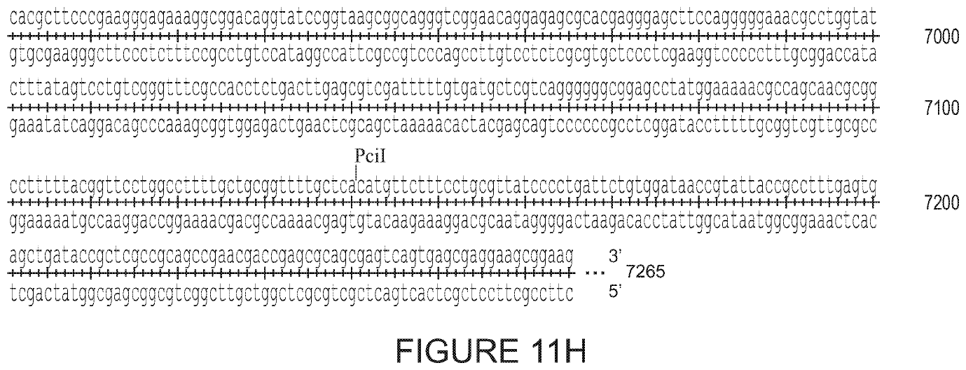

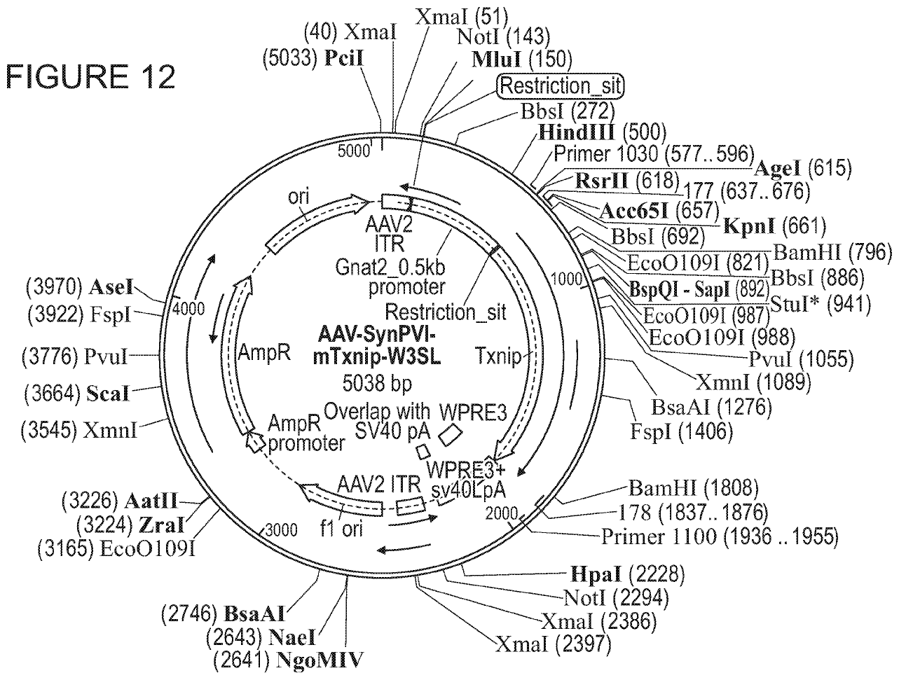

In one embodiment, the RedO promoter comprises nucleotides 457-2514 of the nucleotide sequence depicted in FIG. 11 (SEQ ID NO:26), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 457-2514 of the nucleotide sequence depicted in FIG. 11 (SEQ ID NO:26).

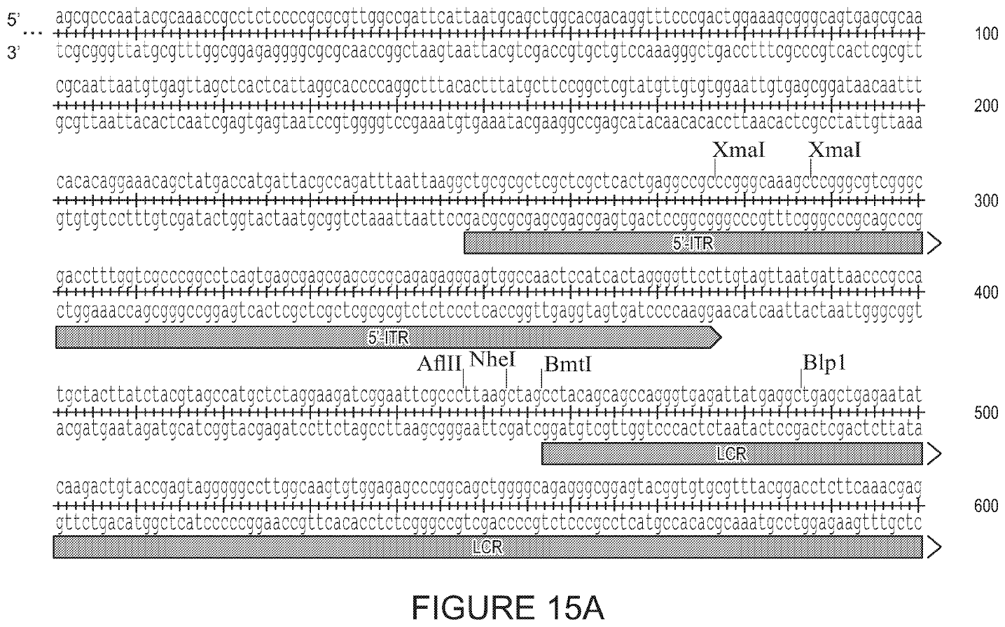

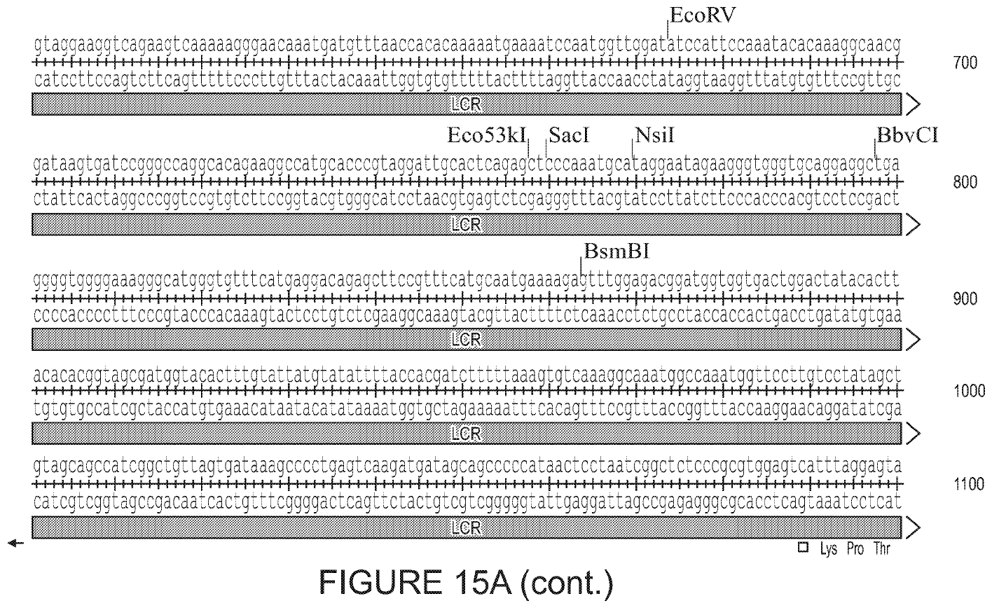

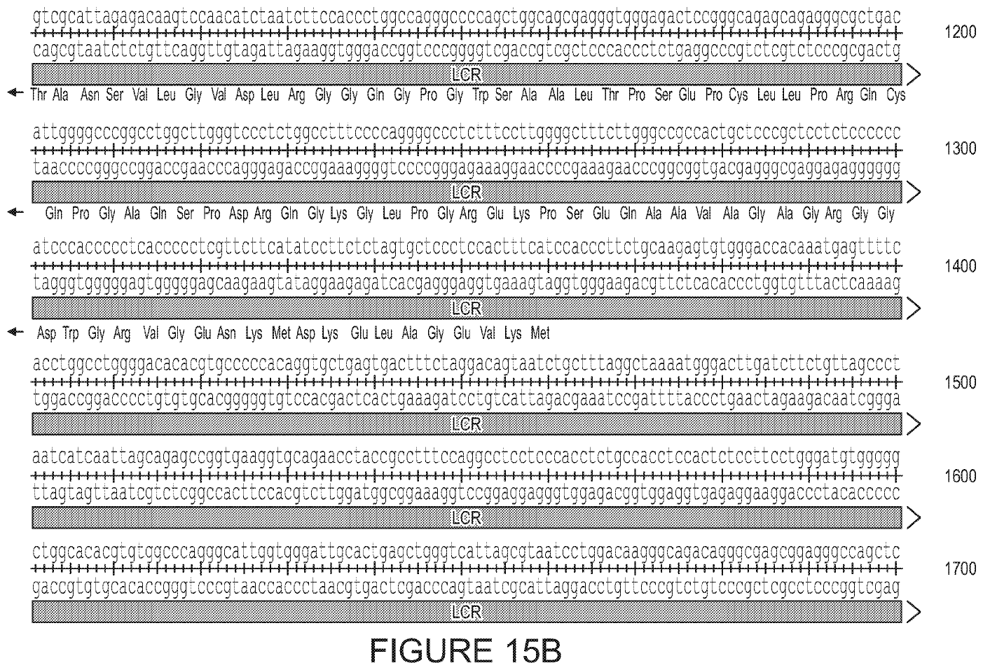

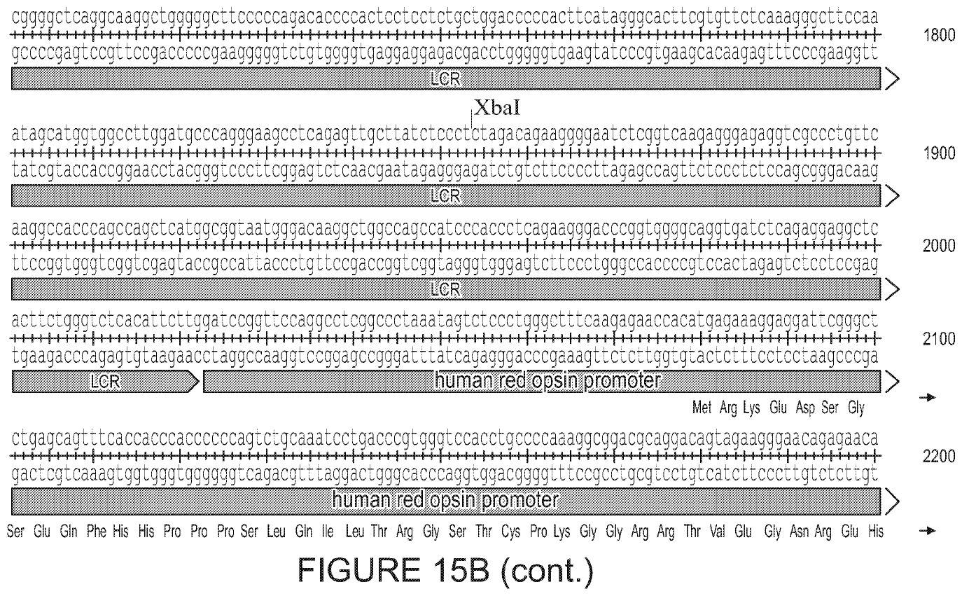

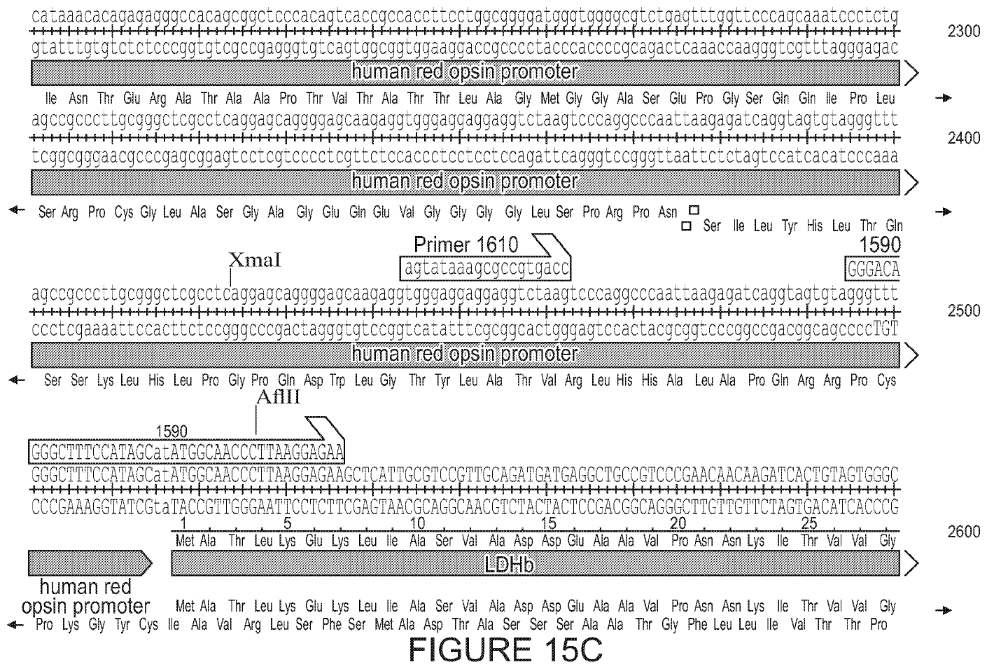

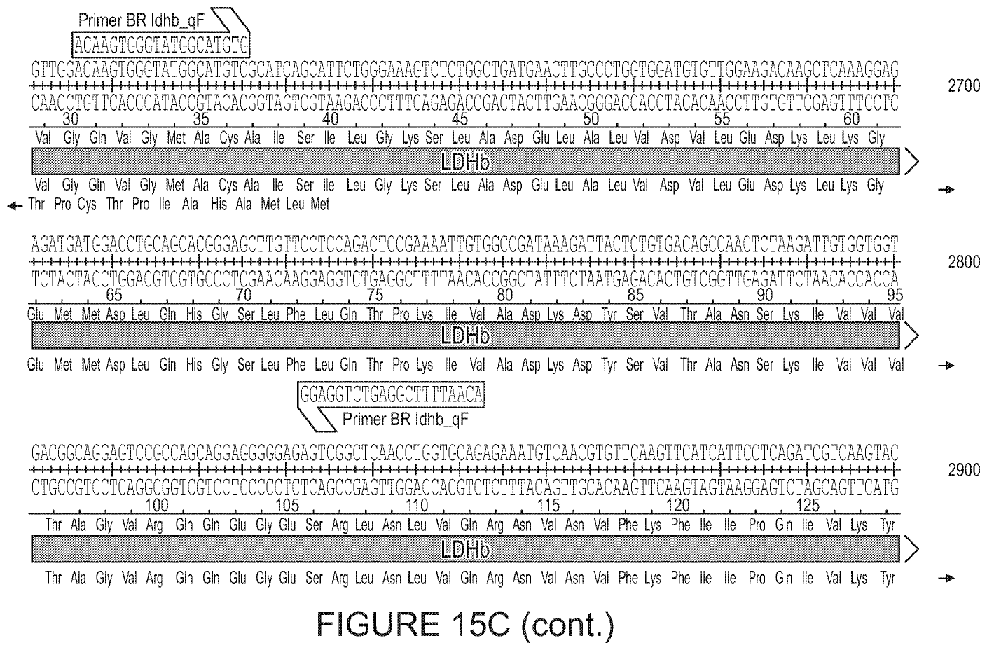

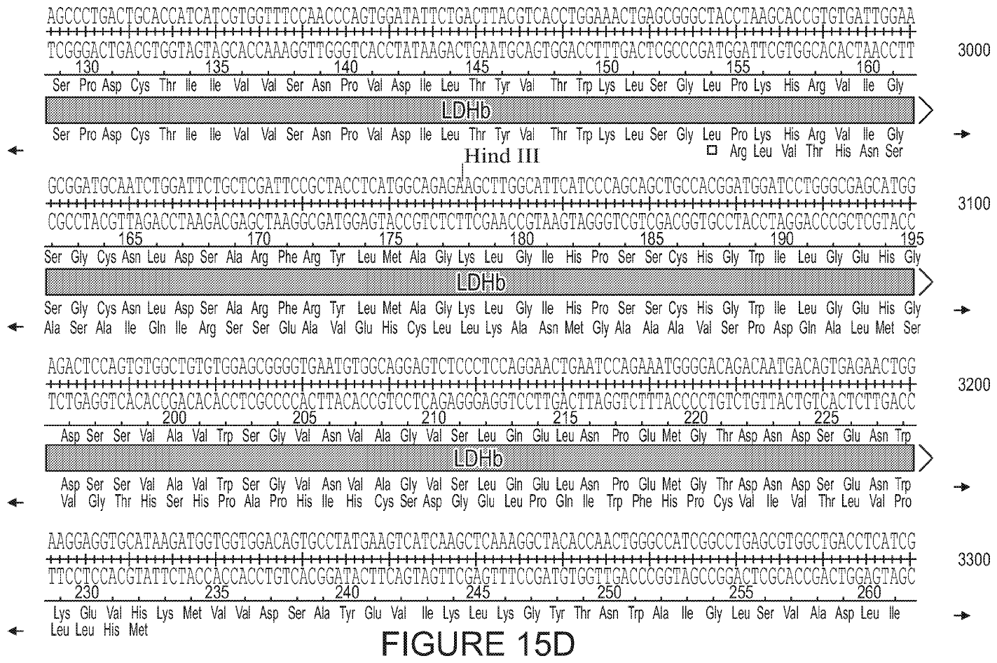

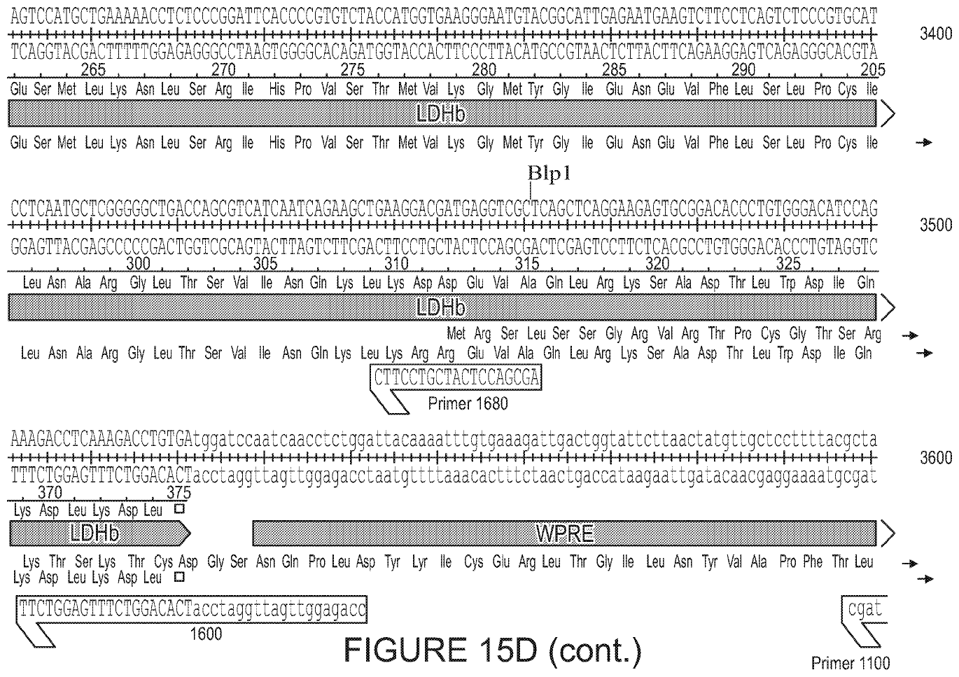

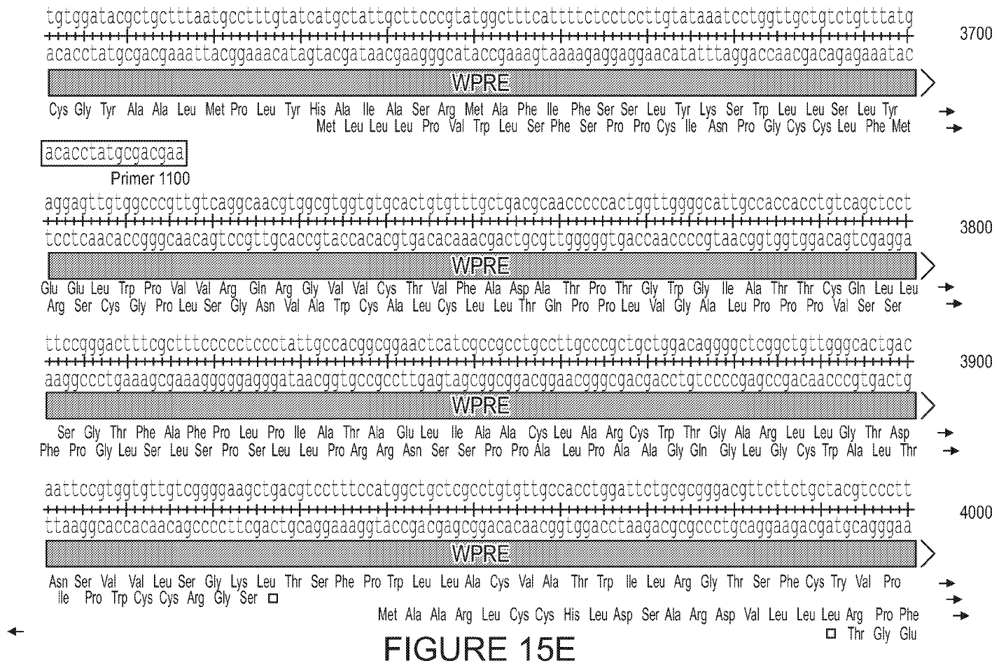

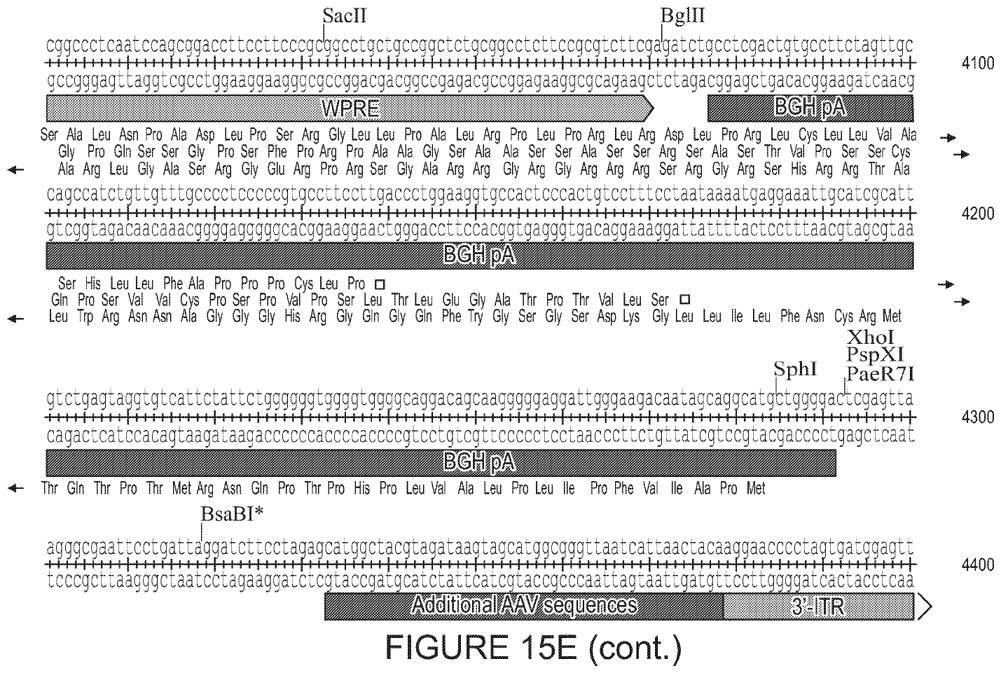

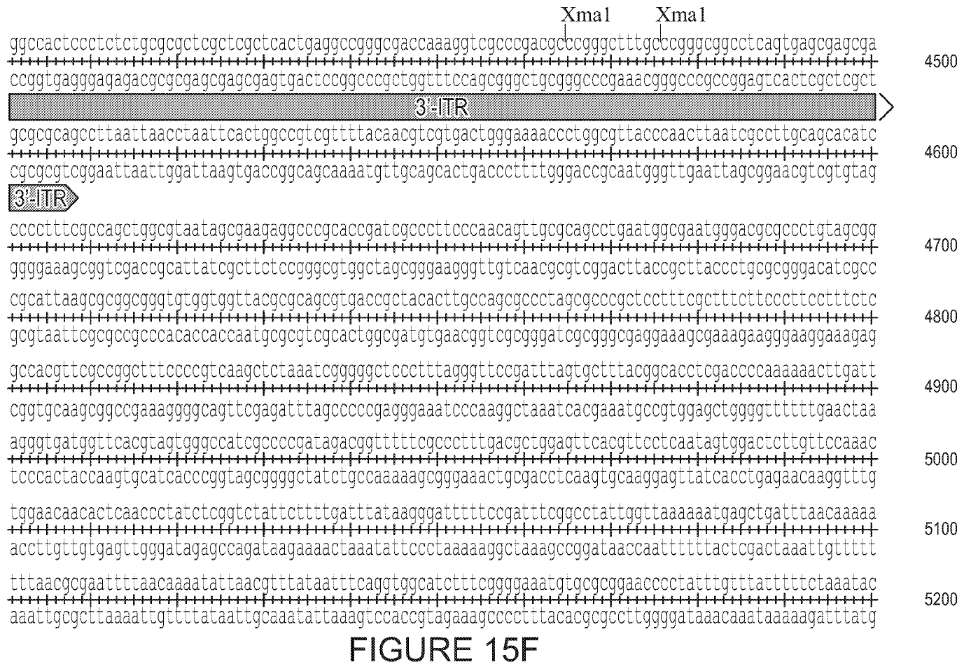

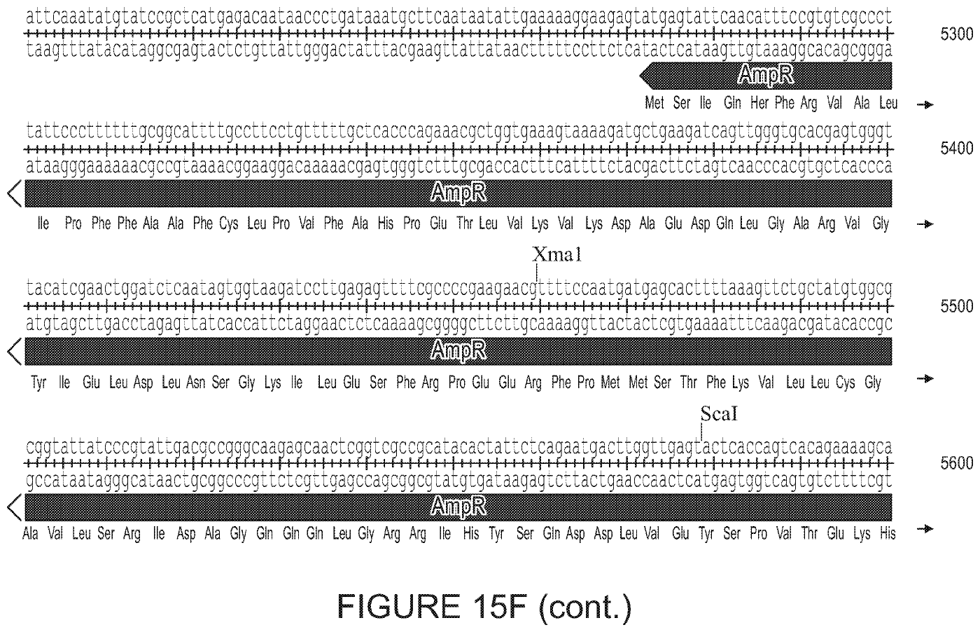

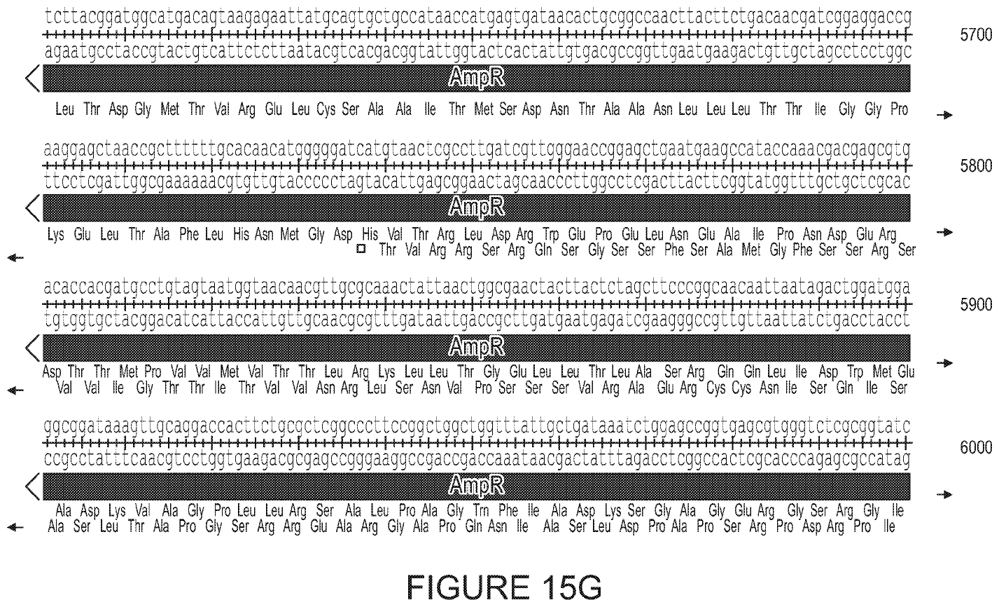

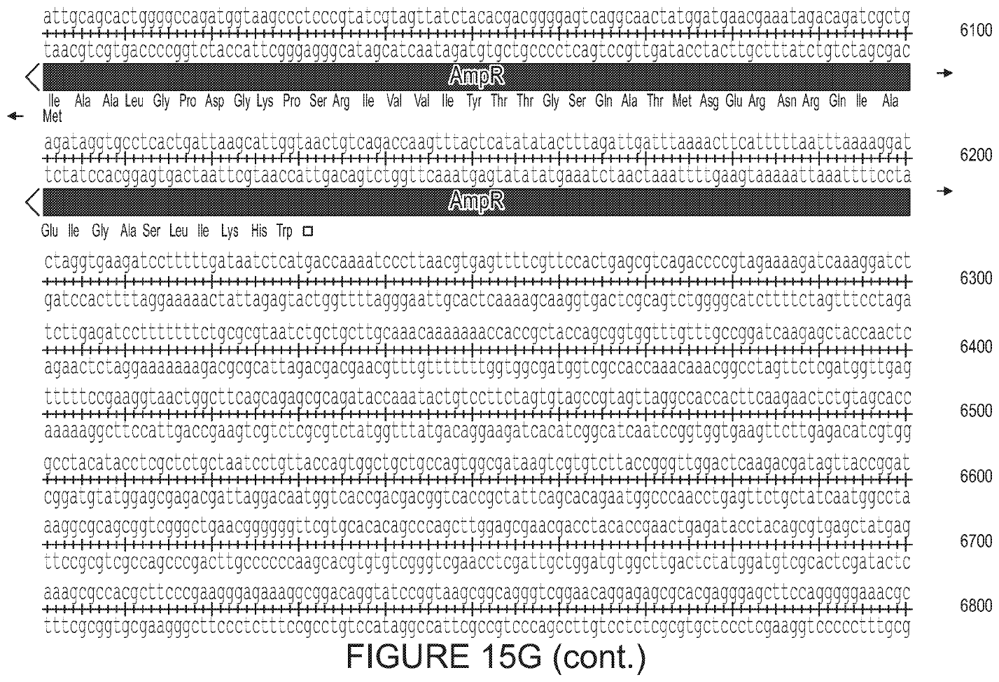

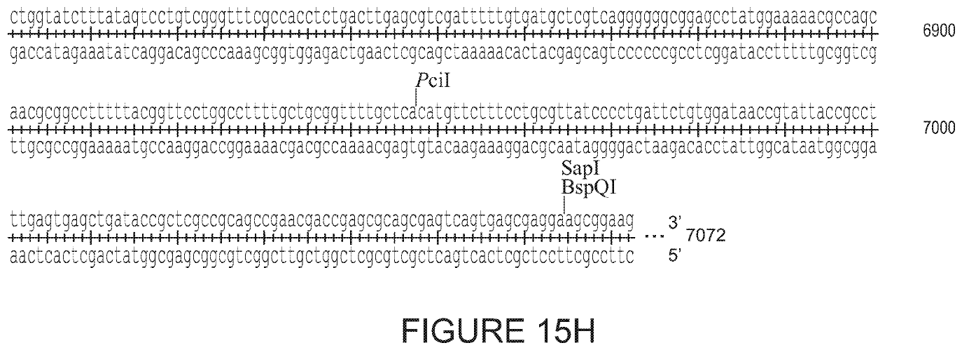

In another embodiment, the RedO promoter comprises nucleotides 457-2514 of the nucleotide sequence depicted in FIG. 15 (SEQ ID NO: 49), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 457-2514 of the nucleotide sequence depicted in FIG. 15 (SEQ ID NO: 49).

In certain embodiments, a suitable PR-specific promoter is a guanine nucleotide-binding protein G subunit alpha-2 (GNAT2) promoter.

As used interchangeably herein, the terms “GNAT2” and “guanine nucleotide-binding protein G subunit alpha-2 (GNAT2) promoter” also known as G Protein Subunit Alpha Transducin 2, also known as Guanine Nucleotide Binding Protein (G Protein), Alpha Transducing Activity Polypeptide 2, Guanine Nucleotide-Binding Protein G(T) Subunit Alpha-2, Transducin Alpha-2 Chain, GNATC, Transducin, Cone-Specific, Alpha Polypeptide, Cone-Type Transducin Alpha Subunit, and ACHM4, refers to the well-known G protein that stimulates the coupling of rhodopsin and cGMP-phoshodiesterase during visual impulses. The nucleotide sequence of the genomic region containing the human GNAT2 gene (including the region upstream of the coding region of human GNAT2 gene which includes the GNAT2 promoter region) is also known and may be found in, for example, GenBank Reference Sequence NC_000001.11 (SEQ ID NO: 9, the entire contents of which is incorporated herein by reference).

In some embodiments, suitable GNAT2 promoters for use in the present invention include nucleic acid molecules which include nucleotides 4873-6872 of SEQ ID NO:9; or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 4873-6872 of SEQ ID NO:9.

In other embodiments, suitable GNAT2 promoters for use in the present invention comprise the nucleotide sequence of SEQ ID NO:17; or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of the nucleotide sequence of SEQ ID NO:17.

In one embodiment, suitable GNAT2 promoters for use in the present invention comprise the nucleotide sequence of SEQ ID NO:18; or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of the nucleotide sequence of SEQ ID NO:18.

In another embodiment, suitable GNAT2 promoters for use in the present invention comprise the nucleotide sequence of SEQ ID NO:19; or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of the nucleotide sequence of SEQ ID NO:19.

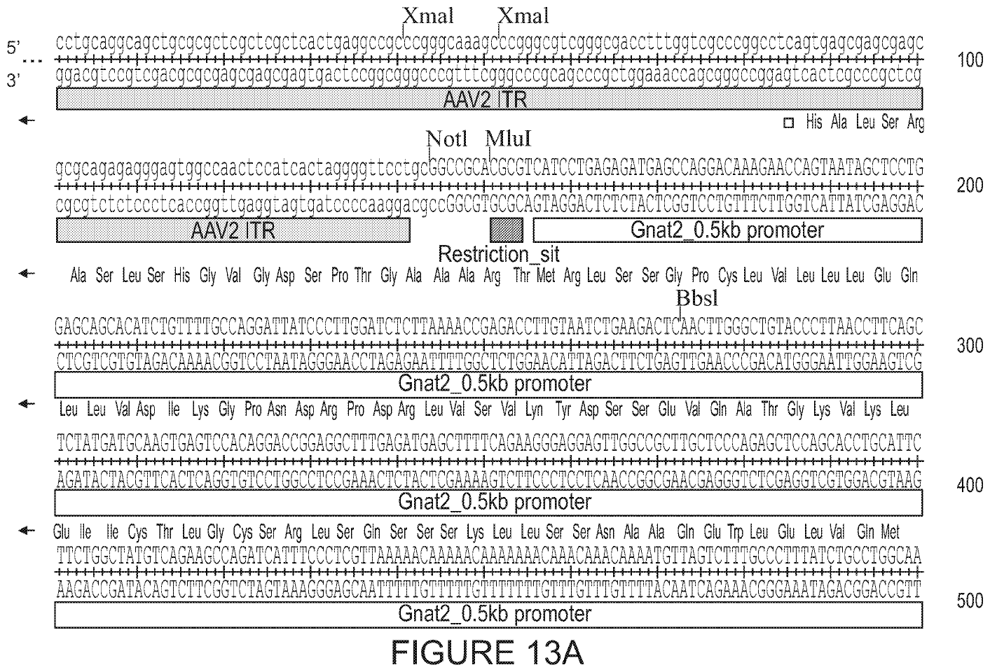

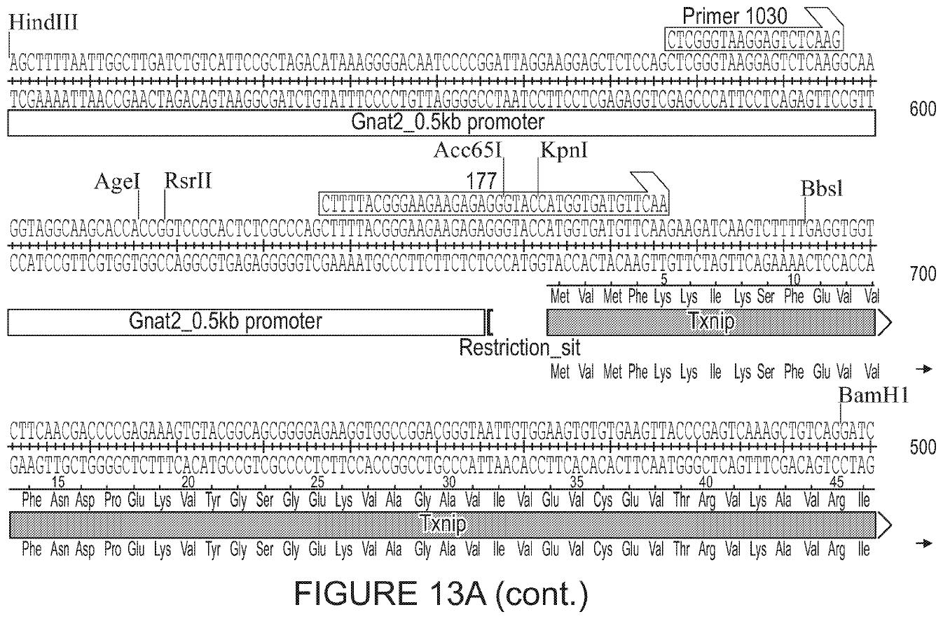

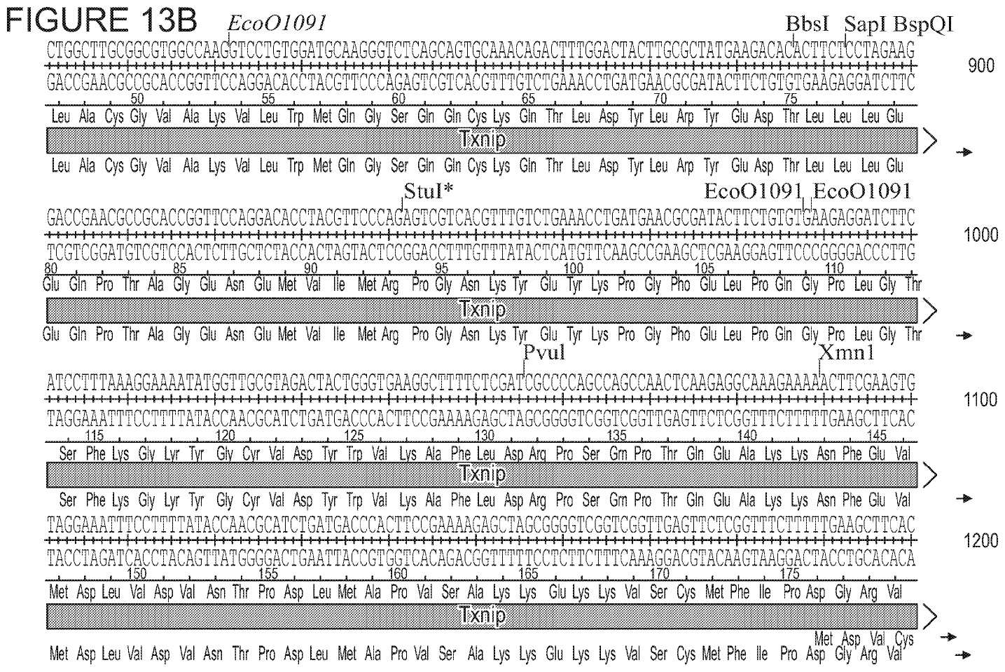

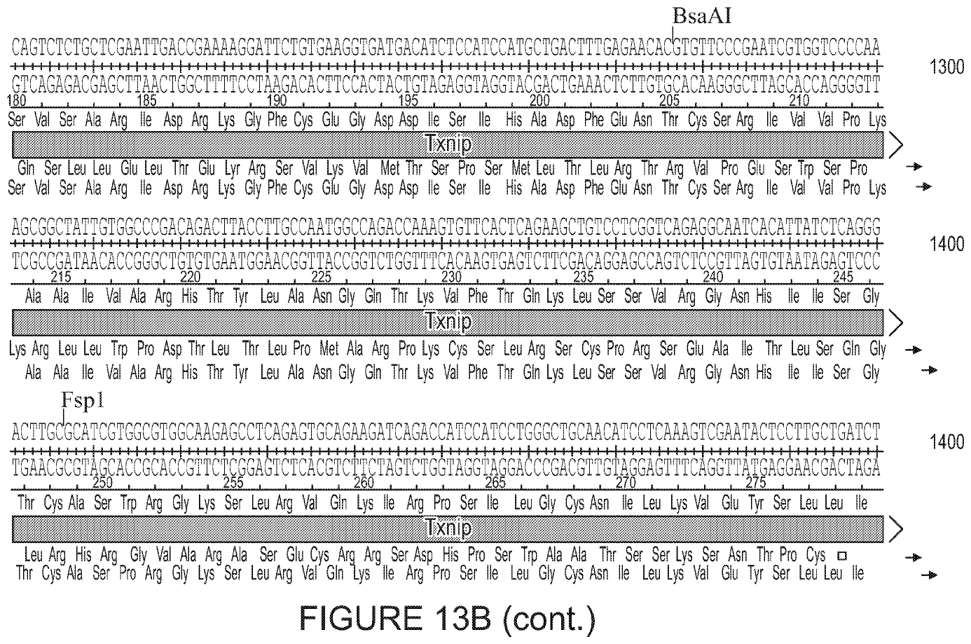

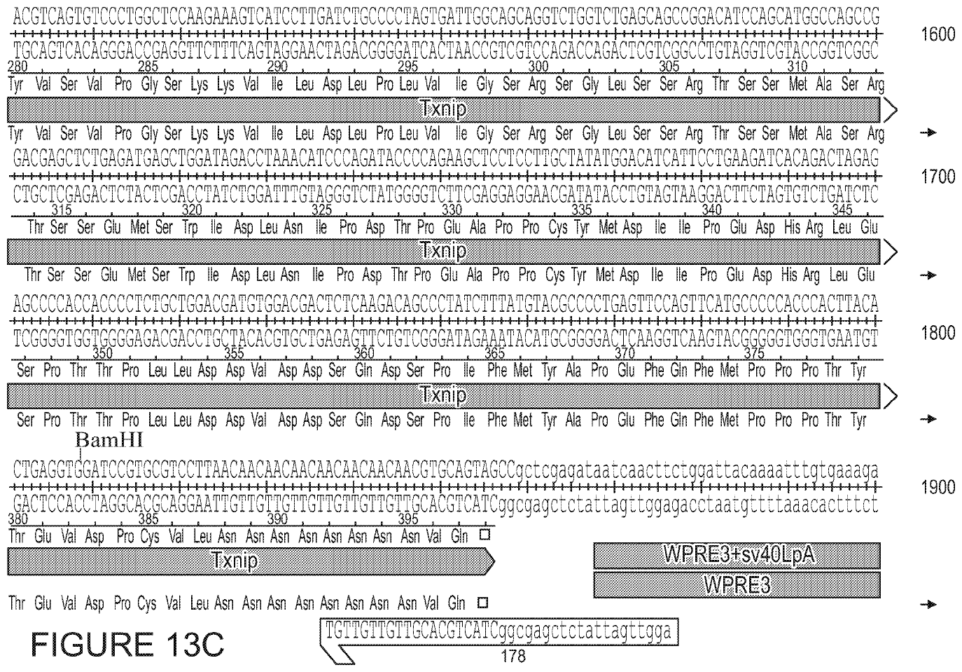

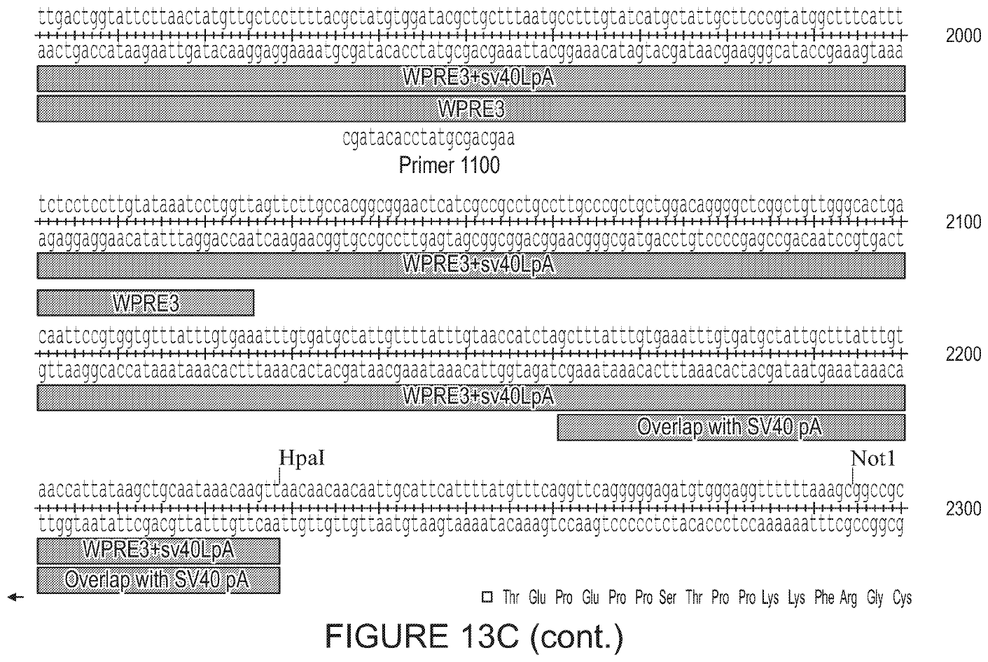

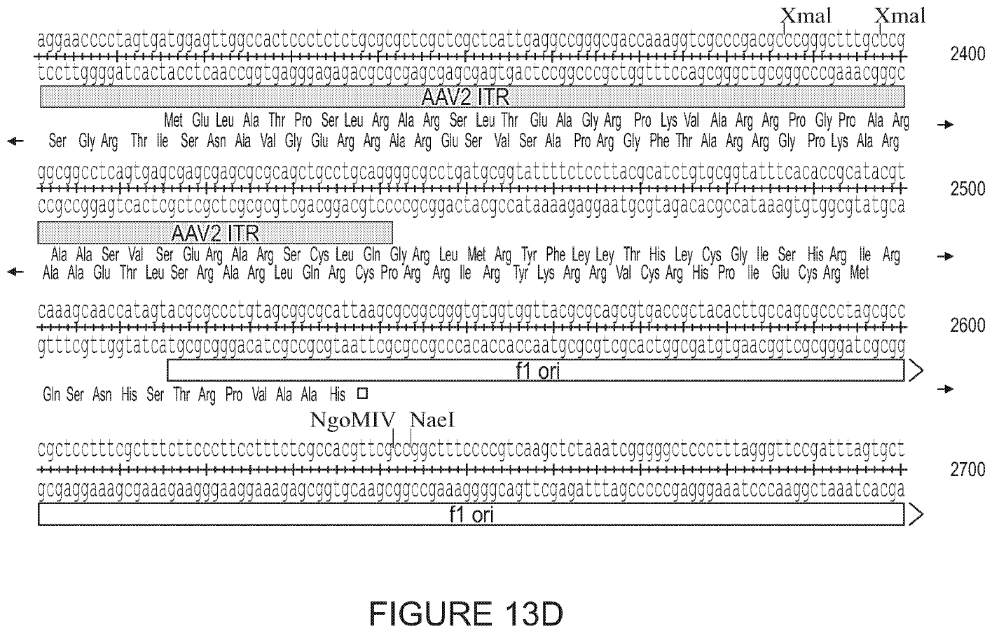

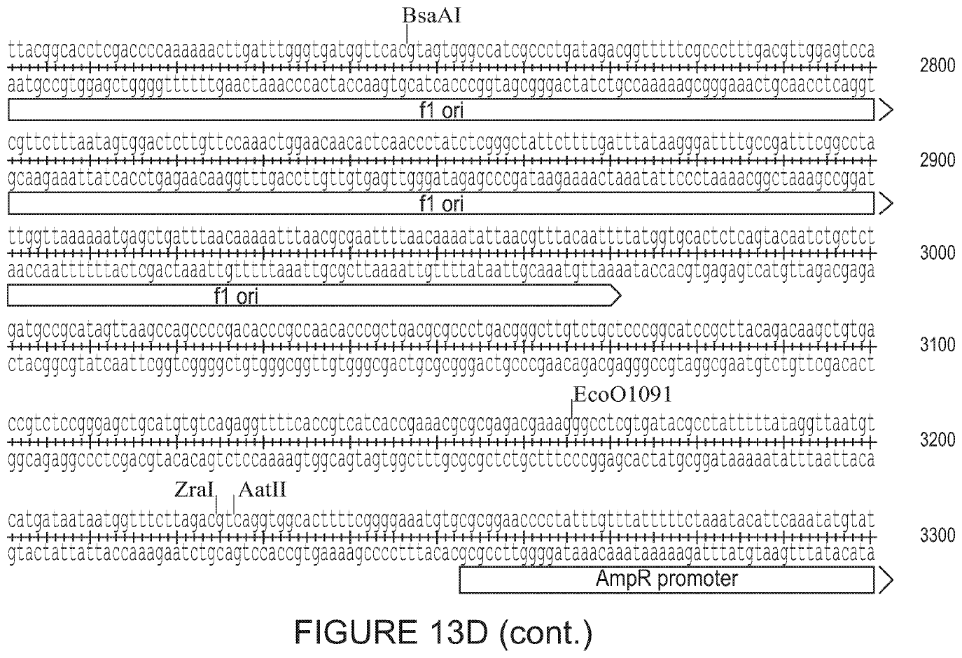

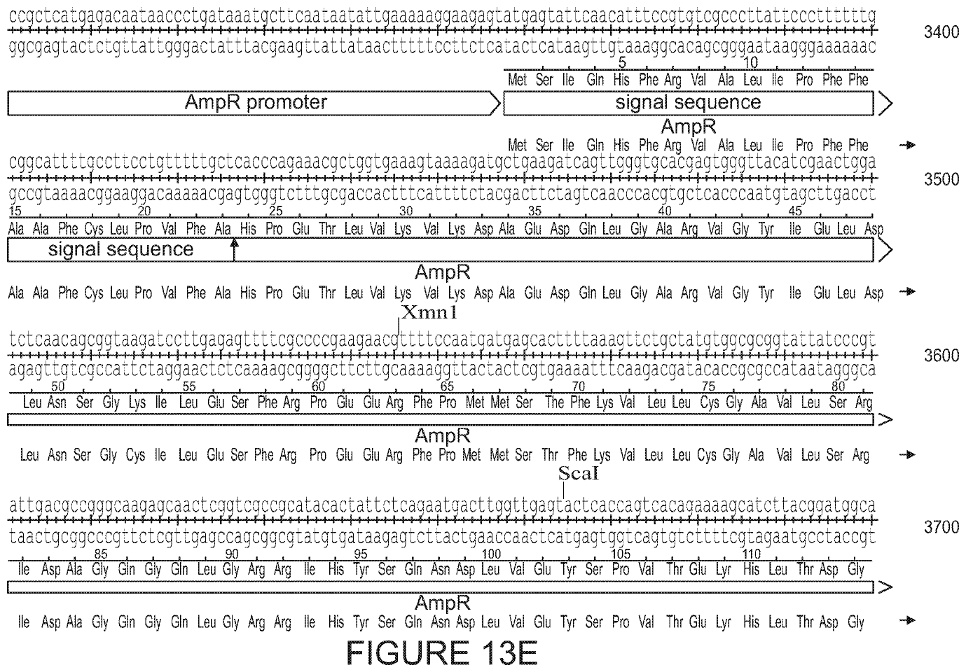

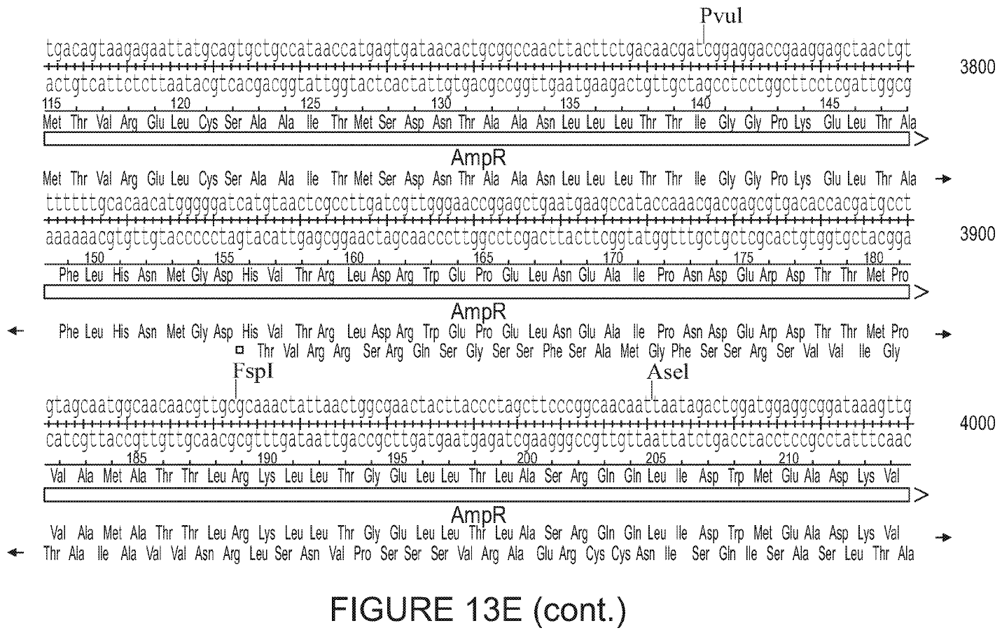

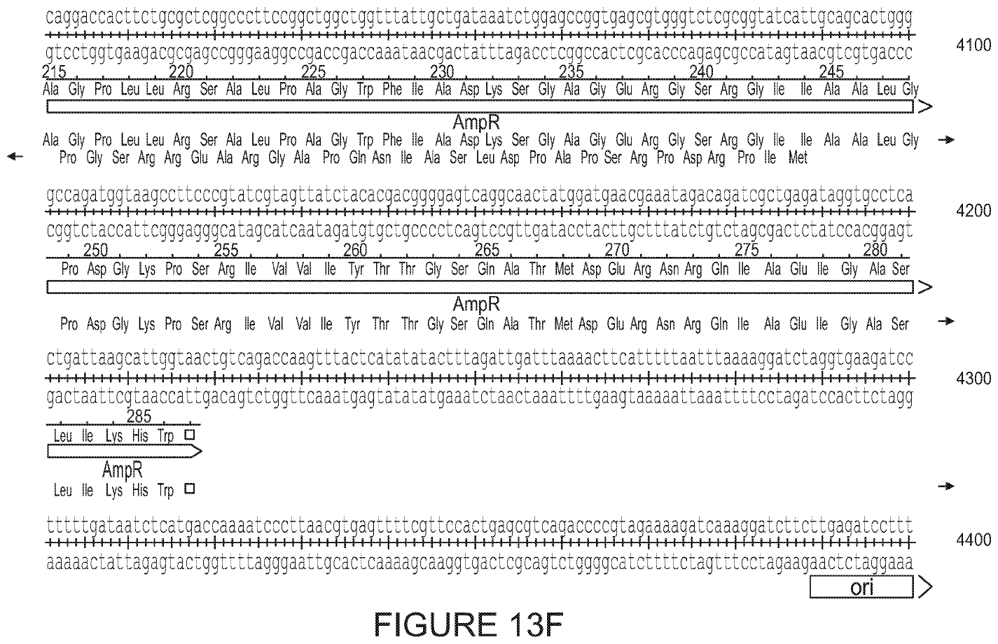

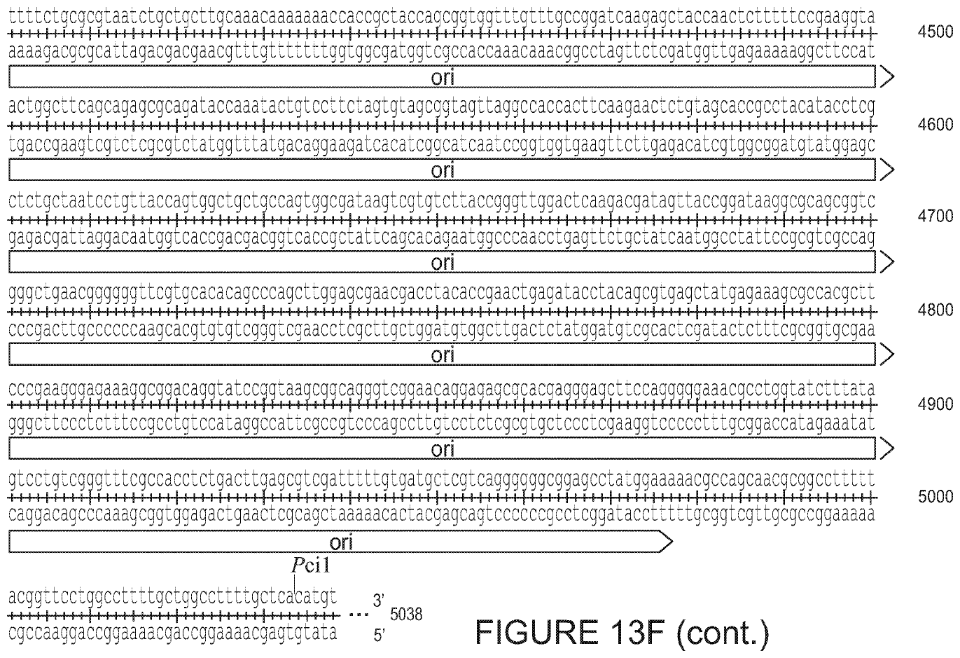

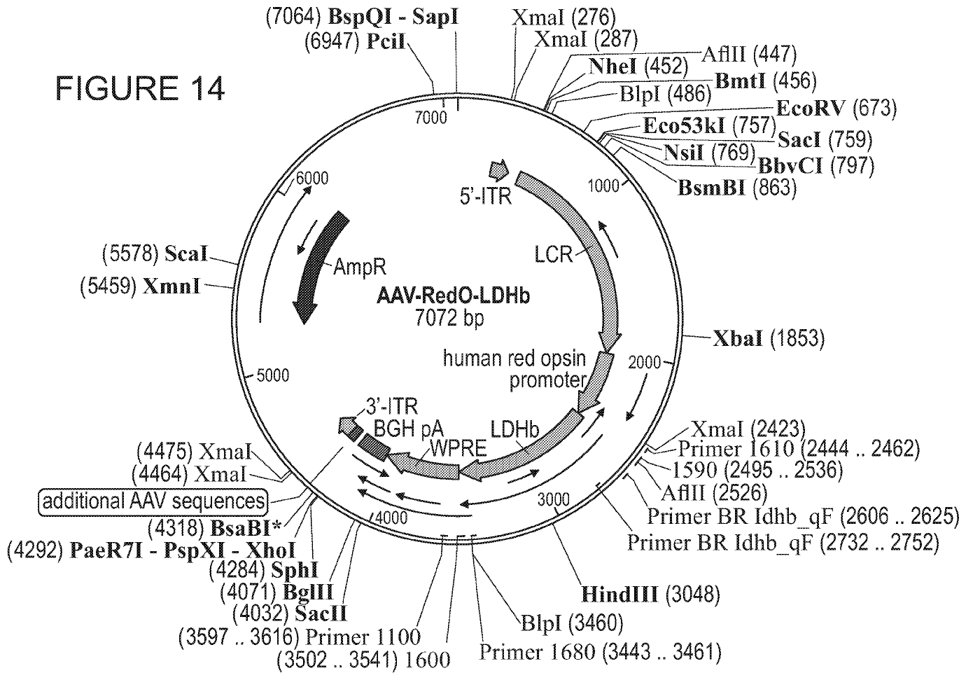

In one embodiment, the GNAT2 promoter comprises nucleotides 156-655 of the nucleotide sequence depicted in FIG. 13 (SEQ ID NO: 39), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 156-655 of the nucleotide sequence depicted in FIG. 13 (SEQ ID NO: 39).

As used herein, the term “TXNIP” refers to thioredoxin-interacting protein, a member of the alpha arrestin protein family. Thioredoxin is a thiol-oxidoreductase that is a major regulator of cellular redox signaling which protects cells from oxidative stress. TXNIP inhibits the antioxidative function of thioredoxin resulting in the accumulation of reactive oxygen species and cellular stress, and functions as a regulator of cellular metabolism and of endoplasmic reticulum (ER) stress. TXNIP is also known as Upregulated By 1,25-Dihydroxyvitamin D-3; Vitamin D3 Up-Regulated Protein 1; Thioredoxin Binding Protein 2; VDUP1; Thioredoxin-Binding Protein 2; EST01027; HHCPA78; ARRDC6; and THIF.

There are two transcript variants of human TXNIP and two transcript variants of mouse TXNIP, the nucleotide and amino acid sequences of which are known and may be found in, for example, GenBank Reference Sequences NM_006472.5, NM_001313972.1, NM_001009935.2 and NM_023719.2 (SEQ ID NOs:1-4, respectively, the entire contents of each of which are incorporated herein by reference).

In one embodiment, a nucleic acid molecule encoding TXNIP comprises nucleotides 366-1541 of SEQ ID NO:1, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 366-1541 of SEQ ID NO:1.

In another embodiment, the nucleic acid molecule encoding TXNIP comprises nucleotides 162-1172 of SEQ ID NO:2, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 162-1172 of SEQ ID NO:2.

In another embodiment, the nucleic acid molecule encoding TXNIP comprises nucleotides 280-1473 of SEQ ID NO:3, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 280-1473 of SEQ ID NO:3.

In another embodiment, the nucleic acid molecule encoding TXNIP comprises nucleotides 280-1470 of SEQ ID NO:4, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 280-1470 of SEQ ID NO:4.

In another embodiment, the nucleic acid molecule encoding TXNIP comprises nucleotides 2521-3714 of the nucleotide sequence depicted in FIG. 11 (SEQ ID NO:26), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 2521-3714 of the nucleotide sequence depicted in FIG. 11 (SEQ ID NO:26).

In another embodiment, the nucleic acid molecule encoding TXNIP comprises nucleotides 663-1856 of the nucleotide sequence depicted in FIG. 13 (SEQ ID NO: 39), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 663-1856 of the nucleotide sequence depicted in FIG. 13 (SEQ ID NO: 39).

The invention further encompasses nucleic acid molecules that differ, due to degeneracy of the genetic code, from the nucleotide sequence of nucleic acids encoding a TXNIP polypeptide, and, thus, encode the same protein.

As used herein, the term “LDHB” refers to the B subunit of the lactate dehydrogenase enzyme, which catalyzes the interconversion of pyruvate and lactate with concomitant interconversion of NADH and NAD+ in a post-glycolysis process. There are two transcript variants of human LDHB and one transcript variant of mouse LDHB, the nucleotide and amino acid sequences of which are known and may be found in, for example, GenBank Reference Sequences NM_002300.7, NM_001174097.2, and NM_008492.3 (SEQ ID NOs:5-7, respectively, the entire contents of each of which are incorporated herein by reference).

In one embodiment, a nucleic acid molecule encoding LDHB comprises nucleotides 112-1116 of SEQ ID NO:5, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 112-1116 of SEQ ID NO:5.

In another embodiment, the nucleic acid molecule encoding LDHB comprises nucleotides 334-1338 of SEQ ID NO:6, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 334-1338 of SEQ ID NO:6.

In another embodiment, the nucleic acid molecule encoding LDHB comprises nucleotides 112-1116 of SEQ ID NO:7, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 112-1116 of SEQ ID NO:7.

In another embodiment, the nucleic acid molecule encoding LDHB comprises nucleotides 2517-3521 of the nucleotide sequence depicted in FIG. 15 (SEQ ID NO: 49), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 2517-3521 of the nucleotide sequence depicted in FIG. 15 (SEQ ID NO: 49).

The invention further encompasses nucleic acid molecules that differ, due to degeneracy of the genetic code, from the nucleotide sequence of nucleic acids encoding a LDHB polypeptide, and, thus, encode the same protein.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions total # of positions (e.g., overlapping positions)×100).

The determination of percent identity between two sequences may be accomplished using a mathematical algorithm. A non-limiting example of a mathematical algorithm utilized for the comparison of two sequences is the algorithm of Karlin and Altschul (1990) Proc. Natl. Acad. Sol. USA 87:2264-2268, modified as in Karlin and Altschul (1993) Proc. Nati. Accid Sci. USA 90:5873-5877. Such an algorithm is incorporated into the BLASTN and BLASTX programs of Altschul, et al. (1990) J. Mol. Biol. 215:403-410. BLAST nucleotide searches can be performed with the BLASTN program, score=100, wordlength=12 to obtain nucleotide sequences homologous to a nucleic acid molecules of the invention. BLAST protein searches can be performed with the BLASTP program, score—50, wordlength=3 to obtain amino acid sequences homologous to a protein molecules of the invention. To obtain gapped alignments for comparison purposes, a newer version of the BLAST algorithm called Gapped BLAST can be utilized as described in Altschul et al. (1997) Nucleic Acids Res 25:3389-3402, which is able to perform gapped local alignments for the programs BLASTN, BLASTP and BLASTX.

In some embodiments, the expression cassettes of the invention further comprise an intron between the promoter and the nucleic acid molecule endoing TXNIP and/or between the promoter and the nucleic acid molecule endoing LDHB.

As used herein, “an intron” refers to a non-coding nucleic acid molecule which is removed by RNA splicing during maturation of a final RNA product.

In one embodiment, the intron is an SV40 intron, e.g., the intron comprises the nucleotide sequence of SEQ ID NO:20, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of the nucleotide sequence of SEQ ID NO: 20.

In yet another embodiment, the intron is a human beta-globin intron, e.g., the intron comprises the nucleotide sequence of SEQ ID NO:12, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of the nucleotide sequence of SEQ ID NO: 12.

In another embodiment, the intron is a chimeric intron.

A “chimeric intron” is an artificial (or non-naturally occurring intron that enhances mRNA processing and increases expression levels of a downstream open reading frame.

In some embodiments of the invention, for example, when the expression cassette comprises a PR-specific promoter operably linked to a nucleic acid molecule encoding TXNIP and a nucleic acid molecule encoding LDHB, i.e., TXNIP and LDHB are co-expressed by the PR-specific promoter, the expression cassette further comprises a linker between the nucleic acid molecule encoding TXNIP and the nucleic acid molecule encoding LDHB. Suitable linkers for co-expression of genes from a single promoter are known in the art.

In one embodiment, a suitable linker comprises a nucleotide sequence encoding a 2A peptide. As used herein, a “2A peptide” refers to the art-known peptides also referred to as “self-cleaving 2A peptides” first discovered in picornaviruses. 2A peptides are short (about 20 amino acids) and produce equimolar levels of multiple genes from the same mRNA. Exemplary nucleotide sequences of suitable 2A peptides are provided in SEQ ID NOs:21-24.

In some embodiments, the expression cassettes of the invention further comprise a post-transcriptional regulatory region.

The term “post-transcriptional regulatory region”, as used herein, refers to any polynucleotide that facilitates the expression, stabilization, or localization of the sequences contained in the cassette or the resulting gene product.

In one embodiment, a post-transcriptional regulatory region suitable for use in the expression cassettes of the invention includes a Woodchuck hepatitis virus post-transcriptional regulatory element.

As used herein, the term “Woodchuck hepatitis virus posttranscriptional regulatory element” or “WPRE,” refers to a DNA sequence that, when transcribed, creates a tertiary structure capable of enhancing the expression of a gene. See Lee Y, et al, Exp. Physiol. 2005; 90(1):33-37 and Donello J, et al, J. Virol. 1998; 72(6):5085-5092.

In one embodiment, a WPRE includes the nucleotide sequence of SEQ ID NO: 10 (See, e.g., J Virol. 1998 June; 72(6): 5085-5092), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of the nucleotide sequence of SEQ ID NO: 10.

In another embodiment, a WPRE includes the nucleotide sequence of SEQ ID NO: 11, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of the nucleotide sequence of SEQ ID NO: 11.

In another embodiment, a WPRE includes nucleotides 3722-4263 of the nucleotide sequence depicted in FIG. 11 (SEQ ID NO:26), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 3722-4263 of the nucleotide sequence depicted in FIG. 11 (SEQ ID NO:26).

In another embodiment, a WPRE includes nucleotides 1868-2025 of the nucleotide sequence depicted in FIG. 13 (SEQ ID NO: 39), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 1868-2025 of the nucleotide sequence depicted in FIG. 13 (SEQ ID NO: 39).

In another embodiment, a WPRE includes nucleotides 3529-4070 of the nucleotide sequence depicted in FIG. 15 (SEQ ID NO: 49), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 3529-4070 of the nucleotide sequence depicted in FIG. 15 (SEQ ID NO: 49).

In some embodiments, the expression cassettes of the invention further comprises a polyadenylation signal.

As used herein, a “polyadenylation signal” or “polyA signal,” as used herein refers to a nucleotide sequence that terminates transcription. Suitable polyadenylation signals for use in the AAV vectors of the invention are known in the art and include, for example, a bovine growth hormone polyA signal (BGH pA) or an SV40 polyadenylation signal (SV40 polyA).

In one embodiment, a SV40 pA includes the nucleotide sequence of SEQ ID NO: 13, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of the nucleotide sequence of SEQ ID NO: 13.

In one embodiment, a BGH pA includes the nucleotide sequence of SEQ ID NO: 25, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of the nucleotide sequence of SEQ ID NO: 25.

In one embodiment, a BGH pA includes the nucleotide sequence of nucleotides 4270-4484 of the nucleotide sequence in FIG. 11 (SEQ ID NO:26), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 4270-4484 of the nucleotide sequence in FIG. 11 (SEQ ID NO:26).

In one embodiment, a SV40 pA includes the nucleotide sequence of nucleotides 2026-2228 of the nucleotide sequence in FIG. 13 (SEQ ID NO: 39), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 2026-2228 of the nucleotide sequence in FIG. 13 (SEQ ID NO: 39).

In one embodiment, a BGH pA includes the nucleotide sequence of nucleotides 4077-4291 of the nucleotide sequence in FIG. 15 (SEQ ID NO: 49), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 4077-4291 of the nucleotide sequence in FIG. 15 (SEQ ID NO: 49).

In some embodiments, the expression cassettes of the invention further comprise an enhancer.

The term “enhancer”, as used herein, refers to a DNA sequence element to which transcription factors bind to increase gene transcription.

The AAV vectors of the invention may also include cis-acting 5′ and 3′ inverted terminal repeat (ITR) sequences. In some embodiments, the ITR sequences are about 145 bp in length. In some embodiments, substantially the entire sequences encoding the ITRs are used in the molecule. In other embodiments, the ITRs include modifications. Procedures for modifying these ITR sequences are known in the art. See Brown T, “Gene Cloning” (Chapman & Hall, London, G B, 1995), Watson R, et al, “Recombinant DNA”, 2nd Ed. (Scientific American Books, New York, N.Y., US, 1992), Alberts B, et al, “Molecular Biology of the Cell” (Garland Publishing Inc., New York, N.Y., US, 2008), Innis M, et al, Eds., “PCR Protocols. A Guide to Methods and Applications” (Academic Press Inc., San Diego, Calif., US, 1990), Erlich H, Ed., “PCR Technology. Principles and Applications for DNA Amplification” (Stockton Press, New York, N.Y., US, 1989), Sambrook J, et al, “Molecular Cloning. A Laboratory Manual” (Cold Spring Harbor Laboratory Press, Cold Spring Harbor, N.Y., US, 1989), Bishop T, et al, “Nucleic Acid and Protein Sequence. A Practical Approach” (IRL Press, Oxford, G B, 1987), Reznikoff W, Ed., “Maximizing Gene Expression” (Butterworths Publishers, Stoneham, Mass., US, 1987), Davis L, et al, “Basic Methods in Molecular Biology” (Elsevier Science Publishing Co., New York, N.Y., US, 1986), and Schleef M, Ed., “Plasmid for Therapy and Vaccination” (Wiley-VCH Verlag GmbH, Weinheim, D E, 2001).

The AAV vectors of the invention may include ITR nucleotide sequences derived from any one of the AAV serotypes. In a preferred embodiment, the AAV vector comprises 5′ and 3′ AAV ITRs. In one embodiment, the 5′ and 3′ AAV ITRs derive from AAV2. AAV ITRs for use in the AAV vectors of the invention need not have a wild-type nucleotide sequence (See Kotin, Hum. Gene Ther., 1994, 5:793-801). As long as ITR sequences function as intended for the rescue, replication and packaging of the AAV virion, the ITRs may be altered by the insertion, deletion or substitution of nucleotides or the ITRs may be derived from any of several AAV serotypes or its mutations.

In one embodiment, a 5′ ITR includes nucleotides 248-377 of the nucleotide sequence in FIG. 11 (SEQ ID NO:26); nucleotides 1-141 of the nucleotide sequence in FIG. 13 (SEQ ID NO: 39); or nucleotides 248-377 of the nucleotide sequence in FIG. 15 (SEQ ID NO: 49), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 248-377 of the nucleotide sequence in FIG. 11 (SEQ ID NO:26); nucleotides 1-141 of the nucleotide sequence in FIG. 13 (SEQ ID NO: 39); or nucleotides 248-377 of the nucleotide sequence in FIG. 15 (SEQ ID NO: 49).

In one embodiment, a 3′ ITR includes nucleotides 4571-4201 of the nucleotide sequence in FIG. 11 (SEQ ID NO:26); nucleotides 2301-2441 of the nucleotide sequence in FIG. 13 (SEQ ID NO: 39); or nucleotides 4378-4508 of the nucleotide sequence in FIG. 15 (SEQ ID NO: 49), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 4571-4201 of the nucleotide sequence in FIG. 11 (SEQ ID NO:26); nucleotides 2301-2441 of the nucleotide sequence in FIG. 13 (SEQ ID NO: 39); or nucleotides 4378-4508 of the nucleotide sequence in FIG. 15 (SEQ ID NO: 49).

In addition, an AAV vector can contain one or more selectable or screenable marker genes for initially isolating, identifying, or tracking host cells that contain DNA encoding the ithe AAV vector (and/or rep, cap and/helper genes), e.g., antibiotic resistance, as described herein.

As indicated above, the AAV vectors of the invention may be packaged into AAV viral particles for use in the methods, e.g., gene therapy methods, of the invention (discussed below) to produce AAV vector particles using methods known in the art.

Such methods generally include packaging the AAV vectors of the invention into infectious AAV viral particles in a host cell that has been transfected with a vector encoding and expressing rep and cap gene products (i.e. AAV Rep and Cap proteins), and with a vector which encodes and expresses a protein from the adenovirus open reading frame E4orf6.

Suitable AAV Caps may be derived from any serotype. In one embodiment, the capsid is derived from the AAV of the group consisting on AAV1, AAV2, AAV3, AAV4, AAV5, AAV6, AAV7, AAV8 and AAV9. In another embodiment, the AAV of the invention comprises a capsid derived from the AAV7m8, AAV5 or AAV8 serotypes.

In some embodiments, an AAV Cap for use in the method of the invention can be generated by mutagenesis (i.e. by insertions, deletions, or substitutions) of one of the aforementioned AAV Caps or its encoding nucleic acid. In some embodiments, the AAV Cap is at least 70%, 75%, 80%, 85%, 90%, 95%, 98%, or 99% or more similar to one or more of the aforementioned AAV Caps.

In some embodiments, the AAV Cap is chimeric, comprising domains from two, three, four, or more of the aforementioned AAV Caps. In some embodiments, the AAV Cap is a mosaic of VP1, VP2, and VP3 monomers originating from two or three different AAV or a recombinant AAV. In some embodiments, a rAAV composition comprises more than one of the aforementioned Caps.

Suitable rep may be derived from any AAV serotype. In one embodiment, the rep is derived from any of the serotypes selected from the group consisting of AAV1, AAV2, AAV3, AAV4, AAV5, AAV6, AAV7, AAV8, or AAV9. In another embodiment, the AAV rep is derived from the serotype AAV2.

Suitable helper genes may be derived from any AAV serotype and include adenovirus E4, E2a and VA.

The AAV rep, AAV cap and genes providing helper functions can be introduced into the cell by incorporating the genes into a vector such as, for example, a plasmid, and introducing the vector into a cell. The genes can be incorporated into the same plasmid or into different plasmids. In one, the AAV rep and cap genes are incorporated into one plasmid and the genes providing helper functions are incorporated into another plasmid.

The AAV vectors of the invention and the polynucleotides comprising AAV rep and cap genes and genes providing helper functions may be introduced into a host cell using any suitable method well known in the art. See Ausubel F, et al, Eds., “Short Protocols in Molecular Biology”, 4th Ed. (John Wiley and Sons, Inc., New York, N.Y., US, 1997), Brown (1995), Watson (1992), Alberts (2008), Innis (1990), Erlich (1989), Sambrook (1989), Bishop (1987), Reznikoff (1987), Davis (1986), and Schleef (2001), supra. Examples of transfection methods include, but are not limited to, co-precipitation with calcium phosphate, DEAE-dextran, polybrene, electroporation, microinjection, liposome-mediated fusion, lipofection, retrovirus infection and biolistic transfection. When the cell lacks the expression of any of the AAV rep and cap genes and genes providing adenoviral helper functions, said genes can be introduced into the cell simultaneously with the AAV vector. Alternatively, the genes can be introduced in the cell before or after the introduction of the AAV vector of the invention.

Methods of culturing packaging cells and exemplary conditions which promote the release of AAV vector particles, such as the producing of a cell lysate, are known in the art. Producer cells are grown for a suitable period of time in order to promote release of viral vectors into the media. Generally, cells may be grown for about 24 hours, about 36 hours, about 48 hours, about 72 hours, about 4 days, about 5 days, about 6 days, about 7 days, about 8 days, about 9 days, up to about 10 days. After about 10 days (or sooner, depending on the culture conditions and the particular producer cell used), the level of production generally decreases significantly. Generally, time of culture is measured from the point of viral production. For example, in the case of AAV, viral production generally begins upon supplying helper virus function in an appropriate producer cell as described herein. Generally, cells are harvested about 48 to about 100, preferably about 48 to about 96, preferably about 72 to about 96, preferably about 68 to about 72 hours after helper virus infection (or after viral production begins).

The AAV vector particles of the invention can be obtained from both: i) the cells transfected with the foregoing and ii) the culture medium of the cells after a period of time post-transfection, preferably 72 hours. Any method for the purification of the AAV vector particles from the cells or the culture medium can be used for obtaining the AAV vector particles of the invention. In a particular embodiment, the AAV vector particles of the invention are purified following an optimized method based on a polyethylene glycol precipitation step and two consecutive cesium chloride (CsCl) or iodixanol density gradient ultracentrifugation. See Ayuso et al., 2014, Zolotukhin S, et al., Gene Ther. 1999; 6; 973-985. Purified AAV vector particles of the invention can be dialyzed against an appropriate formulation buffer such as PBS, filtered and stored at −80° C. Titers of viral genomes can be determined by quantitative PCR following the protocol described for the AAV2 reference standard material using linearized plasmid DNA as standard curve. See Aurnhammer C, et al., Hum Gene Ther Methods, 2012, 23, 18-28, D'Costa S, et al., Mol Ther Methods Clin Dev. 2016, 5, 16019.

In some embodiments, the methods further comprise purification steps, such as treatment of the cell lysate with benzonase, purification of the cell lysate with the use of affinity chromatography and/or ion-exchange chromotography. See Halbert C, et al, Methods Mol. Biol. 2004; 246:201-212, Nass S, et al., Mol Ther Methods Clin Dev. 2018 Jun. 15; 9: 33-46.

AAV Rep and Cap proteins and their sequences, as well as methods for isolating or generating, propagating, and purifying such AAV, and in particular, their capsids, suitable for use in producing AAV are known in the art. See Gao, 2004, supra, Russell D, et al, U.S. Pat. No. 6,156,303, Hildinger M, et al, U.S. Pat. No. 7,056,502, Gao G, et al, U.S. Pat. No. 7,198,951, Zolotukhin S, U.S. Pat. No. 7,220,577, Gao G, et al, U.S. Pat. No. 7,235,393, Gao G, et al, U.S. Pat. No. 7,282,199, Wilson J, et al, U.S. Pat. No. 7,319,002, Gao G, et al, U.S. Pat. No. 7,790,449, Gao G, et al, US 20030138772, Gao G, et al, US 20080075740, Hildinger M, et al, WO 2001/083692, Wilson J, et al, WO 2003/014367, Gao G, et al, WO 2003/042397, Gao G, et al, WO 2003/052052, Wilson J, et al, WO 2005/033321, Vandenberghe L, et al, WO 2006/110689, Vandenberghe L, et al, WO 2007/127264, and Vandenberghe L, et al, WO 2008/027084.

### III. Pharmaceutical Compositions of the Invention

In one aspect of the invention, an AAV viral particle of the invention will be in the form of a pharmaceutical composition containing a pharmaceutically acceptable carrier. As used herein “pharmaceutically acceptable carrier” refers to any substantially non-toxic carrier conventionally useable for administration of pharmaceuticals in which the isolated polypeptide of the present invention will remain stable and bioavailable. The pharmaceutically acceptable carrier must be of sufficiently high purity and of sufficiently low toxicity to render it suitable for administration to the mammal being treated. It further should maintain the stability and bioavailability of an active agent. The pharmaceutically acceptable carrier can be liquid or solid and is selected, with the planned manner of administration in mind, to provide for the desired bulk, consistency, etc., when combined with an active agent and other components of a given composition. Suitable pharmaceutically acceptable carriers include any and all solvents, dispersion media, coatings, antibacterial and antifungal agents, isotonic and absorption delaying agents, and the like that are physiologically compatible. Pharmaceutically acceptable carriers also include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the gene therapy vector, use thereof in the pharmaceutical compositions of the invention is contemplated. Supplementary active compounds can also be incorporated into the compositions.

Pharmaceutical compositions of the invention may be formulated for delivery to animals for veterinary purposes (e.g. livestock (cattle, pigs, dogs, mice, rats), and other non-human mammalian subjects, as well as to human subjects.

In a particular embodiment, the pharmaceutical compositions of the present invention are in the form of injectable compositions. The compositions can be prepared as an injectable, either as liquid solutions or suspensions. The preparation may also be emulsified. Suitable excipients are, for example, water, saline, dextrose, glycerol, ethanol, phosphate buffered saline or the like and combinations thereof. In addition, if desired, the preparation may contain minor amounts of auxiliary substances such as wetting or emulsifying agents, pH-buffering agents, adjuvants, surfactant or immunopotentiators.

In a particular embodiment, the AAV particles of the invention are incorporated in a composition suitable for intraocular administration. For example, the compositions may be designed for intravitreal, subretinal, subconjuctival, sub-tenon, periocular, retrobulbar, suprachoroidal, and/or intrascleral administration, for example, by injection, to effectively treat the retinal disorder. Additionally, a sutured or refillable dome can be placed over the administration site to prevent or to reduce “wash out”, leaching and/or diffusion of the active agent in a non-preferred direction.

Relatively high viscosity compositions, as described herein, may be used to provide effective, and preferably substantially long-lasting delivery of the nucleic acid molecules and/or vectors, for example, by injection to the posterior segment of the eye. A viscosity inducing agent can serve to maintain the nucleic acid molecules and/or vectors in a desirable suspension form, thereby preventing deposition of the composition in the bottom surface of the eye. Such compositions can be prepared as described in U.S. Pat. No. 5,292,724, the entire contents of which are hereby incorporated herein by reference.

Sterile injectable solutions can be prepared by incorporating the compositions of the invention in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation include vacuum drying and freeze-drying which yields a powder of the active ingredient plus any additional desired ingredient from a previously sterile-filtered solution thereof.

Toxicity and therapeutic efficacy of nucleic acid molecules described herein can be determined by standard pharmaceutical procedures in cell cultures or experimental animals, e.g., for determining the ED₅₀ (the dose therapeutically effective in 50% of the population). Data obtained from cell culture assays and/or animal studies can be used in formulating a range of dosage for use in humans. The dosage typically will lie within a range of concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays.

### IV. Methods of the Invention

The present invention also provides methods of use of the compositions of the invention, which generally include contacting an ocular cell with an AAV viral particle or pharmaceutical composition comprising an AAV particle of the invention.

Accordingly, in one aspect, the present invention provides methods for prolonging the viability of a photoreceptor cell, e.g., a photoreceptor cell, compromised by degenerative ocular disorder, e.g., retinitis pigmentosa, age related macular degeneration, cone rod dystrophy, and rod cone dystrophy. The methods generally include contacting the cell with an AAV viral particle or pharmaceutical composition comprising an AAV particle of the invention.

The present invention further provides methods for treating a degenerative ocular disorder in a subject having a degenerative ocular disorder, e.g., retinitis pigmentosa, age related macular degeneration, cone rod dystrophy, and rod cone dystrophy. The methods include administering to the subject a therapeutically effective amount of an AAV viral particle or pharmaceutical composition comprising an AAV particle of the invention.

The present invention also provides methods for preventing a degenerative ocular disorder in a subject having a degenerative ocular disorder, e.g., retinitis pigmentosa, age related macular degeneration, cone rod dystrophy, and rod cone dystrophy. The methods include administering to the subject a prophylactically effective amount of an AAV viral particle or pharmaceutical composition comprising an AAV particle of the invention.

In another aspect, the present invention provides methods of treating a subject having retinitis pigmentosa. The methods include administering to the subject a therapeutically effective amount of an AAV viral particle or pharmaceutical composition comprising an AAV particle of the invention.

In another aspect, the present invention provides methods of treating a subject having age-related macular degeneration. The methods include administering to the subject a therapeutically effective amount of an AAV viral particle or pharmaceutical composition comprising an AAV particle of the invention.

Generally, methods are known in the art for viral infection of the cells of interest. The virus can be placed in contact with the cell of interest or alternatively, can be injected into a subject suffering from a disorder associated with photoreceptor cell oxidative stress.

Guidance in the introduction of the compositions of the invention into subjects for therapeutic purposes are known in the art and may be obtained in the above-referenced publications, as well as in U.S. Pat. Nos. 5,631,236, 5,688,773, 5,691,177, 5,670,488, 5,529,774, 5,601,818, and PCT Publication No. WO 95/06486, the entire contents of which are incorporated herein by reference.

The compositions of the invention may be delivered to a subject by, for example, intravenous injection, local administration (see, e.g., U.S. Pat. No. 5,328,470), stereotactic injection (see, e.g., Chen et al. (1994) Proc. Natl. Acad. Sci. U.S.A. 91:3054-3057), or by in vivo electroporation (see, e.g., Matsuda and Cepko (2007) Proc. Natl. Acad. Sci. U.S.A. 104:1027-1032). Preferably, the compositions of the invention are administered to the subject locally. Local administration of the compositions described herein can be by any suitable method in the art including, for example, injection (e.g., intravitreal or subretinal, subvitreal, subconjuctival, sub-tenon, periocular, retrobulbar, suprachoroidal, and/or intrascleral injection), gene gun, by topical application of the composition in a gel, oil, or cream, by electroporation, using lipid-based transfection reagents, transcleral delivery, by implantation of scleral plugs or a drug delivery device, or by any other suitable transfection method.

Application of the methods of the invention for the treatment and/or prevention of a disorder can result in curing the disorder, decreasing at least one symptom associated with the disorder, either in the long term or short term or simply a transient beneficial effect to the subject.

Accordingly, as used herein, the terms “treat,” “treatment” and “treating” include the application or administration of compositions, as described herein, to a subject who is suffering from a degenerative ocular disease or disorder, or who is susceptible to such conditions with the purpose of curing, healing, alleviating, relieving, altering, remedying, ameliorating, improving or affecting such conditions or at least one symptom of such conditions. As used herein, the condition is also “treated” if recurrence of the condition is reduced, slowed, delayed or prevented.

The term “prophylactic” or “therapeutic” treatment refers to administration to the subject of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if administered after manifestation of the unwanted condition, the treatment is therapeutic (i.e., it is intended to diminish, ameliorate or maintain the existing unwanted condition or side effects therefrom).

“Therapeutically effective amount,” as used herein, is intended to include the amount of a composition of the invention that, when administered to a patient for treating a degenerative ocular disease, is sufficient to effect treatment of the disease (e.g., by diminishing, ameliorating or maintaining the existing disease or one or more symptoms of disease). The “therapeutically effective amount” may vary depending on the composition, how the composition is administered, the disease and its severity and the history, age, weight, family history, genetic makeup, stage of pathological processes mediated by the disease expression, the types of preceding or concomitant treatments, if any, and other individual characteristics of the patient to be treated.

“Prophylactically effective amount,” as used herein, is intended to include the amount of a composition that, when administered to a subject who does not yet experience or display symptoms of e.g., a degenerative ocular disorder, but who may be predisposed to the disease, is sufficient to prevent or ameliorate the disease or one or more symptoms of the disease. Ameliorating the disease includes slowing the course of the disease or reducing the severity of later-developing disease. The “prophylactically effective amount” may vary depending on the composition, how the composition is administered, the degree of risk of disease, and the history, age, weight, family history, genetic makeup, the types of preceding or concomitant treatments, if any, and other individual characteristics of the patient to be treated.

A “therapeutically-effective amount” or “prophylacticaly effective amount” also includes an amount of a composition that produces some desired local or systemic effect at a reasonable benefit/risk ratio applicable to any treatment. A composition employed in the methods of the present invention may be administered in a sufficient amount to produce a reasonable benefit/risk ratio applicable to such treatment.

Subjects suitable for treatment using the regimens of the present invention should have or are susceptible to developing a degenerative ocular disease or disorder. For example, subjects may be genetically predisposed to development of the disorders. Alternatively, abnormal progression of the following factors including, but not limited to visual acuity, the rate of death of cone and/or rod cells, night vision, peripheral vision, attenuation of the retinal vessels, and other ophthalmoscopic factors associated with degenerative ocular disorders such as retinitis pigmentosa may indicate the existence of or a predisposition to a retinal disorder.

In one embodiment, the disorder includes, but not limited to, retinitis pigmentosa, age related macular degeneration, cone rod dystrophy, and rod cone dystrophy. In other embodiments, the disorder is not associated with blood vessel leakage and/or growth. In certain embodiments, the disorder is not associated with diabetes. In another embodiment, the disorder is not diabetic retinopathy. In further embodiments, the disorder is not NARP (neuropathy, ataxia and retinitis pigmentosa). In one embodiment, the disorder is a disorder associated with decreased viability of cone and/or rod cells. In yet another embodiment, the disorder is a genetic disorder.

The compositions, as described herein, may be administered as necessary to achieve the desired effect and depend on a variety of factors including, but not limited to, the severity of the condition, age and history of the subject and the nature of the composition, for example, the identity of the genes or the affected biochemical pathway.

The pharmaceutical compositions of the invention may be administered in a single dose or, in particular embodiments of the invention, multiples doses (e.g. two, three, four, or more administrations) may be employed to achieve a therapeutic effect.

The therapeutic or preventative regimens may cover a period of at least about 2, 3, 4, 5, 6, 7, 8, 9, 10, 11, 12, 13, 14, 15, 16, 17, 18, 19, 20, 21, 22, 23 or 24 weeks, or be chronically administered to the subject.

In one embodiment, the viability or survival of photoreceptor cells, such as cones cells, is, e.g., about 1 week, about 2 weeks, about 3 weeks, about 4 weeks, about 5 weeks, about 6 weeks, about 7 weeks, about 8 weeks, about 3 years, about 4 years, about 5 years, about 10 years, about 15, years, about 20 years, about 25 years, about 30 years, about 40 years, about 50 years, about 60 years, about 70 years, and about 80 years.

In general, the nucleic acid molecules and/or the vectors of the invention are provided in a therapeutically effective amount to elicit the desired effect, e.g., increase Nrf2 expression. The quantity of the viral particle to be administered, both according to number of treatments and amount, will also depend on factors such as the clinical status, age, previous treatments, the general health and/or age of the subject, other diseases present, and the severity of the disorder. Precise amounts of active ingredient required to be administered depend on the judgment of the gene therapist and will be particular to each individual patient. Moreover, treatment of a subject with a therapeutically effective amount of the nucleic acid molecules and/or the vectors of the invention can include a single treatment or, preferably, can include a series of treatments. It will also be appreciated that the effective dosage used for treatment may increase or decrease over the course of a particular treatment. Changes in dosage may result from the results of diagnostic assays as described herein. The pharmaceutical compositions can be included in a container, pack, or dispenser together with instructions for administration.

In some embodiments, a therapeutically effective amount or a prophylactically effective amount of a viral particle of the invention (or pharmaceutical composition of the invention) is in titers ranging from about 1×10⁵, about 1.5×10⁵, about 2×10⁵, about 2.5×10⁵, about 3×10⁵, about 3.5×10⁵, about 4×10⁵, about 4.5×10⁵, about 5×10⁵, about 5.5×10⁵, about 6×10⁵, about 6.5×10⁵, about 7×10⁵, about 7.5×10⁵, about 8×10⁵, about 8.5×10⁵, about 9×10⁵, about 9.5×10⁵, about 1×10⁶, about 1.5×10⁶, about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10, about 9×10⁶, about 9.5×10⁶, about 1×10⁷, about 1.5×10⁷, about 2×10⁷, about 2.5×10⁷, about 3×10⁷, about 3.5×10⁷, about 4×10⁷, about 4.5×10⁷, about 5×10⁷, about 5.5×10⁷, about 6×10⁷, about 6.5×10⁷, about 7×10⁷, about 7.5×10⁷, about 8×10⁷, about 8.5×10⁷, about 9×10⁷, about 9.5×10⁷, about 1×10⁸, about 1.5×10⁸, about 2×10⁸, about 2.5×10⁸, about 3×10⁸, about 3.5×10⁸, about 4×10⁸, about 4.5×10⁸, about 5×10⁸, about 5.5×10⁸, about 6×10⁸, about 6.5×10⁸, about 7×10⁸, about 7.5×10⁸, about 8×10⁸, about 8.5×10⁸, about 9×10⁸, about 9.5×10⁸, about 1×10⁹, about 1.5×10⁹, about 2×10⁹, about 2.5×109⁸, about 3×10⁹, about 3.5×10⁹, about 4×10⁹, about 4.5×10⁹, about 5×10⁹, about 5.5×10⁹, about 6×10⁹, about 6.5×10⁹, about 7×10⁹, about 7.5×10⁹, about 8×10⁹, about 8.5×10⁹, about 9×10⁹, about 9.5×10⁹, about 1×10¹⁰, about 1.5×10¹⁰, about 2×10¹⁰, about 2.5×10¹⁰, about 3×10¹⁰, about 3.5×10¹⁰, about 4×10¹⁰, about 4.5×10¹⁰, about 5×10¹⁰, about 5.5×10¹⁰ about 6×10¹⁰ about 6.5×10¹⁰, about 7×10¹⁰, about 7.5×10¹⁰, about 8×10¹⁰, about 8.5×10¹⁰ about 9×10¹⁰ about 9.5×10¹⁰, about 1×10¹¹, about 1.5×10¹¹, about 2×10¹¹, about 2.5×10¹¹, about 3×10¹¹, about 3.5×10¹¹, about 4×10¹¹, about 4.5×10¹¹, about 5×10¹¹, about 5.5×10¹¹, about 6×10¹¹, about 6.5×10¹¹, about 7×10¹¹, about 7.5×10¹¹, about 8×10¹¹, about 8.5×10¹¹, about 9×10¹¹, about 9.5×10¹¹, about 1×10¹² viral particles (vp).

In some embodiments, a therapeutically effective amount or a prophylactically effective amount of a viral particle of the invention (or pharmaceutical composition of the invention) is in genome copies (“GC”), also referred to as “viral genomes” (“vg”) ranging from about 1×10⁵, about 1.5×10⁵, about 2×10⁵, about 2.5×10⁵, about 3×10⁵, about 3.5×10⁵, about 4×10⁵, about 4.5×10⁵, about 5×10⁵, about 5.5×10⁵, about 6×10⁵, about 6.5×10⁵, about 7×10⁵, about 7.5×10⁵, about 8×10⁵, about 8.5×10⁵, about 9×10⁵, about 9.5×10⁵, about 1×10⁶, about 1.5×10⁶, about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10, about 9×10⁶, about 9.5×10⁶, about 1×10⁷, about 1.5×10⁷, about 2×10⁷, about 2.5×10⁷, about 3×10⁷, about 3.5×10⁷, about 4×10⁷, about 4.5×10⁷, about 5×10⁷, about 5.5×10⁷, about 6×10⁷, about 6.5×10⁷, about 7×10⁷, about 7.5×10⁷, about 8×10⁷, about 8.5×10⁷, about 9×10⁷, about 9.5×10⁷, about 1×10⁸, about 1.5×10⁸, about 2×10⁸, about 2.5×10⁸, about 3×10⁸, about 3.5×10⁸, about 4×10⁸, about 4.5×10⁸, about 5×10⁸, about 5.5×10⁸, about 6×10⁸, about 6.5×10⁸, about 7×10⁸, about 7.5×10⁸, about 8×10⁸, about 8.5×10⁸, about 9×10⁸, about 9.5×10⁸, about 1×10⁹, about 1.5×10⁹, about 2×10⁹, about 2.5×109⁸, about 3×10⁹, about 3.5×10⁹, about 4×10⁹, about 4.5×10⁹, about 5×10⁹, about 5.5×10⁹, about 6×10⁹, about 6.5×10⁹, about 7×10⁹, about 7.5×10⁹, about 8×10⁹, about 8.5×10⁹, about 9×10⁹, about 9.5×10⁹, about 1×10¹⁰, about 1.5×10¹⁰, about 2×10¹⁰, about 2.5×10¹⁰, about 3×10¹⁰, about 3.5×10¹⁰, about 4×10¹⁰, about 4.5×10¹⁰, about 5×10¹⁰, about 5.5×10¹⁰, about 6×10¹⁰, about 6.5×10¹⁰, about 7×10¹⁰, about 7.5×10¹⁰, about 8×10¹⁰, about 8.5×10¹⁰, about 9×10¹⁰, about 9.5×10¹⁰, about 1×10¹¹, about 1.5×10¹¹, about 2×10¹¹, about 2.5×10¹¹, about 3×10¹¹, about 3.5×10¹¹, about 4×10¹¹, about 4.5×10¹¹, about 5×10¹¹, about 5.5×10¹¹, about 6×10¹¹, about 6.5×10¹¹, about 7×10¹¹, about 7.5×10¹¹, about 8×10¹¹, about 8.5×10¹¹, about 9×10¹¹, about 9.5×10¹¹, about 1×10¹² vg.

Any method known in the art can be used to determine the genome copy (GC) number of the viral compositions of the invention. One method for performing AAV GC number titration is as follows: purified AAV viral particle samples are first treated with DNase to eliminate un-encapsidated AAV genome DNA or contaminating plasmid DNA from the production process. The DNase resistant particles are then subjected to heat treatment to release the genome from the capsid. The released genomes are then quantitated by real-time PCR using primer/probe sets targeting specific region of the viral genome.

In various embodiments, the methods of the present invention further comprise monitoring the effectiveness of treatment. For example, visual acuity, the rate of death of cone and/or rod cells, night vision, peripheral vision, attenuation of the retinal vessels, and other ophthalmoscopic changes associated with retinal disorders such as retinitis pigmentosa may be monitored to assess the effectiveness of treatment. Additionally, the rate of death of cells associated with the particular disorder that is the subject of treatment and/or prevention, may be monitored. Alternatively, the viability of such cells may be monitored, for example, as measured by phospholipid production. The assays described in the Examples section below may also be used to monitor the effectiveness of treatment (e.g., electroretinography—ERG).

In certain embodiments of the invention, a composition of the invention is administered in combination with an additional therapeutic agent or treatment. The compositions and an additional therapeutic agent can be administered in combination in the same composition or the additional therapeutic agent can be administered as part of a separate composition or by another method described herein.

Examples of additional therapeutic agents suitable for use in the methods of the invention include those agents known to treat retinal disorders, such as retinitis pigmentosa and age-related macular degeneration and include, for example, fat soluble vitamins (e.g., vitamin A, vitamin E, and ascorbic acid), calcium channel blockers (e.g., diltiazem) carbonic anhydrase inhibitors (e.g., acetazolamide and methazolamide), anti-angiogenics (e.g., antiVEGF antibodies), growth factors (e.g., rod-derived cone viability factor (RdCVF), BDNF, CNTF, bFGF, and PEDF), antioxidants, other gene therapy agents (e.g., optogenetic gene therapy, e.g., channelrhodopsin, melanopsin, and halorhodopsin), and compounds that drive photoreceptor regeneration by, e.g., reprogramming Müller cells into photoreceptor progenitors (e.g., alpha-aminoadipate). Exemplary treatments for use in combination with the treatment methods of the present invention include, for example, retinal and/or retinal pigmented epithelium transplantation, stem cell therapies, retinal prostheses, laser photocoagulation, photodynamic therapy, low vision aid implantation, submacular surgery, and retinal translocation.

This invention is further illustrated by the following examples which should not be construed as limiting. The contents of all references, patents and published patent applications cited throughout this application, as well as the Figures, are hereby incorporated by reference.

### EXAMPLES

The following Materials and Methods were used in the Examples below.

### Animals, AAV, Neonatal Mice Subretinal Injection & Retina Histology.

AAV plasmids were prepared with existing AAV backbones using Gibson Assembly. cDNAs of inserted genes were purchased from GeneCopoeia (e.g. mTxnip), or acquired from Addgene (e.g. hHK1). AAV production and purification were done as previously described (Xiong W, et al. (2015) J Clin Invest 125(4):1433-1445). Briefly, pAAVs were used to transfect 293T cells, the cell culture medium was collected 72 hours after the transfection, and viral particles were purified using iodixanol gradients. Typically, the titer of the purificed AAVs was about 1E9 vg/μL.

For delivery of AAV to retinitis pigmentosa (RP) mice, about 6E8 to 1E9 vg/eye of AAV (including AAV8-RedO-Txnip) were subretinally injected into P0 rd1 or rd10 mouse eyes. A 3e8 vg/eye of AAV8-RedO-H2BGFP was co-injected with the Txnip AAV to label the cone nuclei for quantification.

For rd1 cone histology, animals were euthanized at P50, and the eyes were harvested and flat-mounted on glass coverslips for Keyence microscope imaging. The GFP-positive cones within the ½ radius of the retina were counted using an automated MATLAB program without human bias. The numbers of cones in +Txnip RP retina were compared to control RP retina that are only injected with RedO-H2BGFP for statistical analysis.

For rd10 mice, the injected eyes were in vivo imaged with a fluorescence fundus scope (see below) at ˜P20-P30. Only the animals whose eyes were healthy looking and the retina showing >80% GFP-positive labeling were kept for further optomotor response assays and histology. Optomotor tests were be performed at P30, P50, P55 and P60 to monitor the cone survival and function in the rd10 eyes. These rd10 eyes were harvested ˜P130 for flat-mount histology and counted for remaining cone as described above for rd1.

### Optomotor Responses.

The optomotor responses of mice were measured using the OptoMotry System (CerebralMechanics) with minor modifications, as previously described (Xiong W, et al. (2015) J Clin Invest 125(4):1433-1445; Xue Y, et al. (2015) J Clin Invest 125(2):727-738). Only the photopic vision was tested, at a background light of ˜70 cd/m² in this study. An examiner tested the mouse visual acuity (i.e. maximal spatial frequency) and the contrast sensitivity (i.e. minimal contrast) separately and blindly (i.e. without knowing which AAVs were injected in which eyes) with the aid of a computer program. In the acuity test, the contrast of the grates was set at 100%, and the temporal frequency was set at 1.5 Hz. During the test, a computer program determined the moving direction of the grates (i.e. clockwise or counter-clockwise) and the parameters at each testing episode. The examiner could see the moving direction of the grates through virtual radiances on the screen but could not see the parameters, in order to minimize human bias. In each testing episode (˜5 seconds), the examiner reported “yes” (or “no”) to the system if observation of the mouse provided (or not) an optomotor response that matched the grating movement. After a series of test episodes, the same computer program determined the acuity of the right eyes (i.e. counter-clockwise) and the left eye (i.e. clockwise). The acuity was recorded as it was for analysis.

### Fundus Imaging.

Fundus images of mouse eyes were taken by a commercially available MicronIV fundus imaging system (Phoenix Research Labs). The animals were anesthetized with a ketamine/xylazine (100/10 mg/kg) cocktail. The eyes were treated with a drop of 5% phenylephrine and 0.5% tropicamide solution to dilate the pupils, and a drop of GONAK 2.5% hypromellose solution (Akorn) to keep the lens hydrated. Fundus images were taken with a filter set of Exciter (FF01-469/35-25, Semrock) and Barrier (FF03-525/50, Semrock) that were selected for spectra to visualize GFP. The optical coherence tomography (OCT) image of the retina was taken near the optic nerve head, and the imaging location was marked on the fundus image by a long green arrow.

### Example 1: Identification of Mutation-Independent Genes Useful for Treating Subjects Having Retinits Pigmentosa (RP)

In order to identify mutation-independent genes useful for the treatment of RP, AAV vectors expressing various genes postulated as candidates for trating RP, including numerous glycolytic enzymes, such as Hexokinase-1 (HK1); Hexokinase-2 (HK2); 6-phosphofructokinase, muscle type (PKFM); pyruvate kinase muscle isozyme M2 (PKM2); HK1 and PKFM; PKFM and pyruvate kinase muscle isozyme M1 (PKM1); HK2, PFKM, and PKM1; lactate dehydrogenase A (LDHA); Basigin1 (BSG1); Rod-derived cone viability factor (RdCVF); or thioredoxin-interacting protein (TXNIP) were produced and subretinally administered to rd1 mice along with an AAV expressing GFP for quantification. The Table below summarizes the AAV-promoter-gene expression cassettes used.

AAV8-RedO-mRdCVF/s
AAV8-RedO-mBasigin1
AAV8-SynPVI-hHK1
AAV8-SynPVI-mHK2
AAV8-SynPVI-hPFKM
AAV8-SynPVI-hPKM1
AAV8-SynPVI-mPKM2
AAV8-SynPVI-hNrf2
AAV8-RedO1.7-mLDHA
AAV8-RedO-mLDHB
AAV8-RedO-siLDHB
AAV8-RedO1.7-mGlut1
AAV8-SynPVI-mHIF1A
*“m” is mouse; “h” is human

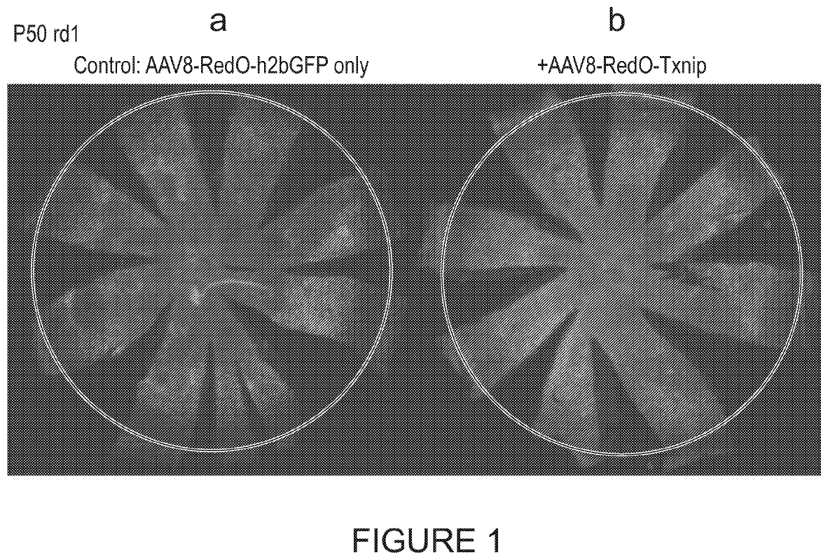

At P50, GFP-positive cones cells within the ½ radius of the retina were counted and, surprisingly, with the exception of thioredoxin-interacting protein (TXNIP), none of the tested AAVs delayed cone degeneration and/or improved cone survival (FIGS. 1 and 2).

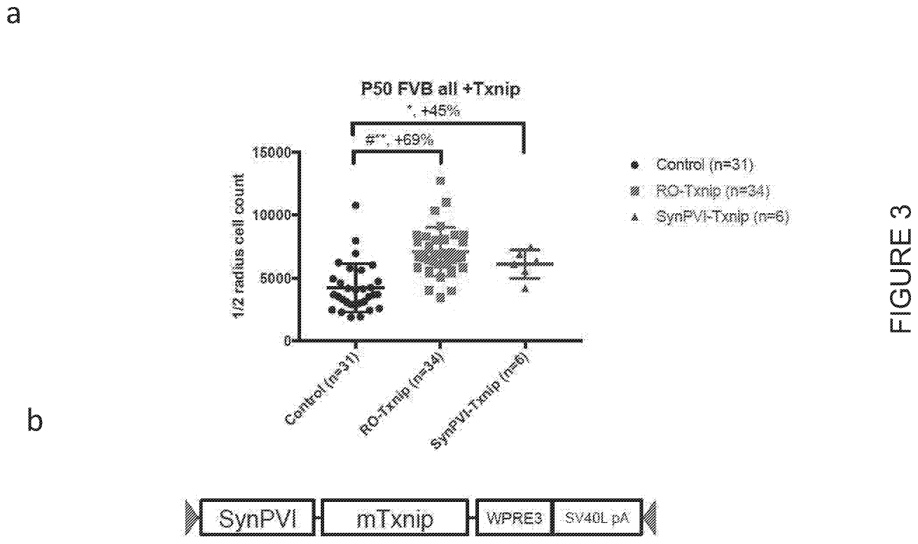

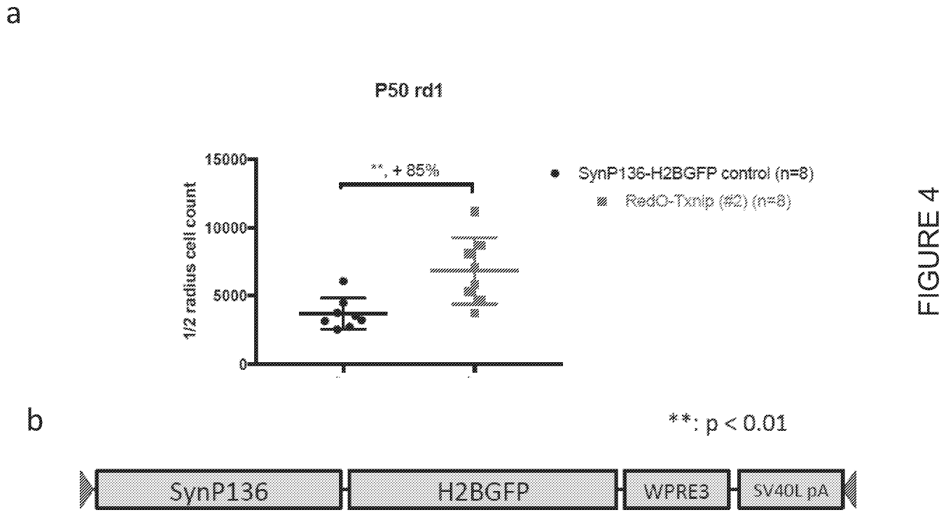

This effect of TXNIP was not limited to use of a RedO promoter as the use of a cone-specific promoter, SynPV1, comprising about 500 bases of the upstream region of the guanine nucleotide-binding protein G subunit alpha-2 (GNAT2) operably linked to TXNIP (FIGS. 4-6), or a cone-specific promoter, SynP136 (see, e.g., Juttner, et al. (2018) https://www.biorxiv.org/content/10.1101/434720v1), comprising about 2 kb of the upstream region of the guanine nucleotide-binding protein G subunit alpha-2 (GNAT2) operably linked to TXNIP also delayed cone degeneration and/or improved cone survival in rd1 mice (FIGS. 3 and 4).

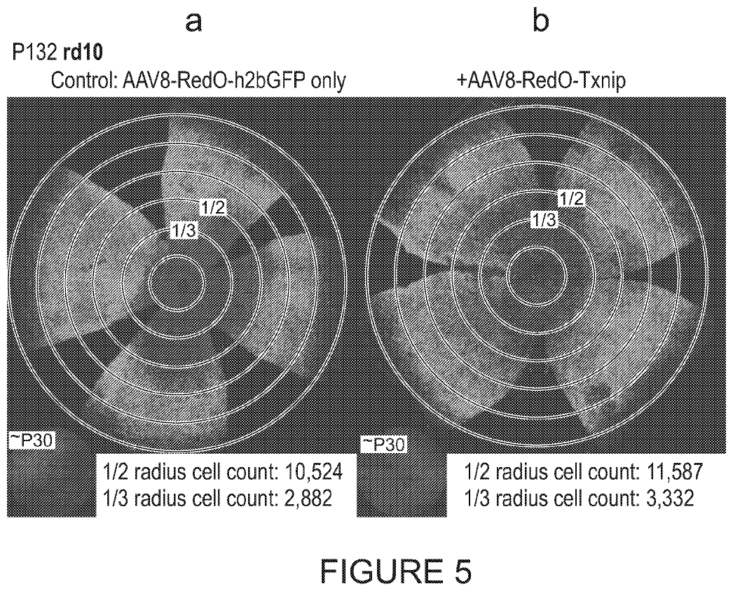

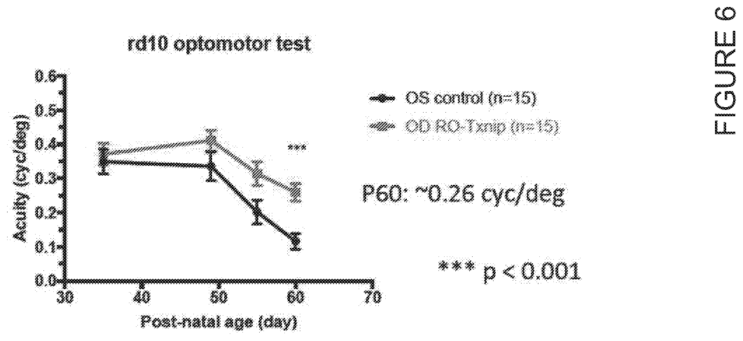

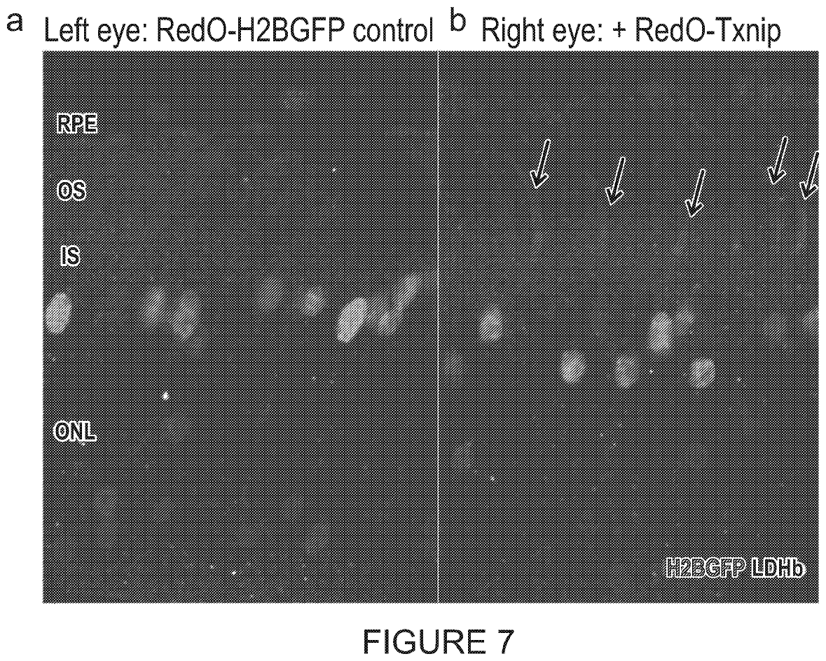

This effect was also not limited to rd1 mice, as subretinal administration of an AAV construct comprising TXNIP (AAV8-RedO_TXNIP) to rd10 mice also significantly delayed cone degeneration and/or improved cone survival (FIG. 5) and, as demonstrated in FIG. 6, preserves functional vision.

### Example 2: Lactate Dehydrogenase B (LDHB) is Necessary for the Rescue of Cone Survival by TXNIP

In order to determine the mechanism of the observed TXNIP rescue, wild-type mice were injected with AAV8-RedO-Txnip and retinas were immunohistochemically stained for various downstream proteins. As depicted in FIG. 7, one protein, lactate dehydrogenase B (LDHB) was significantly upregulated in the cones of mice overexpressing TXNIP.

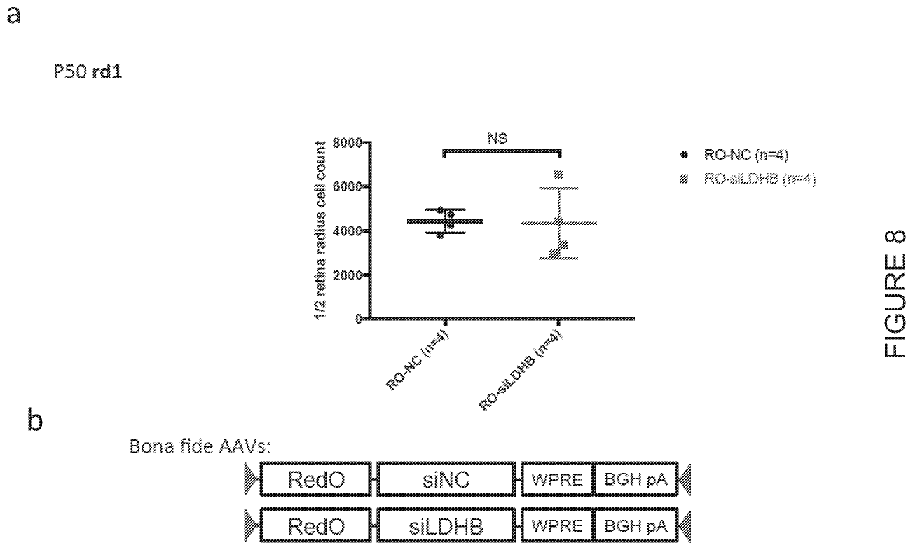

Using an AAV comprising an siRNA targeting LDHB, it was demonstrated that inhibiting the expression of LDHB in rd1 cones alone does not affect cone survival (FIG. 8), but when LDHB was inhibited in rd1 cones overexpressing TXNIP, it was surprisingly discovered that LDHB is necessary for TXNIP rescue of cones (FIG. 8).

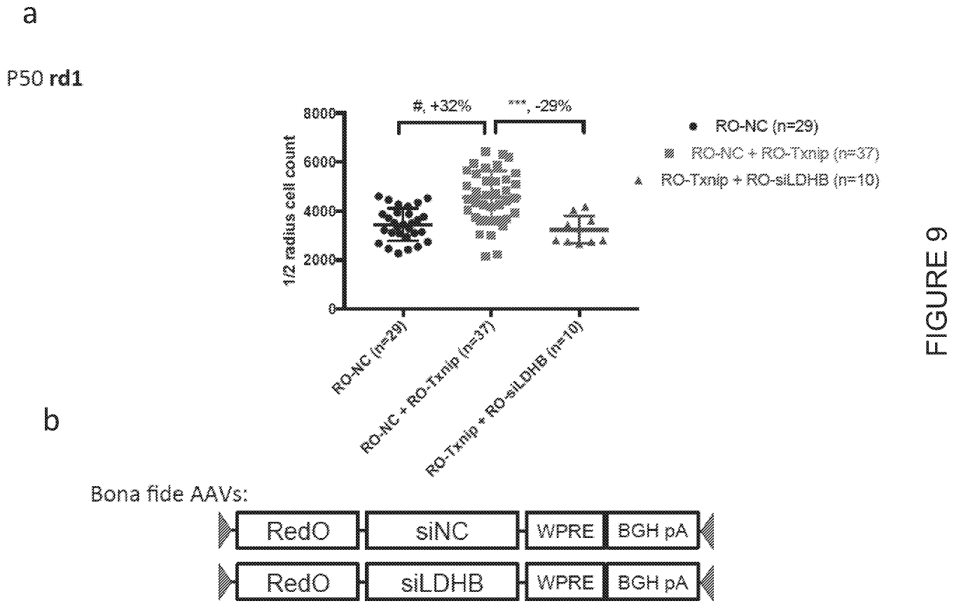

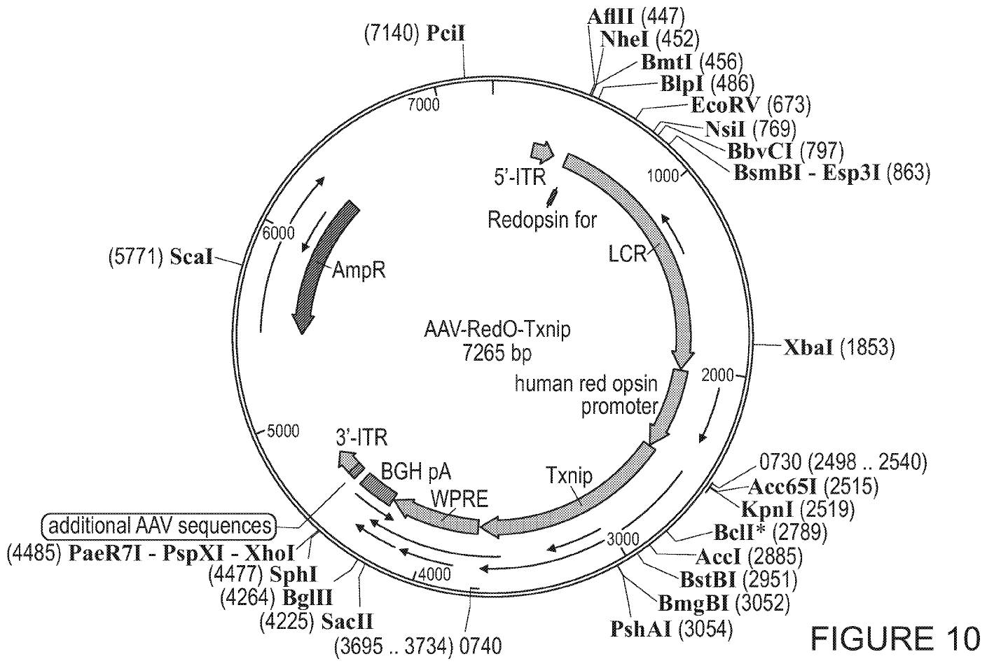

To validate the correlation between LDHB level and TXNIP's recue of cone survival, droplet digital polymerase chain reaction (ddPCR) was performed to test the mRNA levels of LDHB in cone cells from the experimental groups in FIG. 8. As shown in FIG. 9, the ratio of LDHA:LDHB decreased (indicating an increase level of LDHB) when TXNIP is expressed in cone cells. When LDHB expression was silenced by the siRNA, the ratio of LDHA:LDHB increased (indicating a decreased level of LDHB). These data suggest that the expression level of LDHB is up-regulated by the expression of TXNIP in the cone cells.

### List of Sequences

### SEQ ID NO:1

>NM_006472.5 Homo sapiens thioredoxin interacting protein (TXNIP), transcript variant 1, mRNA

### SEQ ID NO:2

>NM_001313972.1 Homo sapiens thioredoxin interacting protein (TXNIP), transcript variant 2, mRNA

### SEQ ID NO:3

>NM_001009935.2 Mus musculus thioredoxin interacting protein (Txnip), transcript variant 1, mRNA

### SEQ ID NO:4

>NM_023719.2 Mus musculus thioredoxin interacting protein (Txnip), transcript variant 2, mRNA

### SEQ ID NO:5

>NM_002300.7 Homo sapiens lactate dehydrogenase B (LDHB), transcript variant 1, mRNA

### SEQ ID NO:6

>NM_001174097.2 Homo sapiens lactate dehydrogenase B (LDHB), transcript variant 2, mRNA

### SEQ ID NO:7

>NM_008492.3 Mus musculus lactate dehydrogenase B (Ldhb), transcript variant 1, mRNA

### SEQ ID NO::8

>NG_009105.2 Homo sapiens opsin 1, long wave sensitive (OPN1LW), RefSeqGene on chromosome X

### SEQ ID NO:9

>NC_000001.11: c109619929-109602906 Homo sapiens chromosome 1, GRCh38.p12 Primary Assembly

### SEQ ID NO:10

### WP RE

### SEQ ID NO:11

### WP RE

### SEQ ID NO:12

Human beta-globin intron

### SEQ ID NO:13

SV40 poly-adenylation (polyA)

### SEQ ID NO:14

### 5′ ITR

### SEQ ID NO:15

### 3′ ITR

### SEQ ID NO:16

>KT886395.1 Homo sapiens clone PR1.7 red cone opsin gene, promoter region and partial cds

### SEQ ID NO:17

>hg38_knownGene_ENST00000351050.7 range=chr1:109613058-109615057 5′pad=0 3′pad=0 strand=−repeatMasking=none

### SEQ ID NO:18

### SynPVI:

### SEQ ID NO:19

### SynP136:

### SEQ ID NO:20

### SV40 Intron

### SEQ ID NO:21

### 2A

### SEQ ID NO:22

### P2A

### SEQ ID NO:23

### T2a

### SEQ ID NO:24

### E2a

### SEQ ID NO:25

### Bovine Growth Hormone Polyadenylation Signal (BGH pA)

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A composition, comprising an adeno-associated virus (AAV) expression cassette, the expression cassette comprising a photoreceptor-specific (PR-specific) promoter and a nucleic acid molecule encoding thioredoxin-interacting protein (TXNIP).

2. A composition, comprising an adeno-associated virus (AAV) expression cassette, the expression cassette comprising a photoreceptor-specific (PR-specific) promoter and a nucleic acid molecule encoding lactate dehydrogenase B (LDHB).

3. (canceled)

4. (canceled)

5. The composition of claim 1 or 2, wherein the PR-specific promoter is a human red opsin (hRedO) promoter,
wherein the hRedO promoter comprises nucleotides 452-2017 of SEQ ID NO:8 directly linked to nucleotides 4541-5032 of SEQ ID NO:12; or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 452-2017 of SEQ ID NO:8 directly linked to nucleotides 4541-5032 of SEQ ID NO:8; or
wherein the hRedO promoter comprises the nucleotide sequence of SEQ ID NO:16, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of SEQ ID NO:16; or
wherein the hRedO promoter comprises nucleotides 457-2514 of the nucleotide sequence depicted in FIG. 11 (SEQ ID NO:26), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 457-2514 of the nucleotide sequence depicted in FIG. 11 (SEQ ID NO:26); or
wherein the hRedO promoter comprises nucleotides 457-2514 of the nucleotide sequence depicted in FIG. 15 (SEQ ID NO: 49), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 457-2514 of the nucleotide sequence depicted in FIG. 15 (SEQ ID NO: 49).

6.-9. (canceled)

10. The composition of claim 1 or 2, wherein the PR-specific promoter is a human guanine nucleotide-binding protein G subunit alpha-2 (GNAT2) promoter,
wherein the GNAT 2 promoter comprises nucleotides 4873-6872 of SEQ ID NO:9; or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 4873-6872 of SEQ ID NO:9; or
wherein the GNAT 2 promoter comprises the nucleotide sequence of SEQ ID NO:17; or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of the nucleotide sequence of SEQ ID NO:17; or
wherein the GNAT 2 promoter comprises the nucleotide sequence of SEQ ID NO:18; or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of the nucleotide sequence of SEQ ID NO:18; or
wherein the GNAT 2 promoter comprises the nucleotide sequence of SEQ ID NO:19; or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of the nucleotide sequence of SEQ ID NO:19; or
wherein the GNAT 2 promoter comprises nucleotides 156-655 of the nucleotide sequence depicted in FIG. 13 (SEQ ID NO: 39), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 156-655 of the nucleotide sequence depicted in FIG. 13 (SEQ ID NO: 39).

11.-15. (canceled)

16. The composition of claim 1, wherein the nucleic acid molecule encoding TXNIP comprises nucleotides 366-1541 of SEQ ID NO:1; or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 366-1541 of SEQ ID NO:1 or
wherein the nucleic acid molecule encoding TXNIP comprises nucleotides 162-1172 of SEQ ID NO:2, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 162-1172 of SEQ ID NO:2; or
wherein the nucleic acid molecule encoding TXNIP comprises nucleotides 280-1473 of SEQ ID NO:3; or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 280-1473 of SEQ ID NO:3; or
wherein the nucleic acid molecule encoding TXNIP comprises nucleotides 280-1470 of SEQ ID NO:4, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 280-1470 of SEQ ID NO:4; or
wherein the nucleic acid molecule encoding TXNIP comprises nucleotides 2521-3714 of the nucleotide sequence depicted in FIG. 11 (SEQ ID NO:26), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 2521-3714 of the nucleotide sequence depicted in FIG. 11 (SEQ ID NO:26); or
wherein the nucleic acid molecule encoding TXNIP comprises nucleotides 663-1856 of the nucleotide sequence depicted in FIG. 13 (SEQ ID NO: 39), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 663-1856 of the nucleotide sequence depicted in FIG. 13 (SEQ ID NO: 39).

17.-21. (canceled)

22. The composition of claim 2, wherein the nucleic acid molecule encoding LDHB comprises nucleotides 112-1116 of SEQ ID NO:5; or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 112-1116 of SEQ ID NO:5; or
wherein the nucleic acid molecule encoding LDHB comprises nucleotides 334-1338 of SEQ ID NO:6, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 334-1338 of SEQ ID NO:6; or
wherein the nucleic acid molecule encoding LDHB comprises nucleotides 112-1116 of SEQ ID NO:7, or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 112-1116 of SEQ ID NO:7; or
wherein the nucleic acid molecule encoding LDHB comprises nucleotides 2517-3521 of the nucleotide sequence depicted in FIG. 15 (SEQ ID NO: 49), or a nucleotide sequence having about 85%, 86%, 87%, 88%, 89%, 90%, 91%, 92%, 93%, 94%, 95%, 96%, 97%, 98%, or about 99% nucleotide sequence identity to the entire nucleotide sequence of nucleotides 2517-3521 of the nucleotide sequence depicted in FIG. 15 (SEQ ID NO: 49).

23.-25. (canceled)

26. The composition of claim 1 or 2, wherein the expression cassette further comprises a linker, an intron, a post-transcriptional regulatory region, or a polyadenylation signal; or combinations thereof.

27.-32. (canceled)

33. The composition of claim 1 or 2, wherein the expression cassette is present in an AAV vector.

34. (canceled)

35. An AAV vector particle comprising the composition of claim 1 or 2.

36. An isolated cell comprising the AAV particle of claim 35.

37. A pharmaceutical composition formulated for intraocular administration comprising the AAV composition of claim 1 or 2.

38.-41. (canceled)

42. A method for treating or preventing a degenerative ocular disorder in a subject, comprising administering to said subject a therapeutically effective amount of claim 1 or 2, thereby treating or preventing said degenerative ocular disorder.

43.-49. (canceled)
